(19) United States
(12) Patent Application Publication
(10) Pub. No.: US 2022/0088265 A1
(43) Pub. Date: Mar. 24, 2022

(54) BIOMATERIALS AND METHODS RELATED THERETO

(71) Applicants: Manmeet KAUR, Auckland (NZ); Trevor SHERWIN, Auckland (NZ); Judith Louise GLASSON, Auckland (NZ); Kishara SAMARASEKARA, Auckland (NZ); Laura Joy DOMIGAN, Auckland (NZ); AUCKLAND UNISERVICES LIMITED, Auckland (NZ)

(72) Inventors: Manmeet KAUR, Auckland (NZ); Laura Joy DOMIGAN, Auckland (NZ); Trevor SHERWIN, Auckland (NZ); Judith Louise GLASSON, Auckland (NZ); Kishara SAMARASEKARA, Auckland (NZ)

(21) Appl. No.: 17/429,007

(22) PCT Filed: Feb. 5, 2020
(86) PCT No.: PCT/NZ2020/050005

§ 371 Date: Aug. 6, 2021

(30) Foreign Application Priority Data
Feb. 8, 2019 (NZ) 750438
May 27, 2019 (NZ) 753898

(51) Int. Cl. A61L 24/10; A61K 47/42; C12N 5/079; C12N 5/0775; A61K 35/28; A61L 27/22; A61L 27/38

(52) U.S. Cl. CPC A61L 24/108; A61K 47/42; C12N 5/0621; C12N 5/0668; A61K 35/28; A61L 2300/406; A61L 27/3834; A61L 2300/252; C12N 2533/50; C12N 2513/00; A61L 27/227

(57) ABSTRACT

The present invention relates to biocompatible compositions comprising one or more crystallin proteins, and the use of such compositions in therapeutic and research methods, for example in surgical methods, in sustained release drug delivery, and in cell-based methods.

### TECHNICAL FIELD

The invention relates to proteinaceous biomaterials and methods for their preparation and use. More particularly, the present invention relates to biomaterials comprising crystallin proteins, methods for preparing said biomaterials, and various methods of using said biomaterials in a range of disciplines, including in medicine and therapeutic methods, such as surgical methods, cell-based therapies, and drug delivery, and in biomedical research, in for example cell- and tissue culture methods, and in tissue engineering.

### BACKGROUND OF THE INVENTION

The following includes information that may be useful in understanding the present inventions. It is not an admission that any of the information provided herein is prior art, or relevant, to the presently described or claimed inventions, or that any publication or document that is specifically or implicitly referenced is prior art. Any discussion of the prior art throughout the specification should in no way be considered as an admission that such prior art is widely known or forms part of the common general knowledge in the field.

Biocompatible materials are critical to many therapeutic and scientific methods. For example, sutures able to safely dissolve over a defined period are essential to many surgical methods, while implants and implantable compositions, such as implantable compositions for sustained drug delivery, are advantageously biocompatible, non-inflammatory, and safe. Further, when such implants and implantable compositions are designed to degrade over or after their functional lifetime, ideally the degredation products themselves are likewise biocompatible and safe.

Biocompatibility is important irrespective of whether the material is to be used directly, for example, on or in a subject, or indirectly, such as in the preparation of a cell-based therapy or of engineered tissue. In the former case, for example, biocompatible adhesives can reduce complications associated with irritation, scarring and discomfort. In the latter case, for example, scaffolds for tissue and organ engineering are commonly synthesised from biodegradable synthetic polymers, but many such synthetic polymers have low biocompatibility and/or low mechanical strength, or less than optimal degredation profiles under physiological conditions, thus limiting their use.

Despite the clear recognition that biocompatibility of materials used in medical therapies and in a number of research methods is desirable, for many such therapies and methods biocompatible options either do not exist or suffer from one or more deficiencies. For example, in the context of ocular surgery, existing surgical adhesives are typically synthetic, and have been associated with scarring and toxicity.

There is thus a need to develop new biocompatible materials for a number of therapeutic and research uses, including, for example, surgical and drug delivery uses.

It is therefore an object of the invention to provide one or more biocompatible materials comprising crystallin protein, or to at least provide a useful alternative to existing biomaterials and methods reliant thereon, or to at least provide the public with a useful choice.

### SUMMARY OF THE INVENTION

In a first aspect the invention relates to a biocompatible composition comprising: one or more isolated, purified, recombinant, or synthesised proteins selected from the group comprising:
a) an α-crystallin;
b) a β-crystallin;
c) a γ-crystallin;
d) a protein from any one of a) to c) above from Hoki (Macruronus novaezelandiae);
e) a protein from any one of a) to c) above from Homo sapiens;
f) a protein comprising the amino acid sequence identified in Table 1 herein;
g) a polypeptide comprising or consisting of at least about 10 contiguous amino acids from any one of a) to f) above;
h) a protein having at least about 90% amino acid identity to any one of a) to g) above;
i) a protein according to any one of a) to h) above having the native structure of a crystallin protein in vivo;
j) any combination of two or more of a) to i) above;

optionally one or more plasticizers;

optionally one or more co-initiators; and

one or more crosslinkers.

In another aspect the invention relates to a biopolymer composition comprising a protein selected from the group comprising:
a) an α-crystallin;
b) a β-crystallin;
c) a γ-crystallin;
d) a protein from any one of a) to c) above from Hoki (Macruronus novaezelandiae);
e) a protein from any one of a) to c) above from Homo sapiens;
f) a protein comprising the amino acid sequence identified in Table 1 herein;
g) a polypeptide comprising or consisting of at least about 10 contiguous amino acids from any one of a) to f) above;
h) a protein having at least about 90% amino acid identity to any one of a) to g) above;
i) a protein according to any one of a) to h) above having the native structure of a crystallin protein in vivo;
j) any combination of two or more of a) to i) above;

optionally one or more plasticizers;

optionally one or more co-initiators; and one or more crosslinkers;

wherein said one or more proteins are crosslinkable to form a polymer.

In various embodiments, the one or more proteins are crosslinkable in vivo.

In one aspect, the invention relates to an in vivo gelling composition, the composition comprising:
a) an α-crystallin;
b) a β-crystallin;
c) a γ-crystallin;
d) a protein from any one of a) to c) above from Hoki (Macruronus novaezelandiae);
e) a protein from any one of a) to c) above from Homo sapiens;
f) a protein comprising the amino acid sequence identified in Table 1 herein;
g) a polypeptide comprising or consisting of at least about 10 contiguous amino acids from any one of a) to f) above;
h) a protein having at least about 90% amino acid identity to any one of a) to g) above;
i) a protein according to any one of a) to h) above having the native structure of a crystallin protein in vivo;
j) any combination of two or more of a) to i) above;

optionally one or more plasticizers;

optionally one or more co-initiators; and

one or more crosslinking molecules;

wherein the in vivo gelling composition at least in part polymerises and/or gels at a target site in or on a subject's body, or wherein crosslinking of the in vivo gelling composition occurs or is initiated when present at a target site in or on a subject's body.

Accordingly, in one embodiment, the biocompatible composition is an in vivo gelling composition formulated to at least in part polymerise and/or gel at a target site in or on a subject's body, or wherein the biocompatible composition is an in vivo gelling composition formulated such that crosslinking of the in vivo gelling composition occurs or is initiated when present at a target site in or on a subject's body

Another aspect of the present invention relates to a method for producing a crosslinked biopolymer composition, the method comprising:

providing a composition comprising:
a) an α-crystallin;
b) a β-crystallin;
c) a γ-crystallin;
d) a protein from any one of a) to c) above from Hoki (Macruronus novaezelandiae);
e) a protein from any one of a) to c) above from Homo sapiens;
f) a protein comprising the amino acid sequence identified in Table 1 herein;
g) a polypeptide comprising or consisting of at least about 10 contiguous amino acids from any one of a) to f) above;
h) a protein having at least about 90% amino acid identity to any one of a) to g) above;
i) a protein according to any one of a) to h) above having the native structure of a crystallin protein in vivo;
j) any combination of two or more of a) to i) above;
k) optionally one or more plasticizers;
l) optionally one or more co-initiators; and contacting said composition with one or more crosslinking molecules;
initiating crosslinking, thereby forming a crosslinked biopolymer composition.

A further aspect of the present invention relates to a method for producing a composition comprising one or more purified crystallin proteins, the method comprising:
i. providing vertebrate eye tissue;
ii. homogenising the tissue in the presence of an extraction buffer under conditions suitable for the maintenance of native crystallin protein structure;
iii. separating the liquid homogenate from any residual solids, for example by centrifugation or filtration, to provide a crystallin protein-containing solution;
iv. optionally at least partially further purifying the crystallin protein;
v. optionally dialysing the crystallin protein-containing solution to remove the extraction buffer;
vi. optionally lyophilising the crystallin protein-containing solution to provide a lyophilised crystallin protein composition;
vii. optionally storing the crystallin protein-containing solution or the lyophilised crystallin protein composition, for example at or below 0° C.;

wherein a substantial proportion of the purified crystallin proteins retain their native structure.

Accordingly, in one embodiment the method for producing a composition comprising one or more purified crystallin proteins comprises:
i. providing vertebrate eye tissue;
ii. homogenising the tissue in the presence of an extraction buffer having a physiological pH, wherein the homogenate is maintained at a temperature of below about 15° C.;
iii. separating the liquid homogenate from any residual solids, for example by centrifugation or filtration, to provide a crystallin protein-containing solution;
iv. optionally at least partially further purifying the crystallin protein;
v. optionally dialysing the crystallin protein-containing solution to remove the extraction buffer;
vi. optionally lyophilising the crystallin protein-containing solution to provide a lyophilised crystallin protein composition;
vii. optionally storing the crystallin protein-containing solution or the lyophilised crystallin protein composition, for example at or below 0° C.;

wherein a substantial proportion of the purified crystallin proteins retain their native structure.

In one embodiment, the conditions suitable for the maintenance of native crystallin protein structure comprise
a. maintaining the homogenate in an extraction buffer at a pH of 7 or greater; or
b. maintaining the homogenate at a physiological pH; or
c. maintaining the homogenate at a temperature of below about 15° C.;
d. both a) and c) above; or
e. both b) and c) above.

In one embodiment, the conditions suitable for the maintenance of native crystallin protein structure comprise
a. maintaining the homogenate in an extraction buffer at a pH of 7 or greater; or
b. maintaining the homogenate at a physiological pH; or
c. maintaining the homogenate at a temperature of below about 15° C.;
d. both a) and c) above; or
e. both b) and c) above;

and the method comprises:

separating the liquid homogenate from any residual solids by centrifugation or filtration, to provide a crystallin protein-containing solution;

dialysing the crystallin protein-containing solution to remove the extraction buffer; optionally lyophilising the crystallin protein-containing solution to provide a lyophilised crystallin protein composition;

maintaining the crystallin protein-containing solution or the lyophilised crystallin protein composition under conditions appropriate to maintenance of native crystallin protein structure, for example at or below about 4° C. until use;

wherein a substantial proportion of the purified crystallin proteins retain their native structure.

Accordingly, in one embodiment, the method for producing a composition comprising one or more purified crystallin proteins comprises:
i. providing vertebrate eye tissue;
ii. homogenising the tissue in the presence of an extraction buffer at a pH of 7 or greater, wherein the homogenate is maintained at a temperature of below about 15° C.;
iii. separating the liquid homogenate from any residual solids by centrifugation or filtration, to provide a crystallin protein-containing solution;
iv. dialysing the crystallin protein-containing solution to remove the extraction buffer;
v. optionally lyophilising the crystallin protein-containing solution to provide a lyophilised crystallin protein composition;
vi. maintaining the crystallin protein-containing solution or the lyophilised crystallin protein composition at or below about 4° C. until use;

wherein a substantial proportion of the purified crystallin proteins retain their native structure.

In one embodiment, the vertebrate eye tissue is lens tissue.

In one embodiment, the vertebrate eye tissue is phacoemulsification material.

In one embodiment, the vertebrate eye tissue is eye tissue from fish, for example, deep sea fish. In one embodiment, the vertebrate eye tissue is eye tissue from Hoki, such as Hoki eye lens tissue.

In one embodiment, the vertebrate eye tissue is eye tissue from a mammal, such as human, bovine, porcine, caprine, ovine, or cervine eye tissue.

In one embodiment, the vertebrate eye tissue is eye tissue from Homo sapiens, such as Homo sapiens eye lens tissue.

In one embodiment, the vertebrate eye tissue is from phacoemulsification surgery.

In one embodiment, the extraction buffer comprises one or more of Tris(hydroxymethyl)aminomethane, NaCl, sodium azide, or any combination of two of more thereof.

In one embodiment, the extraction buffer is at physiological pH, such as the physiological pH of the organism from which the crystallin protein is obtained. In one embodiment, the pH of the extraction buffer is greater than 7. For example, the pH of the extraction buffer is in the range from about 7 to about 9.

In one embodiment, the extraction buffer is present at a ratio of at least about 2 mL buffer per gram of eye tissue.

In one embodiment, homogenisation is performed under conditions to avoid or minimise disruption of one or more crystallin isoforms and/or to avoid or minimise protein aggregation. For example, disruption and/or aggregation are avoided, to the extent possible, in order to avoid or minimise formation of crystallin nanofibrils or to avoid or minimise the formation of non-naturally occurring protein conformers.

In one example, homogenisation is performed at physiological pH, for example, in a buffer having a physiological pH. In one example, homogenisation is performed at a pH of greater than about 7. In one example, homogenisation is performed under low shear conditions. In one example, homogenisation is performed in the presence of one or more stabilising additives, such as, for example, arginine.

In one example, homogenisation is performed at low temperature. In one embodiment, homogenisation occurs at a temperature of from about 0° C. to about 5° C. In one embodiment, the homogenisation is interspersed with rest phases in which no homogenising is performed, for example, interspersed with a chilling phase where the homogenate is placed on ice for a period.

In one embodiment, dialysis is performed against water, for example, against purified water, such as for example, Milli-Q water, at less than 5° C.

In one embodiment, the purified crystallin protein is stored in the presence of a stabiliser. For example, the purified crystallin protein is stored in the presence of arginine, glycine, or a combination thereof.

In various embodiments, the substantial proportion of the purified crystallin proteins that retain their native structure is greater than about 60%, greater than about 70%, greater than about 80%, greater than about 90%, greater than about 95%, or greater than about 99%. In various embodiments, retention of native structure is determined by a method well known in the art, including but not limited to circular dichroism, NMR, or native PAGE.

In one embodiment, the method for producing a composition comprising one or more purified crystallin proteins is essentially as herein described in the Examples.

In a further aspect, the invention relates to a method of tissue closure in a subject in need thereof, the method comprising

optionally applying force to close the laceration, lesion, incision or wound;

contacting a laceration, lesion, incision, or wound or the site of said laceration, lesion, incision, or wound with a crystallin protein containing composition as herein described, optionally wherein the crystallin containing composition is at least partially crosslinked,

optionally applying force to close the laceration, lesion, incision or wound,

initiating and/or maintaining crosslinking;

maintaining the closure of the laceration, lesion, incision or wound for a time sufficient for crosslinking to occur;

wherein application and/or crosslinking of the crystallin proteins forms an adhesive composition.

In a further aspect, the invention relates to a method of tissue closure in a subject in need thereof, the method comprising

optionally applying force to close the laceration, lesion, incision or wound;

contacting a laceration, lesion, incision, or wound or the site of said laceration, lesion, incision, or wound with a crystallin protein containing composition as herein described,

optionally applying force to close the laceration, lesion, incision or wound,

initiating crosslinking;

maintaining the closure of the laceration, lesion, incision or wound for a time sufficient for crosslinking to occur;

wherein crosslinking of the crystallin proteins forms an adhesive composition.

In one embodiment, the method of tissue closure is a method of closing a surgical incision.

In one embodiment, the method of tissue closure is a method of sutureless closure. For example, the sutureless closure is sutureless skin closure, sutureless wound closure, or sutureless operative incision closure.

In one embodiment, the surgery is ophthalmic surgery. In one example, the ophthalmic surgery is cataract surgery, conjunctival grafts, vitrectomy including pars planar vitrectomy, refractive lens exchange, lens implantation, or lens replacement surgery. In another example, the ophthalmic surgery is retinal detachment surgery, including retinal surgery incorporating retinopexy or scleral buckling, macular hole surgery, conjunctival closures, glaucoma surgery, bleb leak surgery, trabeculectomy, blepharorrhaphy, amniotic membrane transplantation, corneal perforation surgery, pterygium surgery including pterygium excision, posterior capsule intraocular lens implantation, epithelial ingrowth surgery, keratoplasty including lamellar keratoplasty, deep anterior lamellar keratoplasty, strabismus surgery including bilateral strabismus surgery, eyelid skin graft surgery, or mucous membrane graft surgery.

In one embodiment, the composition is applied via an ophthalmic surgical device, such as an anterior chamber cannula (Rycroft cannula).

In various embodiments, maintenance of the closure of the laceration, lesion, incision or wound is for a time sufficient for crosslinking to occur is by the application of one or more medical aids, such as bandages, sutures, meshes or the like, or by (usually temporary) physical force, such as clamping or holding the laceration, lesion, incision or wound closed.

In various embodiments, maintenance of the closure of the laceration, lesion, incision or wound is for a time sufficient for greater than about 60% crosslinking to occur, for example, greater than about 70% crosslinking to occur, greater than about 80% crosslinking to occur, greater than about 90% crosslinking to occur, or greater than about 95% crosslinking to occur.

In one embodiment, the crosslinker present in the composition is a photocrosslinker, wherein initiation of crosslinking is by exposure to light. For example, in one embodiment the crosslinker is a UV crosslinker, such as but not limited to 1-hydroxycyclohexyl-1-phenyl ketone (e.g., Irgacure 184), 2, 2 dimethoxy-2-phenylacetophenone (e.g., Irgacure 651), riboflavin, or lithium phenyl-2,4,6-trimethylbenzoylphosphinate (LAP), and initiation is by exposure to UV light (365 nm). In another embodiment, the crosslinker is a visible light crosslinker, such as a visible light activated system comprising eosin Y (EY) and triethanolamine (TEOA), and initiation is by exposure to visible light (530 nm).

In one embodiment, the invention relates to a method of tissue closure in a subject in need thereof, wherein the subject is undergoing or who has undergone ophthalmic surgery, the method comprising

contacting a surgical incision or the site of said surgical incision with a crystallin protein containing composition as herein described, optionally wherein the crystallin containing composition is at least partially crosslinked;

optionally applying force to close the incision;

initiating and/or maintaining crosslinking;

maintaining the closure of the surgical incision for a time sufficient for crosslinking to occur;

wherein application and/or crosslinking of the crystallin proteins forms an adhesive composition capable of maintaining closure of the surgical incision.

In another embodiment, the invention relates to a method of tissue closure in a subject in need thereof, wherein the subject is undergoing or who has undergone ophthalmic surgery, the method comprising

contacting a surgical incision or the site of said surgical incision with a crystallin protein containing composition as herein described;

optionally applying force to close the incision;

initiating crosslinking;

maintaining the closure of the surgical incision for a time sufficient for crosslinking to occur;

wherein crosslinking of the crystallin proteins forms an adhesive composition capable of maintaining closure of the surgical incision.

In one embodiment, the adhesive composition is capable of maintaining closure of the surgical incision in the absence of sutures or other closure aids.

In one embodiment, the adhesive composition has a refractive index equivalent to that of the subject's eye. In one embodiment, the adhesive composition has high transmissivity across the visible spectrum.

In a further aspect, the invention relates to a method of treating an ocular injury or ocular incision in a subject in need thereof, the method comprising the steps:
i. contacting the ocular injury or incision with a crystallin containing composition as herein described, optionally wherein the crystallin containing composition is at least partially crosslinked;
   and
ii. initiating and/or maintaining crosslinking;

wherein crosslinking forms a bioadhesive polymer composition.

In another aspect, the invention relates to a method of treating an ocular injury or ocular incision in a subject in need thereof, the method comprising the steps:
i. contacting the ocular injury or incision with a composition as described herein; and
ii. initiating crosslinking;

wherein the crosslinking forms a bioadhesive polymer composition.

In another aspect, the invention relates to a method of delivering one or more active agents to a subject in need thereof, the method comprising

providing a crystallin protein comprising composition as herein described, wherein the composition additionally comprises one or more active agents,

contacting the subject with the composition,

optionally initiating crosslinking of the composition,

thereby delivering the active agent to the subject in need thereof.

In certain embodiments, contacting the subject with the composition comprises administering the composition to a target site on or in the subject's body, including, for example, surgical administration.

In a still further aspect the invention relates to a method of culturing one or more cells or tissues, the method comprising

providing one or more cells to be cultured;

contacting the one or more cells with a substrate comprising a composition as herein described;

maintaining the one or more cells in contact with the substrate and optionally in contact with additional growth media for a period under conditions suitable for continued viability, growth, replication, and/or differentiation. In various embodiments, the composition as described herein comprises γ-crystallin. In one embodiment, γ-crystallin comprises at least about 10% w/w of the crystallin protein present in the composition.

In one embodiment, the one or more cells comprise one or more replicatively-competent cells.

In one embodiment, the one or more cells are one or more stem cells.

In one embodiment, the substrate is a thin film formed from a composition as described herein, for example a thin film of sufficient mechanical strength and/or elasticity to enable the transfer of cells in contact therewith to another location. In one example, the location is a second culture vessel. In another example, the location is on or in a subject's body, such as, for example, a surgical site. For example, the one or more cells are one or more ophthalmic cells or one or more stem cells derived from the eye, and the surgical site is in or on the eye. In one example, the one or more cells are one or more limbal stem cells. In another example, the one or more cells are one or more stromal stem cells. In still another example, the one or more cells are one or more retinal pigment epithelial cells. In another example, the one or more cells are one or more totipotent stem cells, one or more pluripotent stem cells, or one or more multipotent stem cells.

In one embodiment, the substrate is a gel formed from a composition as described herein. For example, the substrate is a gel having at least one region of sufficient thickness to allow for the formation of a 3D cell culture.

Accordingly, in one embodiment the method of culturing one or more cells or tissues is a method of culturing one or more cells from the vertebrate eye, the method comprising

providing one or more vertebrate eye cells to be cultured;

contacting the one or more cells with a substrate comprising a composition as herein described, wherein the substrate is optically transparent;

maintaining the one or more cells in contact with the substrate and optionally in contact with additional growth media for a period under conditions suitable for continued viability, growth, replication, and/or differentiation;

wherein the substrate is of sufficient mechanical durability to support transfer to the eye of a subject and/or handling associated with surgical application.

In still a further aspect, the invention relates to the use of a composition as herein described in the preparation of a medicament for use in therapy, for example, for use in any one of the therapeutic methods described herein. In one embodiment, the medicament is for use in surgery, such as ophthalmic surgery including any one of the surgical methods herein recited. Also contemplated is the use of a composition as herein described in the preparation of a medicament or composition for in vitro use, including a therapeutic or research method that employs an in vitro step.

In a further aspect, the invention relates to a composition as herein described for use in therapy, for example, for use in any one of the therapeutic methods described herein. In one embodiment, the use is in surgery, such as in ophthalmic surgery including any one of the surgical methods herein recited. Also contemplated is a composition as herein described for use in an in vitro therapeutic or research method, or a therapeutic or research method that employs an in vitro step.

In a further aspect, the invention relates to a method of treating an ocular disorder associated with deficiency of stem cells in a subject in need thereof, the method comprising contacting the eye with a therapeutic composition comprising:
i. a stem cell, optionally cultured according to a method of culturing as described herein;
   and optionally
ii. a biocompatible composition or biopolymer composition as described herein.

In one embodiment, the ocular disorder comprises limbal stem cell deficiency (LSCD) or an associated disorder. In another embodiment, the ocular disorder comprises macular degeneration, such as age-related macular degeneration (ARMD) or a related disorder. In still another embodiment, the ocular disorder comprises inherited retinal disease (IRD) or a related disorder.

In a further embodiment, the therapeutic composition comprises at least one limbal stem cell. In another embodiment, the therapeutic composition comprises at least one stromal stem cell. In another embodiment, the therapeutic composition comprises at least one retinal pigment epithelial cell. In another embodiment, the therapeutic composition comprises one or more of at least one totipotent stem cell, at least one pluripotent stem cell, or at least one multipotent stem cell.

In a further embodiment, the therapeutic composition comprises a thin film formed from a composition as described herein.

In one embodiment, one or more stem cells are cultured in the presence of a biocompatible composition or biopolymer composition as described herein for a period sufficient to allow adherence of the one or more cells to the composition.

For example, the one or more stem cells are cultured in the presence of a biocompatible composition or biopolymer composition as described herein for at least about 7 days, at least about 10 days, at least about 14 days, at least about 21 days, or at least about 28 days.

In one embodiment, one or more stem cells are cultured in the presence of a biocompatible composition or biopolymer composition as described herein for a period sufficient to allow outgrowth of one or more of the cells on or in the composition. For example, the cells are cultured under conditions suitable to allow an increase in cell number.

In particularly contemplated embodiments, the cells are cultured under conditions suitable for and for a period sufficient to allow one or more of an increase in cell number, cellular outgrowth, adherence, an increase in cell density, and/or maintenance of a totipotent, pluripotent, or multipotent stem cell phenotype.

In one embodiment, contacting the eye with the therapeutic composition comprises surgical transplantation to the ocular surface.

In one embodiment, the eye is contacted with the therapeutic composition for a period sufficient to allow transference of one or more of the stem cells to the eye. For example, contact is maintained for a period of at least 24 hours, for example, 48 hours, 72 hours, or 96 hours. In other examples, contact is maintained for 5 days or more, for example, 6 days, 7 days, 8 days, 9 days, 10 days, or more than 10 days. Generally, where possible, contact is maintained for 7 days or more.

In various embodiments, cells are cultured in the presence of biocompatible composition or biopolymer composition as described herein in an amount sufficient to provide for about 1×10² to about 1×10⁷ cells per cm² of composition. For example, cells are cultured in an amount sufficient to provide for about 1×10³ to about 1×10⁶ cells per cm² of composition. In another example, cells are cultured in an amount sufficient to provide for about 1×10⁴ to about 1×10⁶ cells per cm², or about 5×10⁴ to about 1×10⁶ cells per cm², or about 5×10⁴ to about 5×10⁵ cells per cm², or about 1×10⁵ to about 5×10⁵ cells per cm².

In one embodiment, the eye is contacted with a therapeutic composition comprising at least about 1×10² cells per cm². For example, the eye is contacted with a therapeutic composition comprising at least about 1×10³ cells per cm² of composition, for example at least about 1×10⁴ cells per cm² of composition, at least about 1×10⁵ cells per cm² of composition, at least about 1×10⁶ cells per cm² of composition, or at least about 1×10⁷ cells per cm² of composition. In another example, the eye is contacted with a therapeutic composition comprising at least about 1×10³ cells per cm² to about 1×10⁶ cells per cm² of composition. In another example, the eye is contacted with a therapeutic composition comprising from at least about 1×10⁴ to about 1×10⁶ cells per cm², or about 5×10⁴ to about 1×10⁶ cells per cm², or about 5×10⁴ to about 5×10⁵ cells per cm², or about 1×10⁵ to about 5×10⁵ cells per cm².

In a further aspect, the invention relates to a method of treating an ocular disorder in a subject in need thereof, the method comprising

providing a biocompatible composition as described herein, wherein the biocompatible composition comprises one or more active agents,

administering the biocompatible composition to the subject to allow transfer of the one or more active agents to the subject.

In a further aspect, the invention relates to a method of treating an ocular disorder in a subject in need thereof, the method comprising

providing a biocompatible composition as described herein, wherein the biocompatible composition comprises one or more stem cells,

administering the biocompatible composition to the subject to allow transfer of one or more of the stem cells to the subject.

In one embodiment, the biocompatible composition is administered to the subject's eye. For example, the biocompatible composition is surgically administered to the subject's eye, including, for example, by one of the surgical methods as described herein.

In one embodiment, the ocular disorder is associated with a deficiency of one or more cells, such as one or more stem cells.

In one embodiment, the ocular disorder comprises limbal stem cell deficiency (LSCD) or an associated disorder. In another embodiment, the ocular disorder comprises macular degeneration, such as age-related macular degeneration (ARMD) or a related disorder. In still another embodiment, the ocular disorder comprises inherited retinal disease (IRD) or a related disorder.

In a further embodiment, the therapeutic composition comprises at least one limbal stem cell. In another embodiment, the therapeutic composition comprises at least one stromal stem cell. In another embodiment, the therapeutic composition comprises at least one retinal pigment epithelial cell. In another embodiment, the therapeutic composition comprises one or more of at least one totipotent stem cell, at least one pluripotent stem cell, or at least one multipotent stem cell.

In a further embodiment, the therapeutic composition comprises a thin film formed from a composition as described herein.

In various embodiments, at administration to the subject the biocompatible composition comprises from about 1×10² to about 1×10⁸ cells. For example, sufficient biocompatible composition is administered to provide from about 1×10² to about 1×10⁸ cells to the subject's eye. In certain embodiments, biocompatible composition is administered to provide from about 1×10² cells per cm² of composition. For example, biocompatible composition is administered to provide from at least about 1×10³ cells per cm² of composition, for example at least about 1×10⁴ cells per cm² of composition, at least about 1×10⁵ cells per cm² of composition, at least about 1×10⁶ cells per cm² of composition, or at least about 1×10⁷ cells per cm² of composition. In another example, biocompatible composition is administered to provide from at least about 1×10³ cells per cm² to about 1×10⁶ cells per cm² of composition. In another example, biocompatible composition is administered to provide from at least about 1×10⁴ to about 1×10⁶ cells per cm², or about 5×10⁴ to about 1×10⁶ cells per cm², or about 5×10⁴ to about 5×10⁵ cells per cm², or about 1×10⁵ to about 5×10¹ cells per cm².

In one embodiment, administration is to the ocular surface. In one embodiment, the biocompatible composition is an adhesive composition as herein described, with sufficient adhesion to the ocular surface to remain adhered to the ocular surface for a period sufficient to allow transference of one or more of the cells. In one example, administration of the composition does not involve the application of any additional adherents or adhesives. For example, administration of the composition does not involve suturing.

Accordingly, in one embodiment, the method of treating an ocular disorder in a subject in need thereof comprises

providing a biocompatible composition as described herein, wherein the biocompatible composition comprises one or more limbal stem cells, one or more stromal stem cells, one or more retinal pigment epithelial cells, or any combination thereof;

administering the biocompatible composition to the subject's eye to allow transfer of one or more of the stem cells to the subject's eye.

In a further embodiment, the method of treating an ocular disorder in a subject in need thereof comprises

providing a biocompatible composition as described herein, wherein the biocompatible composition comprises one or more totipotent stem cells, one or more pluripotent stem cells, one or more multipotent stem cells, or any combination thereof;

administering the biocompatible composition to the subject's eye to allow transfer of one or more of the stem cells to the subject's eye.

Any of the embodiments described herein can relate to any of the aspects presented herein.

In various embodiments, the composition is an adhesive composition, a gel composition, or a thin film composition.

In one example, the adhesive composition is a surgical adhesive.

In one example, the gel composition is or comprises a contact lens, including a contact lens for surgical use and/or drug delivery.

In one example, the gel composition is for cell adhesion, culture, or growth, including as a substrate for 2D or 3D cell culture and/or tissue growth or tissue engineering.

In one example, the thin film composition is a substrate for cell adhesion, culture, or growth, or a substrate for cell transport or delivery.

In another example, the thin film composition is for packaging.

In various embodiments, the one or more crystallin proteins is a crystallin protein isolated and/or purified from vertebrate eye tissue.

In one embodiment, the one or more crystallin proteins is a recombinant crystallin protein.

In various embodiments, the one or more crystallin proteins is a crystallin protein selected from the group comprising α-crystallin, β-crystallin, γ-crystallin, and a combination of any two or more thereof. In one example, the α-crystallin protein is αA-crystallin. In one example, the α-crystallin protein is αB-crystallin.

In various embodiments, the native secondary structure of the one or more crystallin proteins is maintained, the native tertiary structure of the one or more crystallin proteins is maintained, and/or the native quaternary structure of the one or more crystallin proteins is maintained. For example, the one or more crystallin proteins are substantially free of nanofibrils or other disrupted structural forms.

In various embodiments, at least some of the crystallin protein present in the composition is natively glycosylated—that is, has a glycosylation pattern and degree comparable to that of the same crystallin when present in the organism from which it is derived, isolated, or purified.

In various embodiments, when present—for example in thin film compositions as described herein, the plasticizer is selected from the group comprising polyhydric alcohols, diesters or triesters of acids, diesters or triesters of alcohols, polyethylene glycol, polypropylene glycol, and combinations of any two or more thereof.

In one embodiment, the polyhydric alcohol is selected from the group comprising glycerol, propylene glycol, polyvinyl alcohol, sorbitol, and maltitol.

In one example, the plasticizer is glycerol, sorbitol, or a combination thereof.

In one embodiment, the diester or triester of acid is selected from the group comprising triethyl citrate (TEC), tributyl citrate (TBC), acetyl triethyl citrate (ATEC), dibutyl sebacate (DBS), diethyl phthalate (DEP), and dibutyl phthalate (DBP).

In one embodiment, the diester or triester of alcohol is selected from the group comprising triacetin (TA), vegetable oils, fractionated coconut oil, and acetylated monoglycerides.

In one embodiment, the composition comprises from about 0.5% w/w to about 3% w/w plasticizer. For example, the composition comprises from about 0.5% w/w to about 2.5% w/w plasticizer, from about 1% w/w to about 2.5% w/w plasticizer, from about 1.5% w/w to about 2.5% w/w plasticizer, or about 2% w/w plasticizer.

In various embodiments, when present—for example in thin film compositions as described herein, the co-initiator is a biocompatible tertiary amine based coinitiator, such as TEAOH, L-arginine, and the like.

In one embodiment, the composition comprises from about 0.5% w/w to about 5% w/w co-initiator. For example, the composition comprises from about 0.5% w/w to about 4% w/w co-initiator, from about 1% w/w to about 4% w/w co-initiator, from about 1.5% w/w to about 3% w/w co-initiator, or about 2% w/w co-initiator.

In various embodiments, the crosslinker is selected from the group comprising polyethylene glycol diglycidyl ether (PEGDE), glutaraldehyde, riboflavin, riboflavin-5-monophosphate, polyethylene glycol diacrylate (PEGDA), and any combination of two or more thereof.

In one embodiment, the crosslinker is a photocrosslinker. For example, the photocrosslinker is selected from the group comprising 2-hydroxy-1-[4-(2-hydroxyethoxy) phenyl]-2-methyl-1propanone (Irgacure® 2959); 1-hydroxycyclohexyl-1-phenyl ketone (Irgacure® 184), 2,2dimethoxy-2-phenylacetophenone (Irgacure® 651), riboflavin, lithium phenyl-2,4,6-trimethylbenzoylphosphinate (LAP), eosin Y (EY) and triethanolamine (TEOA), genepin, NHS-EDC, modified PEGs including NHS-PEG, and any combination of two or more thereof.

In one embodiment, the composition comprises from about 0.1% w/w to about 1.5% w/w crosslinker, such as riboflavin or riboflavin-5-monophosphate. For example, the composition comprises from about 0.1% w/w to about 1% w/w crosslinker, from about 0.1% w/w to about 0.8% w/w crosslinker, from about 0.1% w/w to about 0.6% w/w crosslinker, from about 0.1% w/w to about 0.5% w/w crosslinker, from about 0.1% w/w to about 0.4% w/w crosslinker, from about 0.1% w/w to about 0.3% w/w crosslinker, from about 0.1% w/w to about 0.2% w/w crosslinker, or about 0.2% w/w crosslinker.

In one embodiment, the composition comprises from about 0.5% w/w to about 3% w/w crosslinker, such as PEGDE or glutaraldehyde. For example, the composition comprises from about 0.5% w/w to about 2.5% w/w crosslinker, from about 1% w/w to about 3% w/w crosslinker, from about 1% w/w to about 2.5% w/w crosslinker, from about 1% w/w to about 3% w/w crosslinker, from about 1.5% w/w to about 2.5% w/w crosslinker, or about 2.5% w/w crosslinker.

In another embodiment, the composition comprises from about 3% w/w to about 30% w/w crosslinker, such as PEGDA. For example, the composition comprises from about 5% w/w to about 30% w/w crosslinker from about 5% w/w to about 25% w/w crosslinker, from about 10% w/w to about 30% w/w crosslinker, from about 10% w/w to about 25% w/w crosslinker, from about 15% w/w to about 30% w/w crosslinker, from about 15% w/w to about 25% w/w crosslinker, from about 20% w/w to about 25% w/w crosslinker, or about 20% w/w crosslinker.

In various embodiments, the composition comprises from about 10 mg/mL to about 200 mg/mL crystallin protein. For example, the composition comprises from about 30 mg/mL to about 150 mg/mL crystallin protein, from about 30 mg/mL to about 140 mg/mL crystallin protein, from about 30 mg/mL to about 130 mg/mL crystallin protein, from about 30 mg/mL to about 120 mg/mL crystallin protein, from about 30 mg/mL to about 110 mg/mL crystallin protein, from about 30 mg/mL to about 100 mg/mL crystallin protein, from about 30 mg/mL to about 90 mg/mL crystallin protein, from about 30 mg/mL to about 80 mg/mL crystallin protein, from about 30 mg/mL to about 70 mg/mL crystallin protein, or from about 30 mg/mL to about 60 mg/mL crystallin protein.

In various embodiments, for example in embodiments relating to adhesive compositions, the composition comprises from about 30 mg/mL to about 150 mg/mL crystallin protein, for example, from about 30 mg/mL to about 120 mg/mL crystallin protein, from about 30 mg/mL to about 110 mg/mL crystallin protein, from about 50 mg/mL to about 110 mg/mL crystallin protein, from about 50 mg/mL to about 100 mg/mL crystallin protein, from about 50 mg/mL to about 90 mg/mL crystallin protein, from about 50 mg/mL to about 80 mg/mL crystallin protein, from about 50 mg/mL to about 70 mg/mL crystallin protein, from about 50 mg/mL to about 60 mg/mL crystallin protein, or about 60 mg/mL crystallin protein. In other embodiments, for example in embodiments relating to adhesive compositions, the composition comprises from about 40 mg/mL to about 150 mg/mL crystallin protein, for example, from about 50 mg/mL to about 150 mg/mL crystallin protein, from about 60 mg/mL to about 150 mg/mL crystallin protein, from about 70 mg/mL to about 150 mg/mL crystallin protein, from about 80 mg/mL to about 150 mg/mL crystallin protein, from about 80 mg/mL to about 140 mg/mL crystallin protein, from about 80 mg/mL to about 130 mg/mL crystallin protein, from about 80 mg/mL to about 120 mg/mL crystallin protein, from about 90 mg/mL to about 130 mg/mL crystallin protein, from about 90 mg/mL to about 120 mg/mL crystallin protein, from about 100 mg/mL to about 130 mg/mL crystallin protein, from about 100 mg/mL to about 120 mg/mL crystallin protein, from about 110 mg/mL to about 130 mg/mL crystallin protein, from about 110 mg/mL to about 120 mg/mL crystallin protein, or about 120 mg/mL crystallin protein.

In various embodiments, for example in embodiments relating to gel compositions, the composition comprises from about 10 mg/mL to about 120 mg/mL crystallin protein, for example, from about 10 mg/mL to about 110 mg/mL crystallin protein, from about 10 mg/mL to about 100 mg/mL crystallin protein, from about 10 mg/mL to about 90 mg/mL crystallin protein, from about 10 mg/mL to about 80 mg/mL crystallin protein, from about 20 mg/mL to about 80 mg/mL crystallin protein, from about 30 mg/mL to about 80 mg/mL crystallin protein, or from about 40 mg/mL to about 80 mg/mL crystallin protein.

In various embodiments, for example in embodiments relating to film compositions including thin film compositions, the composition comprises from about 50 mg/mL to about 120 mg/mL crystallin protein, for example, from about 50 mg/mL to about 110 mg/mL crystallin protein, from about 50 mg/mL to about 100 mg/mL crystallin protein, from about 50 mg/mL to about 90 mg/mL crystallin protein, from about 50 mg/mL to about 80 mg/mL crystallin protein, from about 50 mg/mL to about 70 mg/mL crystallin protein, from about 50 mg/mL to about 60 mg/mL crystallin protein, or about 60 mg/mL crystallin protein.

In certain particularly contemplated embodiments, such as but not limited to thin film compositions, the composition comprises from about 100 mg/mL to about 120 mg/mL crystallin protein, from about 5 mM to about 10 mM glutaraldehyde, and from about 1.5% w/w to about 2.5% w/w glycerol. For example, the composition comprises about 120 mg/mL crystallin protein, from about 5 mM to about 10 mM glutaraldehyde, and about 2% w/w glycerol.

In one particularly contemplated example, the composition is a thin film composition comprising about 60 mg/mL crystallin, about 2% (v/v) glycerol, and about 2.5% (w/v) PEGDE. In one example, said thin film composition is particularly suited for use in packaging applications.

In another particularly contemplated example, the composition is a thin film composition comprising about 60 mg/mL crystallin, about 2% (v/v) glycerol, and about 5 mM GA. In one example, said thin film composition is particularly suited for use in cell culture applications.

In another particularly contemplated example, the composition is a thin film composition comprising about 60 mg/mL crystallin, about 2% (v/v) glycerol, about 0.2% (w/w crystallin) riboflavin-5-phosphate, and optionally about 0.4% (w/w crystallin) L-arginine.

In certain particularly contemplated embodiments, such as but not limited to adhesive compositions, the composition comprises from about 100 mg/mL to about 120 mg/mL crystallin protein, from about 10% w/w to about 20% w/w PEGDA, and from about 0.2% w/w to about 1.0% w/w photoinitiator, such as Igracure, for example Igracure 2959. For example, the composition comprises about 120 mg/mL crystallin protein, about 15% w/w PEGDA, and about 0.5% w/w Igracure 2959.

In certain particularly contemplated embodiments, the composition comprises from about 50 mg/mL to about 80 mg/mL crystallin protein, from about 10% w/w to about 20% w/w PEGDA, and from about 0.2% w/w to about 1.0% w/w photoinitiator, such as Igracure, for example Igracure 2959. For example, the composition comprises about 60 mg/mL crystallin protein, about 15% w/w PEGDA, and about 0.5% w/w Igracure 2959.

In various embodiments, the composition comprises from about 50 mg/mL to about 80 mg/mL crystallin protein, from about 25% w/w to about 50% w/w PEGDA, from about 0.2% to about 1.0% w/w photoinitiator, such as riboflavin, and from 10% w/w to 20% w/w of co-initiator, such as L-arginine. For example, the composition comprises about 60 mg/mL crystallin protein, about 50% w/w PEGDA, about 0.2% riboflavin, and about 10% L-arginine.

In various embodiments, the crosslinked composition is optically transparent over the visible spectrum. For example, the crosslinked composition has a transmittance of light across the visible spectrum (400 nm to 700 nm) of greater than about 75%, for example, greater than about 80%, or greater than about 85%. It will be appreciate that optical transparency and/or high transmissivity, for example high transmissivity over the visible spectrum, will be advantageous to thin film compositions, to adhesive compositions, and to gel compositions as contemplated herein alike.

In various embodiments, the crosslinked composition has an elastic modulus of from about 1 MPa to about 6 MPa. For example, the crosslinked composition has an elastic modulus of from about 1.5 MPa to about 6 MPa, or from about 1.6 MPa to about 6 MPa, such as from about 1.6 MPa to about 5.6 MPa.

In various embodiments, the crosslinked composition has an Ultimate Tensile Strength (UTS) of from about 0.1 MPa to about 1.5 MPa. For example, the crosslinked composition has a UTS of from about 0.1 MPa to about 1 MPa, or from about 0.3 MPa to about 1 MPa.

In various embodiments, the crosslinked composition has a 0.2% Yield Strength of from about 0.1 MPa to about 1 MPa.

In various embodiments, a thin film formed from a composition as described herein comprising from about 100 mg/mL crystallin protein, from about 5 mM to about 10 mM glutaraldehyde, and about 2% glycerol has:
i) an elastic modulus of from about 10 Mpa to about 20 Mpa;
ii) a UTS of from about 0.5 Mpa to about 1.2 Mpa;
iii) a 0.2% Yield Strength of from about 0.2 Mpa to about 0.8 Mpa
iv) any two or more of i) to iii) above.

In various embodiments, a thin film formed from a composition described herein has:
i) an elastic modulus of from about 0.5 Mpa to about 4.0 Mpa;
ii) a UTS of from about 0.1 Mpa to about 1.0 Mpa;
iii) a 0.2% Yield Strength of from about 0.01 Mpa to about 0.5 Mpa
iv) any two or more of i) to iii) above.

In various embodiments, two or more compositions as described herein are used in combination. For example, in a particularly contemplated embodiment, an adhesive composition as described herein is applied to a thin film formed from a composition as described herein, for example to provide a thin film-adhesive composition suitable for surgical applications such as ocular surgery.

It is intended that reference to a range of numbers disclosed herein (for example, 1 to 10) also incorporates reference to all rational numbers within that range (for example, 1, 1.1, 2, 3, 3.9, 4, 5, 6, 6.5, 7, 8, 9 and 10) and also any range of rational numbers within that range (for example, 2 to 8, 1.5 to 5.5 and 3.1 to 4.7).

Other objects, aspects, features and advantages of the present invention will become apparent from the following description. It should be understood, however, that the detailed description and the specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE FIGURES

FIG. 1 shows an SDS-PAGE of semipurified crystallin samples as described herein in Example 2: a) crystallin extracted using 15 min homogenisation and b) crystallin extracted using 35 min homogenisation, and dialysed. Samples were diluted in 1:100 ratio using a buffer, and Milli-Q respectively before loading on the gel. L=ladder (molecular weights indicated in kDa on the left-hand side), C=crude crystallin extract.

FIG. 2 presents an SDS-PAGE of extracted crystallins from different species, showing the presence of different classes/sub-classes of crystallin proteins, α, β, and γ in all three different sources, where a) Hoki lens, b) Human lens, c) Porcine lens, and CE is the crude extract.

FIG. 3 shows the size exclusion separation of extracted crude crystallin proteins as described in Example 2. Peak fractions correspond to total α (red), β (blue), and γ (green) crystallins.

FIG. 4 is a size exclusion chromatogram of crystallin proteins extracted from different sources, where a) Hoki lens, b) Human lens, and c) Porcine lens, as described in Example 2.

FIG. 5 is an SDS-PAGE of recombinant human α-crystallin as described in Example 2 herein. SDS-PAGE revealed that the size of the purified α-crystallin matched its predicted size of 20 kDa.

FIG. 6 is three amino acid sequence alignments of: (a) αB-crystallin proteins from D. rerio and H. sapiens (b) PA4-crystallin proteins from D. rerio and H. sapiens, and (c) γB-crystallin proteins from H. sapiens, B. taurus, and D. rerio.

FIG. 7 is an amino acid sequence alignment of several vertebrate αA-crystallin orthologues (from Runkle et al., 2002). Asterisks indicate amino acids identical to the zebrafish sequence. Dashes represent gaps introduced to optimize alignment.

FIG. 8 is an amino acid sequence alignment of several vertebrate αB-crystallin orthologues (from Posner et al., 1999). Asterisks indicate amino acids identical to the zebrafish sequence. Dashes represent gaps introduced to optimize alignment.

FIG. 9 is a circular dichroism spectrum of crude crystallin extract from Hoki fish lens as described herein in Example 2. Clearly visible is a minimum centered at 217 nm, indicative of β-sheet structure in crystallins.

FIG. 10 is an FTIR absorbance spectrum of crude crystallin extract from Hoki fish lens, as described in Example 2 herein. A significant peak at 1631 cm⁻¹ is visible, representative of β-sheet structure.

FIG. 11 is a graph depicting the analysis of chaperone-like protection of lysozyme by crystallin extract against TCEP-induced aggregation, as described in Example 3 herein.

FIG. 12 shows representative images of LIVE/DEAD staining of cells to confirm biocompatibility of crude crystallin extract from Hoki fish lens, as described in Example 3 herein. Shown are images of control cells (in the absence of crystallin), and cells grown in the presence of purified α, β, and γ-crystallin fractions (10 mg/mL).

FIG. 13 is a graph showing the impact of crude crystallin extract from Hoki fish lens on cell proliferation, as described in Example 3 herein. Error bar represents the standard deviation of mean taken from 3 set of experiments with triplicate samples.

FIG. 14 is a graph showing the protective effect of crude crystallin extract from Hoki fish lens against oxidative stress, as described in Example 3 herein. Error bar represents the standard deviation of mean taken from 3 set of experiments with triplicate samples.

FIG. 15 is a graph depicting the Upper Tensile Strength (UTS) of thin film compositions, displayed as MPa of force withheld, as described in Example 5. Asterisks denote P-value threshold of 0.05 (*), 0.01 (**), and 0.01 (***).

FIG. 16 is a graph depicting the Young's modulus (Elastic modulus) values of thin film compositions, as described in Example 5 herein. Asterisks denote P-value threshold of 0.05 (*), 0.01 (**), and 0.01 (***).

FIG. 17 is a graph depicting the Extension values of thin film compositions, as described in Example 5 herein. Asterisks denote P-value threshold of 0.05 (*), 0.01 (**), and 0.01 (***).

FIG. 18 is a graph showing the swelling behaviour of crystallin hydrogels obtained via UV curing, as described in Example 5 herein. Error bar represents the standard deviation of mean taken from triplicate samples.

FIG. 19 shows cellular adhesion and outgrowth of cells labelled with anti-Alpha Tubulin and DAPI stain supported by a representative thin film composition as described in Example 6 herein. All scale bars 50 μm with visualisation at 20× in rows I and III, and 63× in rows II and IV. A-F seeded with corneal-scrape cell line 1. G-L seeded with corneal-scrape cell line 2.

FIG. 20 shows DAPI staining of human corneal epithelial cells at P3, as described in Example 6 herein. Film formulations F2 (A-C), F3 (D-F), F4 (G-I), and TCP control (J-L). Time point day 0 (A, D, G, J), day 7 (B, E, H, K), day 14 (C, F, I, L). Image size 14.0 mm×15.0 mm.

FIG. 21 is nine photos showing the biocompatibility of crystallin films, as visualised by DAPI nuclear staining of human corneal epithelial cells, as described in Example 6 herein. Film formulations, Top to bottom F2, F3, and F4. Left to right: Day 0/Adherence, day 7, Day 14. 5× zoom, full tile, 13 mm coverslips shown by solid line, edge of cast film shown by dotted line.

FIG. 22 is three graphs showing the biocompatibility of crystallin films, as described in Example 6 herein. Fold increase in cell numbers on crystallin film, and a tissue culture plastic control at day 0 (adhesion), day 7 and day 14, where Grey—F2, Blue—F3, Dark gray—F4, and Light blue is tissue culture plate.

FIG. 23 is six photos showing light microscopy visualisation of cell adhesion on the same day as seeding on thin film compositions, as described in Example 7 herein. All scale bars 200 μm. Top row, first panel, F1; Top row, second panel, F2; Top row, third panel, F3; Bottom row, first panel, F4; Bottom row, second panel, F5; Bottom row, third panel, F6.

FIG. 24 is four sets of photos visualising cell growth on thin film compositions during prolonged cell culture, as described in Example 8 herein, where (a) F2, (b) F3, (c) F4, and (d) negative glass control. Scale bars=50 um FIG. 25 is four photos showing limbal explant (donor 2) cellular outgrowth on representative thin film formulations, as described in Example 9 herein. Live cell imaging at (A) day 7, (B) day 11 and (C) day 14. (D) Fluorescent visualisation of immunohistochemical labelling of vimentin (Red) and cell nuclei labelled with DAPI on explant culture fixed at 14 days. Scale bars=200 μm.

FIG. 26 is three graphs depicting the optical transparency of crystallin thin films, as described in Example 10 herein. A) F2; B) F3; C) F4.

FIG. 27 is three photos and a graph depicting the optical transparency of crystallin films, as described in Example 10 herein. Image showing transparent films, where (a) F2, (b) F3, and (c) F4; Graph—Transmittance of films when hydrated. Grey triangle—F2, Blue circle—F3, and Dark grey square—F4. Error bar represents the standard deviation of mean taken from six samples

FIG. 28 shows an SDS-PAGE showing PEGylation of crystallin using different PEG derivatives and cross-linkers, as described herein in Example 11: L—Ladder, 1—Crystallin only, 2—Crystallin+PEG diethylene (PEGDE), 3—Crystallin+succinimidyl methylene PEG (smPEG) incubated at 37° C.; 4,5,6-glutaraldehyde was added to sample 1, 2, 3 after 1 hour and incubated for 1 h at room temperature; 8,9,10-glutaraldehyde was added to sample 1, 2, 3 immediately and samples were incubated at 37° C. for 1 h, respectively.

FIG. 29 is a photo demonstrating the transparency of PEGDA+crystallin containing hydrogels, as described in Example 12 herein. Top: PEGDA+irgacure 2959, after 5 min UV exposure; Middle: PEGDA+irgacure 2959+Crystallin at 60 mg/mL, after 5 min UV exposure; Bottom: PEGDA+irgacure 2959+Crystallin at 120 mg/mL, after 5 min UV exposure.

FIG. 30 is a graph depicting the optical transparency of PEGDA based crystallin hydrogels, as described in Example 12 herein.

FIG. 31 is a graph depicting the FTIR analysis of PEGDA based crystallin hydrogels, as described in Example 12 herein, showing PEGDA only (blue), and PEGDA based crystallin hydrogels (black).

FIG. 32 presents a representative image of contact angle measurements to determine the wettability of crystallin films, as described in Example 13 herein.

FIG. 33 is a graph showing the stability of crystallin films, as described in Example 13 herein. Grey—F2, Blue—F3, and Dark gray—F4, where error bars represent the standard deviation of mean taken from six samples.

FIG. 34 is two circular dichroism spectrograms presenting the results of sterilization of crystallin films, as described in Example 13 herein. a) crystallin protein from F2 films, and b) crystallin protein from F3 films, where solid line represents films incubated in milliQ for 24 h, and dotted line is gamma sterilized films incubated in milliQ for 24 h.

FIG. 35 presents representative images of crystallin film (a) stored at room temperature for 3 months, (b) gamma sterilized, (c) hydrated film samples after gamma irradiation, as described in Example 13 herein.

FIG. 36 is two photos depicting the adhesive efficacy of PEGDA based crystallin adhesives, as described in Example 14 herein. (a) Samples after 3 min exposure (i) PEGDA only, (ii) PEGDA based crystallin; (b) Samples were physically stretched (forced) to re-open the incision.

FIG. 37 is three photos presenting the visualisation of optimised formulations for (a) UV curing, (b) visible light curing before and after curing, as described herein in Example 15.

FIG. 38 is two photos showing the adhesive efficacy of PEGDA based crystallin adhesives in a porcine eye adhesion model, as described in Example 15 herein. (a) eye sample with incision, (b) after crystallin hydrogel application and UV cured for 3 min.

FIG. 39 is three photos showing the surgical tractability of crystallin films as established in a suture test using a porcine eye model, as described in Example 15 herein.

FIG. 40 is a photo and a graph presenting data on the adhesive strength of UV cured crystallin bio-adhesive formulation as determined in a lap shear test applied to a porcine skin sample, as described in Example 15 herein. Top: a representative image of porcine skin adhesion sample used in the lap shear test. Bottom: Adhesive strength of a UV cured crystallin bioadhesive formulation compared to a Fibrin glue value taken from literature (Nakayama & Matsuda, 1999). Error bars represent the standard deviation of mean taken from six samples.

FIG. 41 is four photos showing the efficacy of crystallin films as a cell carrier, using decellularised human cornea from limbal explants on F2 carrier film, as described in Example 16 herein, where (a) Cell free control, (b) Cornea placed on top of cultured cells, (c) Cornea placed under cultured cells, and (d) Cornea placed under cultured cells ×10 magnification. Scale Bars=500 μm.

FIG. 42 is four graphs showing drug delivery characteristics of single layer thin films as described in Example 17 herein. A) % drug released in PBS, as measured at 230 nm; B) % drug released in PBS, as measured at 271 nm; C) solution concentration of drug released in PBS, as measured at 230 nm; D) solution concentration of drug released in PBS, as measured at 271 nm.

FIG. 43 is four graphs showing drug delivery characteristics of multi-layer thin films as described in Example 17 herein. A) solution concentration of drug released in PBS, as measured at 230 nm; B) % drug released in PBS, as measured at 230 nm; C) solution concentration of drug released in PBS, as measured at 271 nm; D) % drug released in PBS, as measured at 271 nm.

FIG. 44 is a graph showing drug (tetracycline) release from crystallin hydrogels obtained via UV curing, as described in Example 18 herein. Black line data: cumulative release of tetracycline over 7 days as a percentage of the tetracycline added during polymerization (i.e., fresh gels). Grey line data: the cumulative release of tetracycline over 7 days as a percentage of the tetracycline absorbed into dried hydrogels. Error bar represents the standard deviation of mean taken from triplicate samples.

### DETAILED DESCRIPTION

The present invention relates to biocompatible materials comprising one or more crystallin proteins, and uses thereof, including in a range of therapeutic and scientific methods. For example, the present invention relates to biocompatible compositions, including processes of production of biocompatible compositions, and uses thereof, such as in surgery, in cell-based therapies and methods, and in drug delivery. The present invention thus also relates to the use of one or more crystallin proteins in the preparation of biocompatible compositions, such as biocompatible adhesives, hydrogels, thin films, and implants, including compositions, adhesives, hydrogels, and implants particularly suited to ocular therapies.

In various embodiments, the invention relates to biocompatible composition comprising one or more isolated, purified, recombinant or synthesised protein selected from the group comprising:
a) an α-crystallin;
b) a β-crystallin;
c) a γ-crystallin;
d) a protein from any one of a) to c) above from Hoki (Macruronus novaezelandiae);
e) a protein from any one of a) to c) above from Homo sapiens;
f) a protein comprising the amino acid sequence identified in Table 1 herein;
g) a polypeptide comprising or consisting of at least about 10 contiguous amino acids from any one of a) to f) above;
h) a protein having at least about 90% amino acid identity to any one of a) to g) above;
i) a protein according to any one of a) to h) above having the native structure of a crystallin protein in vivo;
j) any combination of two or more of a) to i) above;

optionally one or more plasticizers; and

one or more crosslinkers.

A wide range of uses for these compositions exist. Nevertheless, as will be apparent to the skilled addressee, the focus of this description is therapeutic and research applications of such compositions.

### Definitions

The terms “adhesive composition” and related terms refer to a composition that is or can form an adhesive capable of binding two or more surfaces or separate items together and that to at least some degree resists separation. Particularly contemplated herein are adhesive compositions for use in surgery, that is, as surgical adhesives.

The term “amino acid” refers to natural amino acids, non-natural amino acids, and amino acid analogues. Unless otherwise indicated, the term “amino acid” includes both D and L stereoisomers if the respective structure allows such stereoisomeric forms.

Natural amino acids include alanine (Ala or A), arginine (Arg or R), asparagine (Asn or N), aspartic acid (Asp or D), cysteine (Cys or C), glutamine (Gin or Q), glutamic acid (Glu or E), glycine (Gly or G), histidine (His or H), isoleucine (He or I), leucine (Leu or L), Lysine (Lys or K), methionine (Met or M), phenylalanine (Phe or F), proline (Pro or P), serine (Ser or S), threonine (Thr or T), tryptophan (Tip or W), tyrosine (Tyr or Y) and valine (Val or V).

Non-natural amino acids include, but are not limited to, azetidinecarboxylic acid, 2-aminoadipic acid, 3-aminoadipic acid, beta-alanine, naphthylalanine (“naph”), aminopropionic acid, 2-aminobutyric acid, 4-aminobutyric acid, 6-aminocaproic acid, 2-aminoheptanoic acid, 2-aminoisobutyric acid, 3-aminoisbutyric acid, 2-aminopimelic acid, tertiary-butylglycine (“tBuG”), 2,4-diaminoisobutyric acid, desmosine, 2,2′-diaminopimelic acid, 2,3-diaminopropionic acid, N-ethyl glycine, N-ethylasparagine, homoproline (“hPro” or “homoP”), hydroxylysine, allo-hydroxylysine, 3-hydroxyproline (“3Hyp”), 4-hydroxyproline (“4Hyp”), isodesmosine, allo-isoleucine, N-methylalanine (“MeAla” or “Nime”), Nalkylglycine (“NAG”) including N-methylglycine, N-methylisoleucine, N-alkylpentylglycine (“NAPG”) including N-methylpentylglycine. N-methylvaline, naphthylalanine, norvaline (“Norval”), norleucine (“Norleu”), octylglycine (“OctG”), ornithine (“Orn”), pentylglycine (“pG” or “PGly”), pipecolic acid, thioproline (“ThioP” or “tPro”), homoLysine (“hLys”), and homoArginine (“hArg”).

The term “amino acid analogue” refers to a natural or non-natural amino acid where one or more of the C-terminal carboxy group, the N-terminal amino group and side-chain functional group has been chemically blocked, reversibly or irreversibly, or otherwise modified to another functional group. For example, aspartic acid-(beta-methyl ester) is an amino acid analogue of aspartic acid; N-ethylglycine is an amino acid analogue of glycine; or alanine carboxamide is an amino acid analogue of alanine. Other amino acid analogues include methionine sulfoxide, methionine sulfone, S-(carboxymethyl)-cysteine, S-(carboxymethyl) cysteine sulfoxide and S-(carboxymethyl)-cysteine sulfone.

As used herein, the term “peptide” refers a short polymer of amino acids linked together by peptide bonds. In contrast to other amino acid polymers (e.g., proteins, polypeptides, etc.), peptides are generally of about 50 amino acids or less in length. A peptide can comprise natural amino acids, non-natural amino acids, amino acid analogues, and/or modified amino acids. A peptide can be a subsequence of naturally occurring protein or a non-natural, including a synthetic, sequence.

As used herein, the term “synthetic peptide” encompasses a peptide having a distinct amino acid sequence from those found in natural peptides and/or proteins. A “synthetic peptide,” as used herein, can be produced or synthesized by any suitable method (e.g., recombinant expression, chemical synthesis, enzymatic synthesis, etc.).

The terms “peptide mimetic” or “peptidomimetic” refer to a peptide-like molecule that emulates a sequence derived from a protein or peptide. A peptide mimetic or peptidomimetic can contain amino acids and/or non-amino acid components. Examples of peptidomimetics include chemically modified peptides, peptoids (side groups are appended to the nitrogen atom of the peptide backbone, rather than to the α-carbons), β-peptides (amino group bonded to the β carbon rather than the α-carbon), etc. Chemical modification includes one or more modifications at amino acid side groups, α-carbon atoms, terminal amine group, or terminal carboxy group. A chemical modification can be adding chemical moieties, creating new bonds, or removing chemical moieties. Modifications at amino acid side groups include, without limitation, acylation of lysine ε-amino groups, N-alkylation of arginine, histidine, or lysine, alkylation of glutamic or aspartic carboxylic acid groups, lactam formation via cyclization of lysine ε-amino groups with glutamic or aspartic acid side group carboxyl groups, hydrocarbon “stapling” (e.g., to stabilize alpha-helix conformations), and deamidation of glutamine or asparagine. Modifications of the terminal amine group include, without limitation, the desamino, N-lower alkyl, N-di-lower alkyl, constrained alkyls (e.g. branched, cyclic, fused, adamantyl) and N-acyl modifications. Modifications of the terminal carboxy group include, without limitation, the amide, lower alkyl amide, constrained alkyls (e.g. branched, cyclic, fused, adamantyl) alkyl, dialkyl amide, and lower alkyl ester modifications. Lower alkyl is C1-C4 alkyl. Furthermore, one or more side groups, or terminal groups, can be protected by protective groups known to the ordinarily skilled peptide chemist. The a-carbon of an amino acid can be mono- or dimethylated.

It will be appreciated that any one of the proteins described herein in certain embodiments comprises one or more non-naturally occurring amino acids, one or more amino acid analogues, or is or comprises a synthetic peptide or polypeptide or a peptide mimetic.

As used in this specification, the words “comprise”, “comprises”, “comprising”, and similar words, are not to be interpreted in an exclusive or exhaustive sense. In other words, they are intended to mean “including, but not limited to”. When interpreting each statement in this specification that includes the term “comprise”, “comprises”, or “comprising”, features other than that or those prefaced by the term may also be present.

A “fragment” of a polypeptide is a subsequence of the polypeptide, typically one that performs a function that is required for activity, such as enzymatic or binding activity, and/or provides a three dimensional structure of the polypeptide or a part thereof, such as an epitope.

The term “fusion polypeptide”, as used herein, refers to a polypeptide comprising two or more amino acid sequences, for example two or more polypeptide domains, fused through respective amino and carboxyl residues by a peptide linkage to form a single continuous polypeptide. It should be understood that the two or more amino acid sequences can either be directly fused or indirectly fused through their respective amino and carboxyl termini through a linker or spacer or an additional polypeptide.

In one embodiment, one of the amino acid sequences comprising the fusion polypeptide comprises a particle-forming protein, and one or more other amino acid sequences comprising the fusion protein comprises a protein as herein described.

A “hydrogel”, in the context of the present disclosure, is taken to mean a water-containing, but itself water-insoluble, polymer, the molecules of which are chemically linked to form a three-dimensional matrix. Owing to the hydrophilic components incorporated, hydrogels swell in water, increasing in volume, without losing in the process their material cohesion.

A “gel” as used herein refers to a solid material capable of at least a degree of deformation while substantially retaining material cohesion.

As used herein, the term “physiological pH” generally refers to a pH that normally prevails in the human body, and ranges from about 7.35 to about 7.4. However, in certain contexts that will be clear to the person skilled in the art, including in the context of preparative methods described herein involving the preparation of crystallin proteins from a sample, physiological pH will refer to the pH that normally prevails in the body of the organism from which the sample was obtained.

As used herein, the term “polypeptide” is intended to encompass a singular “polypeptide” as well as plural “polypeptides,” and refers to a molecule composed of monomers (amino acids) linearly linked by amide bonds (also known as peptide bonds). The term “polypeptide” refers to any chain or chains of two or more amino acids, and does not refer to a specific length of the product. Thus, peptides, dipeptides, tripeptides, oligopeptides, “protein,” “amino acid chain,” or any other term used to refer to a chain or chains of two or more amino acids, are included within the definition of “polypeptide,” and the term “polypeptide” may be used instead of, or interchangeably with any of these terms.

The term “polypeptide” is also intended to refer to the products of post-expression modifications of the polypeptide, including without limitation glycosylation, acetylation, phosphorylation, amidation, derivatization by known protecting/blocking groups, proteolytic cleavage, or modification by non-naturally occurring amino acids. A polypeptide may be derived from a natural biological source or produced by recombinant technology, but is not necessarily translated from a designated nucleic acid sequence. It may be generated in any manner, including by chemical synthesis.

A polypeptide of the invention may be of a size of about 3 or more, 5 or more, 10 or more, 20 or more, 25 or more, 50 or more, 75 or more, 100 or more, 200 or more, 500 or more, 1,000 or more, or 2,000 or more amino acids. Polypeptides may have a defined three-dimensional structure, although they do not necessarily have such structure. The term glycoprotein refers to a protein coupled to at least one carbohydrate moiety that is attached to the protein via an oxygen-containing or a nitrogen-containing side chain of an amino acid residue, e.g., a serine residue or an asparagine residue.

Polypeptides as contemplated herein thus encompass amino acid chains of any length, including full-length proteins, in which amino acid residues are linked by covalent peptide bonds. Polypeptides described herein are purified natural products, or are produced partially or wholly using recombinant or synthetic techniques. The term may refer to a polypeptide, an aggregate of a polypeptide such as a dimer or other multimer, a fusion polypeptide, a polypeptide variant, or derivative thereof.

It will be understood that, for the particular polypeptides and proteins contemplated herein, natural variations can exist between individual species. These variations may be demonstrated by (an) amino acid difference(s) in the overall sequence or by deletions, substitutions, insertions, inversions or additions of (an) amino acid(s) in said sequence. Amino acid substitutions which do not essentially alter biological and immunological activities, are well known. Amino acid replacements between related amino acids or replacements which have occurred frequently in evolution are, inter alia, Ser/Ala, Ser/Gly, Asp/Gly, Asp/Asn, Ile/Val. Other amino add substitutions include Asp/Glu, Thr/Ser, Ala/Gly, Ala/Thr, Ser/Asn, Ala/Val, Thr/Phe, Ala/Pro, Lys/Arg, Leu/Ile, Leu/Val and Ala/Glu. Based on this information, methods for rapid and sensitive protein comparison and determining the functional similarity between homologous proteins were developed. Such amino acid substitutions of the exemplary embodiments described herein, as well as variations having deletions and/or insertions are within the scope of the invention as long as the resulting proteins retain their immune reactivity. This explains why one or more proteins described herein, when isolated from different sources, may have identity levels below 100%, while still representing the same protein with the same characteristics. Those variations in the amino acid sequence of a certain protein described herein that still provide a functional protein, such as a protein capable of reacting with an antibody specific to a protein specifically identified herein, are considered as functional equivalents of the proteins identified herein, and as such do not essentially influence the functionality of the protein.

By an “isolated” polypeptide or a fragment, variant, or derivative thereof is intended a polypeptide that is not in its natural milieu. No particular level of purification is required. For example, an isolated polypeptide can be removed from its native or natural environment. Recombinantly produced polypeptides and proteins expressed in host cells are considered isolated for purposed of the invention, as are native or recombinant polypeptides which have been at least partially separated, fractionated, or partially or substantially purified by any suitable technique.

The term “substantially pure” used in reference to a polypeptide (or fragment, variant, or derivative thereof) refers to polypeptides as described herein that are separated as desired from RNA, DNA, proteins or other contaminants with which they are naturally associated. For example, when referring to proteins and polypeptides, a protein or polypeptide is considered substantially pure when that protein makes up greater than about 50% of the total protein content of the composition containing that protein, and typically, greater than about 60% of the total protein content. More typically, a substantially pure or isolated protein or polypeptide will make up at least about 75%, at least about 80%, at least about 85%, more preferably, at least about 90%, at least about 95% of the total protein. Preferably, the protein will make up greater than about 90%, and more preferably, greater than about 95% of the total protein in the composition. It will be appreciated that modern methods of recombinantly producing or of synthesising proteins and polypeptides are well suited to producing substantially pure polypeptides.

The term “variant” with reference to polypeptides encompasses naturally occurring, recombinantly, and synthetically produced polypeptides, including those comprising one or more non-natural amino acids, one or more amino acid analogues, and peptide mimetics. Variant polypeptide sequences preferably exhibit at least 50%, more preferably at least 51%, at least 52%, at least 53%, at least 54%, at least 55%, at least 56%, at least 57%, at least 58%, at least 59%, at least 60%, at least 61%, at least 62%, at least 63%, at least 64%, at least 65%, at least 66%, at least 67%, at least 68%, at least 69%, at least 70%, at least 71%, at least 72%, at least 73%, at least 74%, at least 75%, at least 76%, at least %, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity to a sequences of the present invention. Identity is found over a comparison window of at least 20 amino acid positions, preferably at least 50 amino acid positions, at least 100 amino acid positions, or over the entire length of a polypeptide of the invention.

Polypeptide sequence identity can be determined in the following manner. The subject polypeptide sequence is compared to a candidate polypeptide sequence using BLASTP (from the BLAST suite of programs, version 2.2.10 [October 2004]) in bl2seq, which is publicly available from NCBI (ftp://ftp.ncbi.nih.gov/blast/). The default parameters of bl2seq are utilized except that filtering of low complexity regions should be turned off.

Polypeptide sequence identity may also be calculated over the entire length of the overlap between a candidate and subject polynucleotide sequences using global sequence alignment programs. EMBOSS-needle (available at http:/www.ebi.ac.uk/emboss/align/) and GAP (Huang, X. (1994) On Global Sequence Alignment. Computer Applications in the Biosciences 10, 227-235.) as discussed above are also suitable global sequence alignment programs for calculating polypeptide sequence identity.

Polypeptide variants contemplated herein also encompass those which exhibit a similarity to one or more of the specifically identified sequences that is likely to preserve the functional equivalence of those sequences and which could not reasonably be expected to have occurred by random chance. Such sequence similarity with respect to polypeptides can be determined using the publicly available bl2seq program from the BLAST suite of programs (version 2.2.10 [October 2004]) from NCBI (ftp://ftp.ncbi.nih.gov/blast/). The similarity of polypeptide sequences can be examined using the following unix command line parameters:

bl2seq -i peptideseq1 -j peptideseq2 -F F -p blastp

Variant polypeptide sequences preferably exhibit an E value of less than 1×10⁻¹⁰, more preferably less than 1×10⁻²⁰, less than 1×10⁻³⁰, less than 1×10⁻⁴⁰, less than 1×10⁻⁵⁰, less than 1×10⁻⁶⁰, less than 1×10⁻⁷⁰, less than 1×10⁻⁸⁰, less than 1×10⁻⁹⁰, less than 1×10⁻¹⁰⁰, less than 1×10⁻¹¹⁰, less than 1×10⁻¹²⁰ or less than 1×10⁻¹²³ when compared with any one of the specifically identified sequences.

The parameter -F F turns off filtering of low complexity sections. The parameter -p selects the appropriate algorithm for the pair of sequences. This program finds regions of similarity between the sequences and for each such region reports an “E value” which is the expected number of times one could expect to see such a match by chance in a database of a fixed reference size containing random sequences. For small E values, much less than one, this is approximately the probability of such a random match.

Conservative substitutions of one or several amino acids of a described polypeptide sequence without significantly altering its biological activity are also included in the invention. A skilled artisan will be aware of methods for making phenotypically silent amino acid substitutions (see, e.g., Bowie et al., 1990, Science 247, 1306).

A polypeptide variant contemplated herein also encompasses that which is produced from the nucleic acid encoding a polypeptide, but differs from the wild type polypeptide in that it is processed differently such that it has an altered amino acid sequence. For example, a variant is produced by an alternative splicing pattern of the primary RNA transcript to that which produces a wild type polypeptide.

A “subject” as used herein is an animal, usually a mammal, including a mammalian companion animal or a human. Representative companion animals include feline, equine, and canine. Representative agricultural animals include bovine, ovine, caprine, cervine, and porcine. Specifically contemplated subjects are subjects which are used commercially to produce milk, such as bovine, ovine, and caprine subjects.

The term “vector” refers to a polynucleotide molecule, usually double stranded DNA, which is used to transport the genetic construct into a host cell. In certain examples the vector is capable of replication in at least one additional host system, such as E. coli.

Crystallin proteins Crystallin proteins are water soluble structural proteins found in the eye lens of all vertebrate species. Crystallins function to maintain the required refractive index of the lens, and comprise ˜90% or more of the protein component of an eye lens fibre cell.

Crystallin proteins found in the lens and cornea of the eye can be divided into three subgroups, α-crystallin, β-crystallin and γ-crystallin. The proportion of each subgroup present in eye tissue differs by species: approximately 35% of protein in a typical mammalian lens is α-crystallin; in fish and rodents, the proportion of γ-crystallins is greater than β-crystallins; and the major component in most other species' lenses is β-crystallin.

α-Crystallin

α-Crystallin is a member of the small heat-shock protein (sHSP) group of proteins. sHSPs all contain a distinctive α-crystallin domain comprising about 90 amino acids, with a hydrophobic N-terminal domain and hydrophilic C-terminal extension. There are two α-crystallin subunits, αA and αB. The polydisperse and oligomeric nature of the αA and αB crystallins means their size is dependant on the environment. Their average homooligomeric molecular weights have been found to be 660 kDa and 620 kDa for αA and αB, respectively, however the oligomer may be in the range of 300 to 1200 kDa.

It has been reported that α-crystallin in the lens helps prevent the aggregation of denatured proteins that form cataracts, and increases cells' tolerance to stress.

β-Crystallin and γ-Crystallin

β- and γ-crystallins are structurally similar proteins, both composed of two similar domains where each domain has two similar motifs folded in the Greek key pattern. γ-crystallin is a simple monomer, while β-crystallin is a complex group of oligomers. Both β- and γ-crystallins have been found in tissues outside the lens, although their specific biological functions are poorly characterised.

Specifically contemplated crystallin proteins are mammalian α-crystallins, such as αA crystallin or αB crystallins, mammalian β-crystallins, such as βA crystallins (e.g., βA1, βA2, βA3, and βA4 crystallins), and βB crystallins (e.g., βB1, βB2, and βB3 crystallins), and mammalian γ-crystallins, such as γS, γA, γB, γC, γD, γE, and γF crystallins.

Further specifically contemplated crystallin proteins are piscine α-crystallins, such as αA crystallins or αB crystallins, piscine β-crystallins, such as βA crystallins (e.g., βA1, βA2, and βA4 crystallins), and PB crystallins (e.g., βB1, βB2, and βB3 crystallins), and piscine γ-crystallins, such as γM1, γM3, γM4, γM5, γM7, γM8a, γM8b, γM8c, γ8d, γM8e, γM9, γN, γS1, and γS2 crystallins from, for example, Antarctic toothfish (Dissostichus mawsoni), or γM1, γM2a, γM2b, γM2c, γM2d1, γM2d2, γM3, γM4, γM5, γM6, γM7, γMx, γN1, γN2, γS1, γS2, γS3, and γS4 crystallins from Zebrafish (Danio rerio).

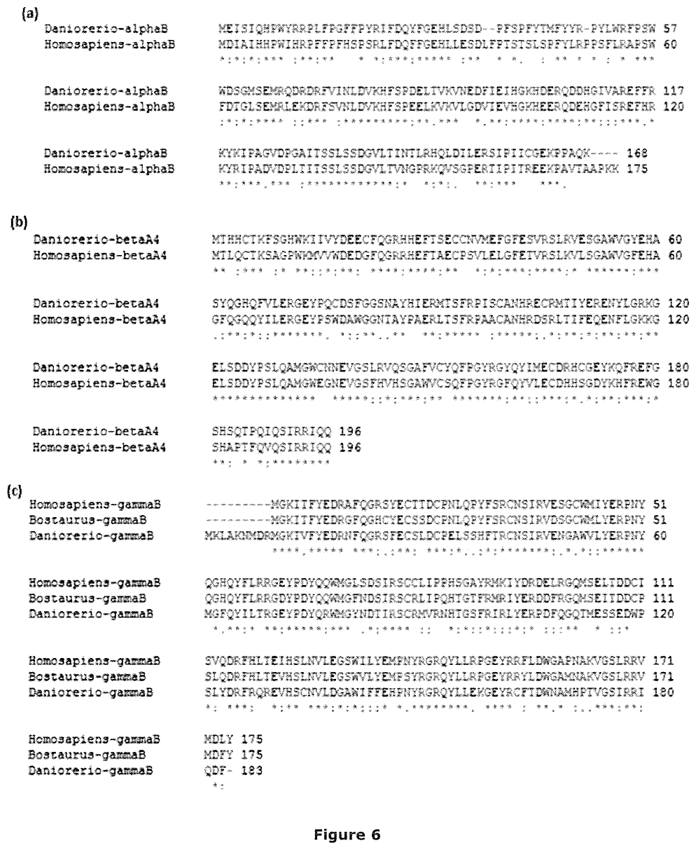

Still further specifically contemplated crystallin proteins from Homo sapiens are set out in Table 1 below and in FIGS. 6, 7, and 8.

TABLE 1
Homo sapiens crystallin proteins

| Crystallin | Genbank ref | Crystallin | Genbank ref | Crystallin | Genbank ref |
|---|---|---|---|---|---|
| α-A | NP_001300979.1 | β-A2 | NP_476434.1 | γ-A | NP_055432.2 |
| α-B1 | NP_001276736.1 | β-A3 | NP_005199.2 | γ-C | NP_066269.1 |
| α-B | AAP36581.1 | β-A4 | NP_001877.1 | γ-D | NP_008822.2 |
| | | β-B1 | NP_001878.1 | γ-N | NP_653328.1 |
| | | β-B2 | NP_000487.1 | γ-S | NP_060011.1 |
| | | β-B3 | NP_004067.1 | | |
| | | β-S | XP_018879280.1 | | |

It will be appreciated that while crystallin proteins purified from naturally-occuring sources are specifically exemplified herein, recombinant or synthetic crystallin proteins are likewise amenable to use in the methods, compositions, and materials described herein. It will further be appreciated that, irrespective of the source of the crystallin proteins, it is believed, without wishing to be bound by any theory, that certain of the advantageous properties of the compositions and materials described herein relate to the native conformation of the crystallin proteins when present in the compositions and materials. Accordingly, production methods capable of providing crystallin proteins having their native conformation, structure, and modification (including post-translational modifications such as glycosylation patterns), are specifically contemplated. Nucleic acids, constructs, vectors, and host cells capable of expressing or producing crystallin proteins, including recombinant or synthetic crystallin proteins, are thus particularly contemplated herein.

Those skilled in the art will recognise on reading this description that various uses of and for compositions comprising crystallin proteins, particularly in therapeutic methods including surgery, cell therapies, and drug delivery, are provided.

Particularly contemplated herein are therapeutic methods, compositions, reagents, and kits that utilise one or more of the protein-containing compositions as described herein in surgical applications.

Accordingly, in one aspect the invention relates to a method of tissue closure in a subject in need thereof, the method comprising

optionally applying force to close the laceration, lesion, incision or wound;

contacting a laceration, lesion, incision, or wound or the site of said laceration, lesion, incision, or wound with a crystallin protein containing composition as herein described,

optionally applying force to close the laceration, lesion, incision or wound,

initiating crosslinking;

maintaining the closure of the laceration, lesion, incision or wound for a time sufficient for crosslinking to occur;

wherein crosslinking of the crystallin proteins forms an adhesive composition.

In one embodiment, the method of tissue closure is a method of closing a surgical incision.

In one embodiment, the method of tissue closure is a method of sutureless closure. For example, the sutureless closure is sutureless skin closure, sutureless wound closure, or sutureless operative incision closure.

In one embodiment, the surgery is ophthalmic surgery. In one example, the ophthalmic surgery is cataract surgery, conjunctival grafts, vitrectomy including pars planar vitrectomy, refractive lens exchange, lens implantation, or lens replacement surgery. In another example, the ophthalmic surgery is retinal detachment surgery, including retinal surgery incorporating retinopexy or scleral buckling, macular hole surgery, conjunctival closures, glaucoma surgery, bleb leak surgery, trabeculectomy, blepharorrhaphy, amniotic membrane transplantation, corneal perforation surgery, pterygium surgery including pterygium excision, posterior capsule intraocular lens implantation, epithelial ingrowth surgery, keratoplasty including lamellar keratoplasty, deep anterior lamellar keratoplasty, strabismus surgery including bilateral strabismus surgery, eyelid skin graft surgery, or mucous membrane graft surgery.

In one embodiment, the composition is applied via an ophthalmic surgical device.

In various embodiments, maintenance of the closure of the laceration, lesion, incision or wound is for a time sufficient for crosslinking to occur is by the application of one or more medical aids, such as bandages, sutures, meshes or the like, or by (usually temporary) physical force, such as clamping or holding the laceration, lesion, incision or wound closed.

In various embodiments, maintenance of the closure of the laceration, lesion, incision or wound is for a time sufficient for greater than about 60% crosslinking to occur, for example, greater than about 70% crosslinking to occur, greater than about 80% crosslinking to occur, greater than about 90% crosslinking to occur, or greater than about 95% crosslinking to occur.

For the avoidance of doubt, as used herein reference to a percentage of crosslinking contemplates that proportion of the total available crosslinking sites present in the crystallin protein which have formed a crosslink and are thus involved in crosslinking. It will be appreciated that, while effective adhesion can be achieved using compositions as herein described when less that complete crosslinking has occurred, it is desirable to allow a substantial proportion of those crosslinks that can be formed to be formed to provide robust adhesion. Similarly, those skilled in the art will recognise that the force required to achieve and maintain tissue closure, and therefore the degree of crosslinking desired (for example during the maintenance step of representative surgical methods described herein), depends on a number of factors, not least the site, extent, depth and/or area of the laceration, lesion, incision, or wound, the age and motility of the subject, and the availability of or desirability for using other medical aids, such as bandages, surgical meshes, sutures, and the like, or physical force to aid tissue closure.

In some embodiments, the crosslinking time (or gelling time) of the biocompatible crystallin-comprising material is controlled by the pH, for example, the pH of the composition, the pH of the target site, the pH of an aqueous buffer, or the like.

In some embodiments, the crosslinking time is controlled by initiation of crosslinking, for example, exposure of a photocrosslinker to light, such as UV light.

In certain embodiments, the crosslinking time is between about 20 seconds and 10 minutes. In some embodiments, the biocompatible crystallin-comprising material gels at a target site. In certain embodiments, the biocompatible crystallin-comprising material gels at a predetermined time.

In some embodiments, the biocompatible crystallin-comprising material is a bioabsorbable polymer. In certain embodiments, the biocompatible crystallin-comprising material is bioabsorbed within about 1 to 70 days.

In some embodiments, the biocompatible crystallin-comprising material is substantially non-bioabsorbable.

In most surgical applications involving the use of compositions contemplated herein as surgical adhesives or that are at leas partially reliant on or benefit from the adhesive capability of compositions as contemplated herein, crosslinking of the composition is advantageously carried out and/or initiated once the composition has been applied or administered. However, it will be recognised that in certain embodiments, the use of an at least partially crosslinked composition is beneficial, such that continued crosslinking after application or administration is usefully maintained.

Accordingly, in one aspect, the invention relates to a method of tissue closure in a subject in need thereof, the method comprising

optionally applying force to close the laceration, lesion, incision or wound;

contacting a laceration, lesion, incision, or wound or the site of said laceration, lesion, incision, or wound with a crystallin protein containing composition as herein described, optionally wherein the crystallin containing composition is at least partially crosslinked,

optionally applying force to close the laceration, lesion, incision or wound,

initiating and/or maintaining crosslinking;

maintaining the closure of the laceration, lesion, incision or wound for a time sufficient for crosslinking to occur;

wherein application and/or crosslinking of the crystallin proteins forms an adhesive composition.

Broadly equivalent methods of tissue closure in a subject who is undergoing or who has undergone ophthalmic surgery, and of treating an ocular injury or ocular incision in a subject in need thereof, are also contemplated, as described herein.

It will be appreciated that in many applications of the compositions, uses and methods described herein, the ability of the crystallin-comprising compositions to undergo sterilization and retain efficacy, including useful structural integrity and function, is of significant importance. Those skilled in the art will recognise on reading this disclosure that sterility is of paramount importance in many contemplated applications, such as in surgical procedures, and procedures involving the culturing or transfer of cells. Representative examples of suitable sterilization methods are exemplified herein, such as for example gamma irradiation (see Example 13) UV sterilization (see Example 4).

It will be recognised that sterilization of the compositions will typically be performed when it is most expedient to do so, in light of the use to which the composition is to be put and how it is to be or has been handled, stored, transported, administered, and the like. In certain examples, sterilization is performed after the crystallin-comprising composition has been crosslinked. For example, in certain embodiments thin film compositions for cell culture or transfer are sterilized after they have crosslinked. Representative methods of post-crosslinking sterilization are exemplified herein.

In certain embodiments, sterilization is at least partially achieved during preparation or use of the compositions contemplated herein. For example, certain UV-cured compositions described herein can be at least partially UV sterilized during curing/crosslinking. For example, exposing a composition suitable for UV-curing to UV light under sterile conditions (see for example those described in Example 4 herein) and for a duration sufficient to crosslink the crystallin proteins and sterilize the composition is particularly contemplated.

In certain embodiments, the composition is sterilized prior to crosslinking. For example, certain uses of adhesive crystallin-comprising compositions described herein involve the topical or surgical application of a composition prior to crosslinking, wherein the composition is advantageously sterile to avoid the introduction of infection to the administration site.

Sterilization by irradiation, including but not limited to gamma irradiation, will generally be preferred, particularly for applications involving surgical administration of the crystallin-comprising compositions contemplated herein. UV sterilisation, particularly of but not limited to UV-curable compositions described herein, is also specifically contemplated.

Chemical sterilization methods, particularly those suitable for the sterilization of temperature- and moisture-sensitive medical devices including implantable medical devices, such as for example ethylene oxide processing (such as treatment with CFC-12/EtO 88/12 blend), are also suitable for use with the compositions described herein. Examples include those listed in the US Environmental Protection Agency's ‘Significant New Alternatives Policy—Substitutes in Sterilants’ website at epa.org/snap/substitutes-sterilants.

Ideally, sterilization is done using methods and under conditions that have no or minimal impact on the efficacy (such as, but not limited to any one or more of formation, structure, or function) of the composition and/or final crosslinked product. Methods to determine the impact, or lack thereof, of sterilization on the compositions described herein, including on the structure of the crystallin protein present in the composition, are described and exemplified herein. For instance, Example 13 herein exemplifies methods to investigate the secondary structure of crystallin proteins present in compositions that had undergone gamma sterilization, to confirm that gamma sterilization had no adverse impact on the native structure of crystallin proteins in films. These and other methods known to those skilled in the art are appropriate to investigate the suitability of other methods of sterilization to ensure sterilization has no or minimal impact on efficacy.

Mechanical Characteristics of Gelled/Crosslinked Compositions

As will be apparent to those skilled in the art on reading this disclosure, the gelled/crosslinked compositions described herein can be characterised by their mechanical properties. For example, the tensile elasticity of compositions described herein is usefully quantified by determining the Young's modulus/Elastic Modulus of the material, wherein the higher the modulus, the stiffer the material. Methods to determine tensile elasticity are well known in the art, and representative methods are described herein in the Examples.

Similarly, the maximum stress a material can withstand before breaking is represented herein as the Ultimate Tensile Strength (UTS), and the point at which the material starts to exhibit permanent/plastic deformation is represented herein as the 0.2% Yield Strength. Generally in design applications the yield strength is used as the upper limit for allowable stress. Again, methods to determine UTS and 0.2% Yield Strength are well known in the art, and representative methods are presented herein in the Examples.

It will be appreciated that the mechanical characteristics of the gelled/crosslinked compositions will in certain embodiments be adapted to particularly suit the application to which the composition(s) are to be put. For example, compositions for use as thin films will in certain embodiments be formulated to have a higher Young's modulus than compositions for use as an adhesive.

It will further be appreciated that the mechanical characteristics of the gelled/crosslinked composition relate at least in part to the formulation of the precursor composition, for example the identity and amount of the crosslinker, and/or the presence or relative amount of a particular crystallin isoform in the precursor composition. As can clearly be seen in the Examples presented herein, altering the composition of the crystallin containing compositions described herein has a meaningful impact on the characteristics of the resulting gelled/crosslinked compositions.

Furthermore, in addition to affecting the mechanical characteristics of the gelled/crosslinked compositions, the formulation of the precursor composition has an influence of other properties of the gelled/crosslinked compositions, such as the release profile for one or more active agents present in the gelled/crosslinked compositions, and/or the degradation rate and/or profile of the composition.

In certain embodiments, for example embodiments relating to thin films, patterning, for example micropatterning of films using soft lithography, or patterning by gelling over a patterned template substrate, is employed. For example, patterning is employed to promote beneficial cell alignment or anchoring. In one embodiment, etched or patterned films, for example films prepared over etched silicon wafers or polyurethane surfaces having 400-4000 nm pitches, are employed to direct the alignment and migration of cells, or to promote cellular adhesion and/or stratification, and/or protein deposition. Similarly, patterning of compositions comprising crystallin proteins as described herein, such as the thin films described herein, can be employed to vary or augment one or more mechanical characteristics, such as elasticity, strength, or to direct deformation, perforation, or other disruption.

In addition to one or more crystallin proteins, the biocompatible compositions contemplated herein may comprise one or more active agents, such as one or more additional polypeptides including one or more synthetic peptides, or one or more therapeutic active agents. For example, a biocompatible material contemplated herein will in certain embodiments comprise one or more substances covalently bound to or incorporated or adsorbed into the material, such as one or more substances bound to one or more of the crystallin polypeptides, or a moiety bound thereto.

In the majority of therapeutic uses, when one or more active agent is present, said active agent will preferably be a physiologically or pharmacologically active agent, such as an agent selected from the group comprising: an antibiotic, a cytostatic, an anti-inflammatory, a metabolism hormone, agents for gene therapy, growth hormones, differentiation or modulation factors, immunosuppressants, immunostimulating substances, nucleic acids, apoptosis-inducing agents, adhesion-inducing or inhibiting agents, receptor agonists and receptor antagonists, or mixtures or any two or more thereof.

Specifically contemplated are compositions, uses and methods relating to ophthalmic therapies, where one or more of the one or more active agents is an ophthalmically acceptable antibiotic, for example, one or more antibiotics selected from the group comprising sulphonamides, macrolides, erhthromycin, chloramphenicol, aminoglycosides, fluoroquinolones, vancomycin, and tetracyclines.

It will be appreciated that a wide range of pharmaceutically-active active agents are amenable to incorporation into biomaterials as described herein, and will be selected in accordance with the therapeutic goals to be achieved, such as the condition or disease to be treated. For example, in the context of ocular therapies particularly contemplated herein, one or more active agents effective to treat, for example, glaucoma, will in certain embodiments be incorporated into biomaterials suitable for application to the eye. Representative active agents for the topical treatment of glaucoma include, but are not limited to, cholinergic agents, such as pilocarpine, carbachol, demecarium bromide and echothiophate iodide; adrenergic agonists, such as epinephrine, dipivefrin, brimonidine and apraclonidine; beta blockers, such as timolol, carteolol, betaxolol, levobunolol and metoprolol; prostaglandin analogues, such as PGF2α, latanoprost, unoprostone and PHXA-85; and carbonic anhydrase inhibitors, such as dorzolamide and brinzolamide.

It will be appreciated that, while the ability to provide localised concentrations of active agent to a particular site is of benefit in many therapeutic applications, the compositions and materials described herein are also suitable for delivery of systemic active agents, particularly when the site of application has good access to the systemic circulation, or otherwise is amenable to active agent uptake, as are the mucosal tissues. Using the example of the eye as contemplated herein, it will be appreciated that systemic delivery of active agents via the eye is possible, such that compositions and materials described herein targeted for application in or on the eye are not limited to the delivery of topical ophthalmic agents, but can comprise one or more systemically active agents.

In certain embodiments, such as certain embodiments exemplified herein, the biocompatible compositions contemplated herein comprise one or more cells, optionally together with one or more supportive active agents and/or one or more additional active agents as discussed above. For example, in certain embodiments, representative compositions for surgical use comprise one or more cells, such as one or more stem cells, together with one or more antibiotics and/or one or more differentiation or modulation factors to improve cell viability before, during and after surgical application.

Particularly contemplated cells include limbal stem cells, which are also referred to as limbal epithelial stem cells or corneal-limbal stem cells, stomal stem cells, which are also referred to as mesenchymal stem/stromal cells, and retinal pigment epithelial cells. Further particularly contemplated cells include totipotent stem cells, pluripotent stem cells, and multipotent stem cells.

The active agents, substances, and cells contemplated herein for use in conjunction with the biocompatible compositions will usually be utilised so as to provide a therapeutic benefit, but as will be appreciated support functions are likewise specifically contemplated.

In certain embodiments, the compositions contemplated herein comprise one or more carriers or excipients, such as one or more diluents or one or more additional agents or substances which provide one or more benefits to the composition and/or its use and/or that of any one or more of its constituents, including any one or more of the active agents also present. For example, the compositions in certain embodiments comprise one or more carriers or excipients which provide one or more benefits in or of the formulation, stability, administration, delivery, uptake, or efficacy of the composition and/or one or more of the one or more active agents comprised therein.

Control of Release Rate of an Active Agent

In some embodiments, the biocompatible crystallin-comprising material slowly delivers an active agent to a target site by diffusion and/or osmosis over time ranging from hours to days. In certain embodiments, the agent is delivered directly to the target site. In some embodiments, the procedure of delivering a biocompatible crystallin-comprising material comprising an active agent to a target site is repeated several times, if needed. In other embodiments, the active agent is released from the biocompatible crystallin-comprising material through biodegradation of the material. In some embodiments, the active agent is released through a combination of diffusion, osmosis, and/or degradation mechanisms. In certain embodiments, the release profile of the active agent from the material is unimodal. In some embodiments, the release profile of the active agent from the material is bimodal. In certain embodiments, the release profile of the active agent from the material is multimodal.

In some embodiments, the active agent is released from the biocompatible crystallin-comprising material though diffusion or osmosis. In certain embodiments, the active agent is substantially released from the biocompatible crystallin-comprising material within 180 days. In some embodiments, the active agent is substantially released from the biocompatible crystallin-comprising material within 14 days. In certain embodiments, the active agent is substantially released from the biocompatible crystallin-comprising material within 24 hours. In some embodiments, the active agent is substantially released from the biocompatible crystallin-comprising material within one hour.

In certain embodiments, the active agent is substantially released from the biocompatible crystallin-comprising material within about 180 days, about 150 days, about 120 days, about 90 days, about 80 days, about 70 days, about 60 days, about 50 days, about 40 days, about 35 days, about 30 days, about 28 days, about 21 days, about 14 days, about 10 days, about 7 days, about 6 days, about 5 days, about 4 days, about 3 days, about 2 days, about 1 day, about 0.5 day, about 6 hours, about 4 hours, about 2 hours, about or 1 hour. In some embodiments, the active agent is substantially released from the biocompatible crystallin-comprising material within more than 180 days, more than 150 days, more than 120 days, more than 90 days, more than 80 days, more than 70 days, more than 60 days, more than 50 days, more than 40 days, more than 35 days, more than 30 days, more than 28 days, more than 21 days, more than 14 days, more than 10 days, more than 7 days, more than 6 days, more than 5 days, more than 4 days, more than 3 days, more than 2 days, more than 1 day, more than 0.5 day, more than 6 hours, more than 4 hours, more than 2 hours, more than or 1 hour. In certain embodiments, the active agent is substantially released from the biocompatible crystallin-comprising material within less than 180 days, less than 150 days, less than 120 days, less than 90 days, less than 80 days, less than 70 days, less than 60 days, less than 50 days, less than 40 days, less than 35 days, less than 30 days, less than 28 days, less than 21 days, less than 14 days, less than 10 days, less than 7 days, less than 6 days, less than 5 days, less than 4 days, less than 3 days, less than 2 days, less than 1 day, less than 0.5 day, less than 6 hours, less than 4 hours, less than 2 hours, less than or 1 hour. In some embodiments, the active agent is substantially released from the biocompatible crystallin-comprising material within about one day to about fourteen days. In certain embodiments, the active agent is substantially released from the biocompatible crystallin-comprising material within about one day to about 70 days.

In some embodiments, the active agent is a biomolecule and the release of the biomolecule from the material is controlled by the composition of the material. In certain embodiments, the biomolecule is released when the material starts to degrade.

In some embodiments, the active agent is a cell or population thereof and the release of the cell or population from the material is controlled by the composition of the material.

In some embodiments, the biocompatible material comprises an active agent, wherein the active agent is released from the biocompatible crystallin-comprising material through diffusion, osmosis, degradation of the biocompatible crystallin-comprising material, or any combination thereof. In certain embodiments, the active agent is initially released from the biocompatible crystallin-comprising material through diffusion and later released through degradation of the biocompatible crystallin-comprising material.

In some embodiments, the active agent is substantially released from the biocompatible crystallin-comprising material within 180 days.

In certain embodiments, the active agent is substantially released from the biocompatible crystallin-comprising material within 24 hours.

In some embodiments, the biocompatible crystallin-comprising material interacts with or is bound to the active agent. In certain examples, more than 10% of the active agent is released through degradation of the biocompatible crystallin-comprising material.

In certain embodiments, the release of the active agent is determined by the composition of the biocompatible crystallin-comprising material. In certain embodiments, the release of the active agent is essentially inhibited until a time that the biocompatible crystallin-comprising material starts to degrade.

In certain embodiments, the time the biocompatible crystallin-comprising material starts to degrade is longer the higher a degree of cross-linking of the biocompatible crystallin-comprising material.

In some embodiments, the active agent is a pharmaceutically active biomolecule. In certain embodiments, the pharmaceutically active biomolecule is a protein, enzyme, or peptide. In some embodiments, the pharmaceutically active biomolecule is an antibody.

In certain embodiments, the pharmaceutically active biomolecule is a vaccine. In some embodiments, the pharmaceutically active biomolecule is an oligonucleotide.

Exemplary Kits and Methods

In one embodiment, a surgical kit is provided comprising a crosslinked biocompatible crystallin-comprising composition as herein described, optionally together with instructions for delivering the crosslinked biocompatible crystallin-comprising composition to a target site, optionally together with a device for delivering the crosslinked biocompatible crystallin-comprising composition to a target site.

Further provided herein is a kit comprising a) a composition comprising a crystallin protein as herein described; and b) a crosslinker; wherein a biocompatible crystallin-comprising material is formed following mixing the composition and the crosslinker.

Also provided here is a kit comprising a) a composition comprising a crystallin protein as herein described; b) a plasticizer, and c) a crosslinker; wherein a biocompatible crystallin-comprising material is formed following mixing the composition, the plasticizer, and the crosslinker.

Also provided here is a kit comprising a) a composition comprising a crystallin protein as herein described, optionally together with a plasticizer; and b) a crosslinker; wherein a biocompatible crystallin-comprising material is formed following mixing the composition and the crosslinker.

Further provided herein is a kit for preparing an in vivo gelling or crosslinking pharmaceutical composition as described herein, comprising a first container with a composition comprising a crystallin protein as herein described, optionally comprising a plasticizer, a second container with a crosslinker, optionally one or more additional containers with one or more active agents, optionally a container with a buffer, optionally a mixing vessel, instructions for mixing the materials present in each container in the mixing vessel and/or instructions for crosslinking to produce the biocompatible crystallin-comprising material, and instructions for delivering the biocompatible crystallin-comprising material to a target site.

Also provided herein is a kit for preparing an in vivo gelling or crosslinking pharmaceutical composition as described herein, comprising a first container with a biocompatible crystallin-comprising composition as herein described, optionally comprising a plasticizer, optionally an additional container with crosslinker, optionally one or more additional containers with one or more active agents, optionally a container with a buffer, optionally a mixing vessel, instructions for mixing the materials present in each container in the mixing vessel and/or instructions for crosslinking to produce the biocompatible crystallin-comprising material, and instructions for delivering the biocompatible crystallin-comprising material to a target site.

In various embodiments, one or more of the kits described herein additionally comprises co-initiator, such as a container comprising co-initiator.

In certain applications of the materials and methods described herein, the in situ formation of the crosslinked material is targeted. In certain embodiments, the in situ crosslinking of the compositions described herein is of an adhesive composition, thereby to provide adhesive efficacy. Such in situ crosslinking is exemplified herein in the Examples.

In other embodiments, compositions used for in situ crosslinking provide a hydrogel material. Here it is advantageous that the composition is not crosslinked to completion until it has been introduced into the body of the patient at the site that is to be protected.

The composition in this case can be used in injectable form or in spray-able form, wherein it is preferably used in a minimally invasive manner, although it is also possible to use it in connection with a surgical intervention.

In certain embodiments, the composition in this case is mixed with a crosslinker, for example immediately before application thereof into the body, and then this mixture is either introduced into the body of the patient as a liquid or as a spray, in such a manner that the crosslinking proceeds only in situ. However, it is also envisaged to conduct the composition and the crosslinker separately to the site in the body of the human or animal patient that is to be protected, and to mix them there. It is further envisaged, for example when photocrosslinkers are used, to conduct the composition comprising the crosslinker to the target site, whereupon crosslinking is initiated by exposure to light of the appropriate wavelength.

Compositions for in situ use, particularly those where crosslinking occurs on addition of the crosslinker without a further initiation step (such as photoactivation), will in certain embodiments be generated immediately before surgical or minimally invasive application and then used as a spray, as an implant, as a liquid, as a plug or as a gel film.

In embodiments where crosslinking needs no further initiation beyond admixture with the crystallin protein comprising composition, the material is therefore introduced onto or into the body of a patient after all the components for its production are present. In such cases, the material crosslinks to completion either before application, during application or else after application.

In embodiments involving implantation of a completely crosslinked material, such as a thin film or hydrogel, a somewhat solid consistency of the material is preferred which permits or facilitates practical handling of the material. The degree of solidity or fluid properties of the material in this case can be set via by the degree of crosslinking, wherein the material is the more solid the more it is crosslinked, or in the context of a thin film, the degree of drying may also contribute to solidity. The fluid properties of a gel are thus between those of a liquid and those of a solid.

Accordingly, the present invention also relates to a kit having a first container which comprises the described composition and having a second container which comprises a crosslinker for the composition for use in the in situ generation of a crystallin protein comprising material, such as an adhesive material, a hydrogel-forming material, or a thin film material.

Those skilled in the art, with the guidance of this disclosure and the Examples provided herein, will recognises that the composition and the crosslinker can be matched to one another in such a manner that the crosslinked biocompatible crystallin-comprising composition suitable for the respective desired treatment is formed.

In this case the rate of crosslinking, the viscosity, the resorption kinetics, and the like, can be adjusted in such a manner that the components to be applied, for example the components present in a kit as herein contemplated, are separately or jointly sprayable or injectable as a liquid.

It will be appreciated that one advantage of such use is that compositions as described herein can be applied, for example in a liquid state, to traumatized and intact tissue surfaces or in tissues which are surgically cleared or incised.

For example, compositions as described herein are in certain embodiments readily used without problems in a minimally invasive manner, for example as a liquid or as a spray. In certain embodiments, the resultant gel or adhesive adapts or adheres to tissues, including any non-uniform tissue surfaces. Particularly contemplated embodiments are amenable to formation on dry tissues, and on moist tissue surfaces without significant running, alike.

As a result, very thin layers can be formed on the tissue surfaces, as even layer thicknesses of less than 1 mm of certain embodiments of the compositions provided herein are sufficiently robust to maintain structural integrity and/or cohesion.

In certain embodiments, when desired the biocompatible is rapidly resorbed, for example within a residence time of less than about 21 days, for example, less than about 14 days.

It will be understood that in most cases, the therapeutic uses contemplated herein will typically require compositions and materials having a very high degree of biocompatibility, and which and do not trigger inflammation, scarring, pathological or undesired tissue formation, pathological or undesired angiogenesis, or pathological or undesired neurogenesis.

Additionally, particularly contemplated biocompatible materials comprising crystallin proteins as described herein, particularly those for surgical use, are robust and simple to handle, either because they can be applied before or during crosslinking while in a predominantly liquid state, for example by injection or spraying, or can be applied when crosslinking is complete, for example as a gel or thin layer. For spraying or injection, when necessary, for example to initiate crosslinking, the components can be combined shortly before or at the site in the body that is to be protected, or else distally to this site.

Generally, the compositions and materials as herein described are suitable for use in surgical contexts without a need for suturing or other incision or wound retention means, thereby to minimise scarring, aberrant tissue formation, and other complications.

The invention is further described with reference to the following examples. It will be appreciated that the invention as claimed is not intended to be limited in any way by these examples.

### EXAMPLES

### Example 1: Preparation of Purified Native Crystallin Proteins

This example describes the preparation of purified crystallin protein compositions in which the native structure of the crystallin proteins is retained.

### Materials and Methods

Fresh human corneoscleral rims were obtained with ethics approval from the New Zealand National Eye Bank. Primary corneal epithelial cell lines utilised were historically derived and stored in liquid nitrogen from tissue also sourced from the New Zealand National Eye Bank. Human amniotic membrane was sourced from the New Zealand National Eye Bank.

Hoki fish heads were obtained from a commercial fishery. Lenses were removed in-house and aliquoted into approximately 12 g lots in 15 mL Falcon tubes and stored at −20° C. Porcine eyes were obtained from a commercial supplier. For extraction and characterisation of crystallins, lenses were processed in the same way as lenses from Hoki.

Solutions and Media

Unless otherwise specified, Milli-Q water used had the resistivity of 18.2MΩ·cm⁻¹, and had been autoclaved prior to use. Filtered Milli-Q was syringe filtered through a 0.20 μm cellulose acetate membrane (GVS Filter Technology, FJ13ASCCA002DL01).

TABLE 2
Solution and media compositions.

| Solution | Components |
|---|---|
| Phosphate Buffered Saline | Phosphate Buffer Saline (PBS) Tablets (MP Biomedicals, |
| (PBS) | 092810305), Milli-Q |
| | Autoclaved prior to use. |
| Crystallin Extraction Buffer | 50 mM Tris(hydroxymethyl)aminomethane (Thermo Fisher |
| | Scientific, BP152-1), 100 mM sodium chloride (NaCl) (Thermo |
| | Fisher Scientific, 207790010), 0.04% sodium azide (NaN3) |
| | (Scharlau, SO0091), filtered Milli-Q. |
| | Adjusted to pH 7.4 using 1M Hydrochloric acid. |
| BSA Standard | Bovine serum albumin (BSA) (MP Biomedicals, ABFF-100G), Milli-Q. |
| Bio-Rad Protein Assay | 1:5 Bio-Rad Protein Assay Dye Reagent Concentrate (Bio-Rad |
| | Laboratories, 5000006), Milli-Q. |
| PEGDE | 50% w/v Poly(ethylene glycol) diglycidyl ether (Sigma-Aldrich, |
| | 475696), PBS. |
| Glutaraldehyde | 200 mM Glutaraldehyde solution (Sigma-Aldrich, G7651), Milli-Q. |
| Riboflavin-5- | 10 mg/mL Riboflavin 5′-monophosphate sodium salt hydrate |
| monophosphate | (Sigma Aldrich, F2253-25MG), Milli-Q. |
| L-Arginine | 5 mg/mL L-Arginine (Sigma-Aldrich, A5006), Milli-Q. |
| Glycerol | 60% w/v Glycerol (Sigma-Aldrich, G5516), Milli-Q. |
| RGD | 1 mg/mL Arg-Gly-Asp (RGD) (Sigma Aldrich, A8052-5MG), PBS. |
| Paraformaldehyde | 4% Paraformaldehyde (PFA) (Sigma-Aldrich, PG148-500G), PBS. |
| Serum Blocking for | 100 mM Glycine (Sigma, G8898-1KG), 0.1% Triton X-100 (37240- |
| Immunohistochemistry | 500G), 10% normal goat serum (NGS) (Thermo Fisher Scientific, |
| | 50197Z) |
| Epithelial Cell Medium | Gibco MEM 1x + GlutaMAX (Gibco, 41090036), Heat inactivated |
| | Fetal Calf Serum (FCS) (10091148), Anti-Anti (15240062) |
| Explant Medium | Gibco DMEM/F12 1x + GlutaMAX (Gibco, 10565018) Heat |
| | inactivated Fetal Calf Serum (FCS) (10091148), Anti-Anti |
| | (15240062), Insulin-transferrin-selenium (ITS)(I3146), Epidermal |
| | growth factor (EGF)(Abacus ALS) |

Lens Crystallin Extraction

For lens crystallin extraction, weighed aliquots of eye lens were thawed and placed into the homogeniser container (IKA ULTRA-TURRAX Tube Drive Workstation). Crystallin extraction buffer was added at a ratio of 2 mL buffer per gram of lens. The homogeniser was then run in 5-minute intervals interspersed with 5-minute chilling phases on ice until the lens solids were dispersed (approximately 30 min). The resulting frothy solution was poured into 50 mL Falcon tubes. Any remaining large, insoluble lens pieces were removed at this point. The crystallin solution was then centrifuged at 4122×g for 30 minutes at 4° C. The supernatant was then distributed to 1.5 mL Eppendorf tubes, and centrifuged again at 9600× g for 30 minutes. The resulting crystallin supernatant was decanted into a clean Falcon tube.

To remove the extraction buffer, 5% v/v glycerol was added to the crystallin solution before injection into a Thermo Scientific Slide-A-Lyzer® dialysis cassette to manufacturer's instructions. Dialysis was undertaken in fresh Milli-Q H₂O for 4 hours at 4° C. with gentle stirring. Approximately 2 L of Milli-Q H₂O was used per cassette, and changed out for fresh every hour. Once completed, the solution was retrieved from the cassette and aliquoted into 50 mL Falcon tubes with final fluid volumes of 15 mL or below. A Bradford assay was run to determine the concentration and expected yield before the solution was lyophilised (Christ Alpha 2-4 LD plus, John Morris Scientific). The lyophilised crystallin was stored at −20° C. until use.

Typical yield of the crystallin extraction process is 36-48%, calculated as follows: approximately 1 g of starting material (e.g., Hoki eye lens) after homogenisation and centrifugation provides 2 mL of crystallin protein extract having a crystallin protein concentration in the range of 180-240 mg/mL (i.e., 360-480 mg total crystallin protein).

### Example 2: Characterisation of Purified and Recombinant Crystallin Proteins

This example describes the characterisation of crystallin proteins prepared as described in Example 1 above.

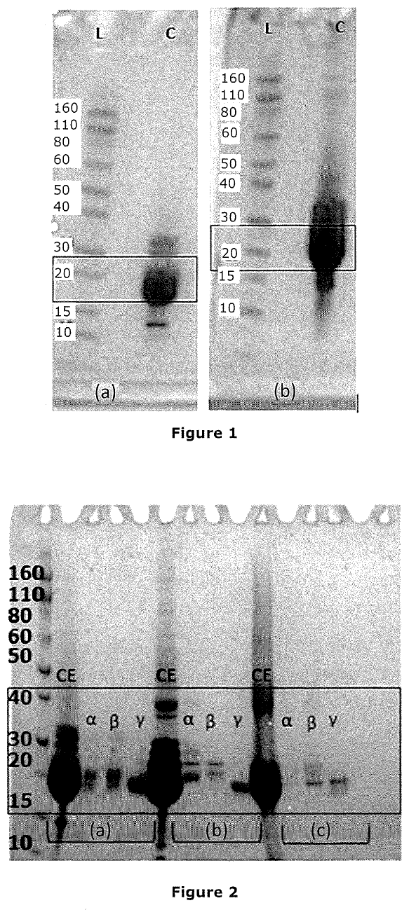

The extracted protein samples were assessed for the presence of crystallin protein by SDS-PAGE. A representative SDS-PAGE from Hoki lens samples is shown in FIG. 1 and in FIG. 2a. There are three distinct classes of crystallin proteins, referred to as α, β, and γ, with each of these classes having distinct subunits. The α-crystallin complex is a highly heterogeneous aggregate of 20 kDa subunits, resulting in multimers of approximately 300 to 1000 kDa. β-Crystallin exists as smaller complexes of approximately 50-200 kDa, formed from 20-30 kDa subunits, and γ-crystallins exist as monomers of approximately 20 kDa (Ecroyd and Carver, 2009). As expected, the SDS-PAGE confirmed the presence of all three classes of crystallin proteins as the extracted sample is not purified further. These three classes of crystallin proteins were also observed in extracts from human lens (FIG. 2b) and porcine lens (FIG. 2c),

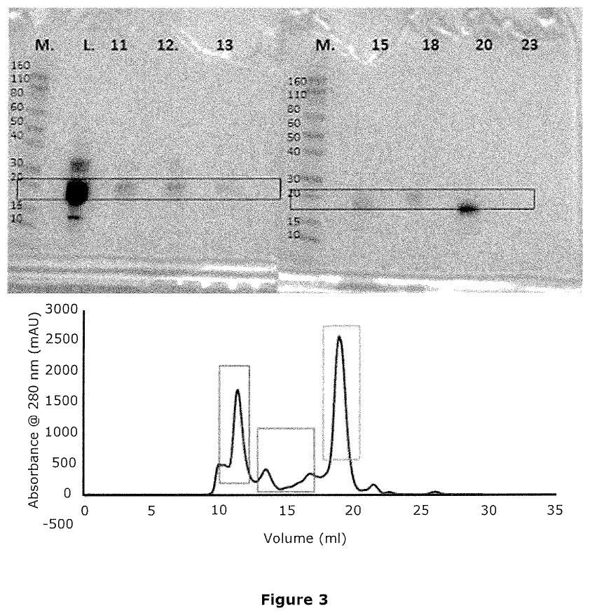

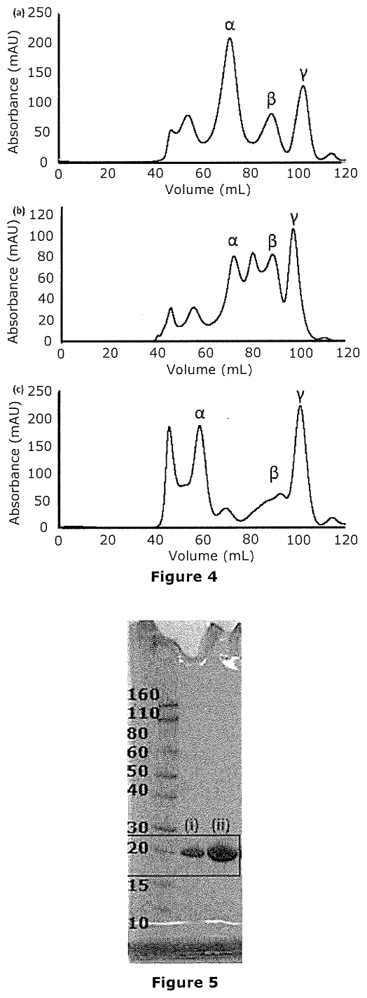

Following SDS-PAGE analysis, the extracted crystallin proteins from Hoki were further semi-purified into the three classes, α, β, and γ, using size exclusion chromatography (SEC), as shown in FIG. 3. Peak fractions corresponding to total α crystallins, total β crystallins, and total γ crystallins, are identified in red, blue, and green boxes, respectively. Further SEC analysis of crystallin proteins extracted from Hoki lens (FIG. 4a), human lens FIG. 4b), and porcine lens (FIG. 4c) again showed the separation of the α-, β-, and γ-crystallin peaks.

Recombinant human α-crystallin was expressed and purified (as described in Horwitz et al, 1998), then characterised by SDS-PAGE as shown in FIG. 5, which revealed that the size of the purified α-crystallin matched its predicted size of 20 kDa.

FIGS. 6, 7 and 8 herein show various amino acid sequence alignments of crystallin proteins from various species, clearly depicting the degree of similarity between these proteins. FIG. 6a is an amino acid sequence alignment of αB-crystallin proteins from D. rerio and H. sapiens, FIG. 6b is an amino acid sequence alignment of βA4-crystallin proteins from D. rerio and H. sapiens, and FIG. 6c is an amino acid sequence alignment of γB-crystallin proteins from H. sapiens, B. taurus, and D. rerio.

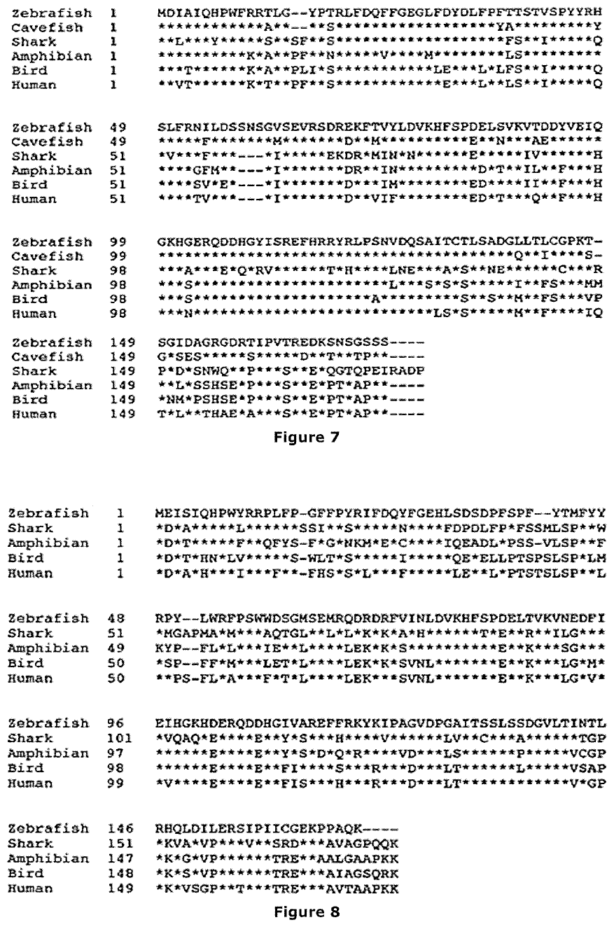

FIG. 7 is an amino acid sequence alignment of several vertebrate αA-crystallin orthologues (from Runkle et al., 2002, adapted from: Integrative and Comparative Biology, Volume 43, Issue 4, August 2003, Pages 481-491, https://doi.org/10.1093/icb/43.4.481). Residues 64-141 of the zebrafish protein correspond to the α-crystallin domain.

FIG. 8 is an amino acid sequence alignment of several vertebrate αB-crystallin orthologues (from Posner et al., 1999, adapted from: Integrative and Comparative Biology, Volume 43, Issue 4, August 2003, Pages 481-491, https://doi.org/10.1093/icb/43.4.481).

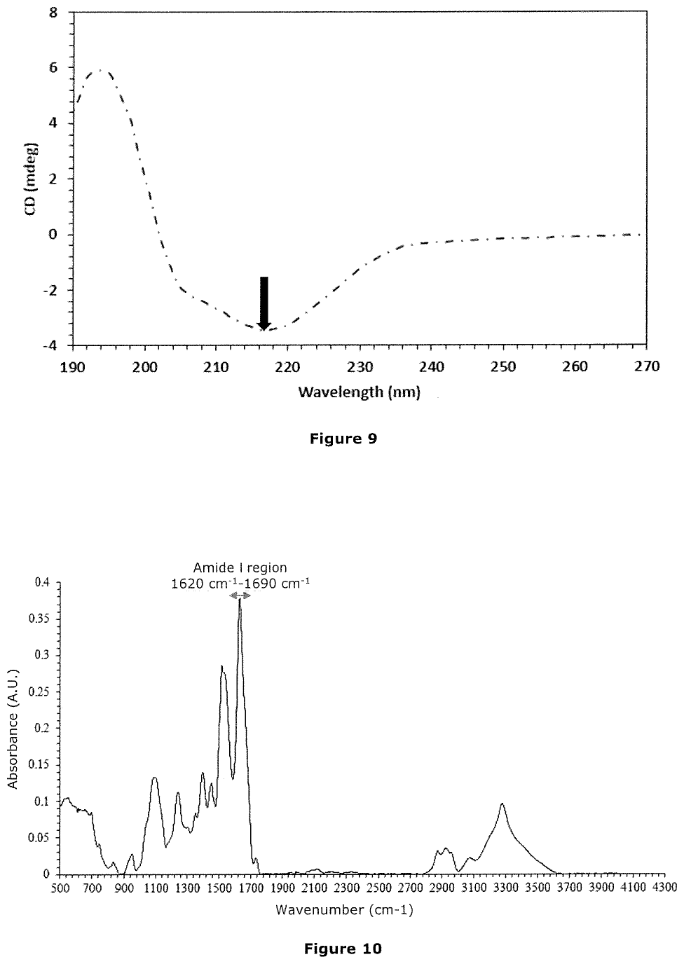

To further characterise the purified crystallin proteins from Hoki lens extract, Circular dichroism (CD) and Fourier-transform infrared (FTIR) spectroscopic methods were employed to investigate protein structure. As can be seen in FIG. 9, a CD spectrogram of crude crystallin extract from Hoki fish lens showed a minimum centered at 217 nm, indicative of β-sheet structure in crystallins. This is consistent with previous reports in literature for bovine crystallins, bovine and human α-crystallins, and toothfish γ crystallins. The significant peak at 1631 cm⁻¹, representative of β-sheet structure, observed with FTIR spectroscopy as shown in FIG. 10 further confirmed the native structure of the crystallin proteins was preserved after extraction and purification using the methods described above.

### Example 3: Functional Characterisation of Purified Crystallin Proteins

This example describes the functional characterisation of crystallin proteins prepared as described in Example 1 above.

The chaperone-like anti-aggregation functionality of certain crystallin proteins was investigated. Briefly, protection by Hoki crystallin extract against TCEP-induced aggregation of lysozyme was assessed, whereby lysozyme (10 μM) was combined with 10 mg/mL crystallin extract obtained from Hoki fish lens and incubated at 37° C. Solutions were monitored for changes in light scattering by measuring absorbance at 400 nm.

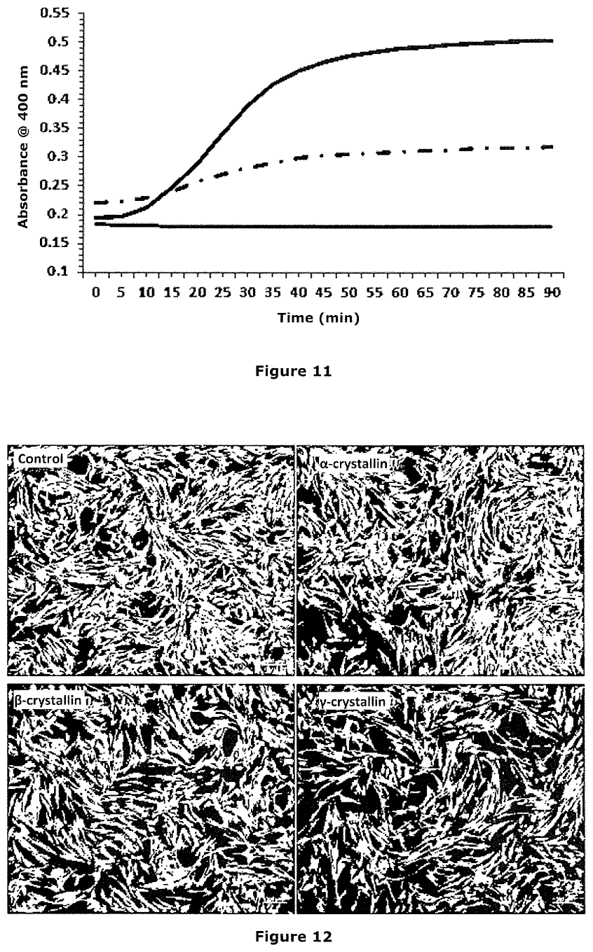

The observed decrease in light scattering in the presence of crystallin (FIG. 11, dotted black) indicated protection of lysozyme from aggregation compared to lysozyme+TCEP in the absence of Hoki crystallin extract (FIG. 11, solid black). Lysozyme only control showed no light scattering in the absence of TCEP (FIG. 11, grey solid).

The biocompatibility with and effect of purified crystallin proteins on mammalian cells was then investigated. First, to establish that crystallin extract was biocompatible with mammalian cells, human corneal epithelial cells were cultured in the presence of purified α-, β-, and γ-crystallin fractions (10 mg/mL) from Hoki lens extract. As shown in the representative images of LIVE/DEAD staining of cells depicted in FIG. 12, the presence of crystallins had no adverse impact on the viability of cells when compared to control (i.e., in the absence of any crystallin).

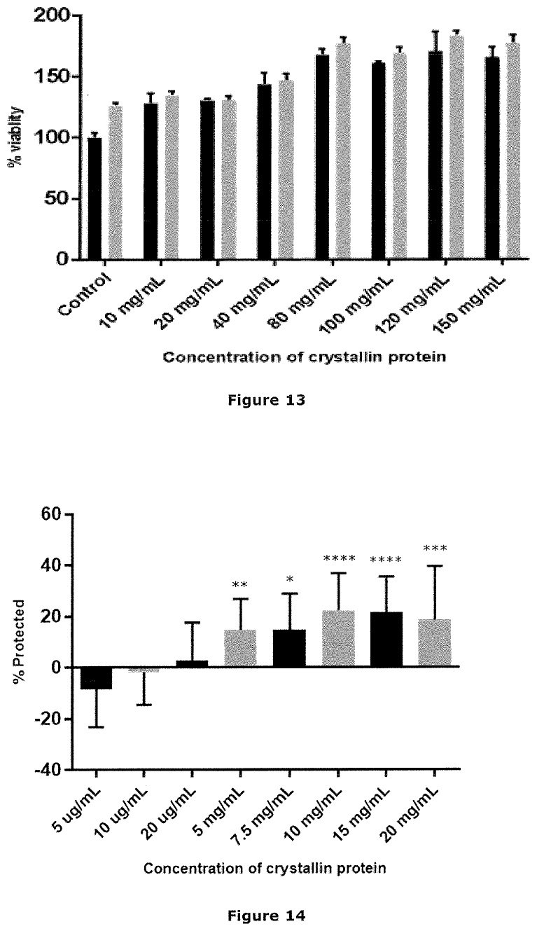

Crude crystallin extract was shown to have a positive impact on mammalian cell proliferation. Human corneal epithelial cells were treated with Hoki crystallin protein for 24 hours in the presence of serum-free media, or media supplemented with 10% FCS, followed by MTT assay. In both cases, as shown in FIG. 13, increased concentrations of crystallin protein resulted in greater cell proliferation.

The crude crystallin extract from Hoki fish lens was then shown to protect mammalian cells from biological stress. In an assay of cellular response to oxidative stress, human corneal epithelial cells were treated with 10 μM H₂O₂, followed by incubation with crystallin. As presented in FIG. 14, MTT assay results showed that higher concentrations (5-20 mg/mL) of crude crystallin extract resulted in an observable increase in cell viability when exposed to H₂O₂, thus establishing a protective effect of crude crystallin extract against oxidative stress.

The work reported in this example clearly demonstrated that crystallin protein prepared using the methods described herein retains not only its native structure, but biologically important functionality as well.

### Example 4: Preparation of Thin Film Compositions Comprising Crystallin Proteins

This example describes the preparation of thin film compositions comprising crystallin proteins.

Film casting was undertaken in a Class 2 hood to maintain sterility. Lyophilised crystallin prepared as described above in Example 1 was resuspended in filter sterilized Milli-Q water. Component volumes (as set out in Table 3 below) were added in the following order to a sterile Eppendorf; Crystallin stock, glycerol, water, additives, cross linker.

TABLE 3
Hoki fish lens protein film formulations

| | Crystallin | Glycerol | | |
|---|---|---|---|---|
| Formulation | (mg/mL) | (%) | Crosslinker | Additives |
| F1 | 60 | 2 | 2.5% w/v PEGDE | |
| F2 | 60 | 2 | 50 mM | |
| | | | Glutaraldehyde | |
| F3 | 60 | 2 | 0.20% w/w R5P* | 0.40% w/w |
| | | | | L-Arginine |
| F4 | 60 | 2 | 0.20% w/w R5P | |
| F5 | 60 | 2 | 2.5% w/v PEGDE | 100 μg/mL |
| | | | | RGD |
| F6 | 60 | 2 | 20% w/v PEGDA | |

*R5P is Riboflavin-5-monophosphate

To ensure consistent crosslinking conditions, each crosslinker was only added to the solution directly prior to that solution's casting. To mix properly, the Eppendorf was inverted ˜10 times.

For films used in cell culture, 50 μL of the solution was cast onto 13 mm glass coverslips and spread to the edges using a pipette tip. Care was taken not to spill the solution over the edges of the coverslip, as the capillary action would draw it underneath and firmly adhere the coverslip to the casting dish. Films were dried in a 37° C. dry oven for 48 hours. The riboflavin films were UV treated for 30 minutes under the hood sterilization UV lights before oven drying.

Films were preferably used immediately after the 48 hour drying period, else stored at room temperature in the casting dish sealed with parafilm.

### Example 5: Functional Characterisation of Thin Film Compositions Comprising Crystallin Proteins

This example describes an assessment of the mechanical characteristics of thin film compositions comprising crystallin proteins.

### Methods

The films used for this testing were cast at 3 mL onto 38 mm circular PDMS moulds. Following casting and UV crosslinking (where appropriate), the films were dried at room temperature for 24 hours, followed by 48 hours drying at 37° C. Testing was undertaken immediately after the allotted drying time was completed.

For testing, the circular films were cut square using a scalpel, with care taken to ensure the largest amount of material possible remained. A micrometer (Mitutoyo) was used to take thickness measurements in the four corners and centre of the film, which was averaged for use in later calculations. The samples were then cut into strips using a 5 mm wide template, and their true final width taken with vernier calipers (Mitutoyo). For each film type 4 strips were obtained and tested.

Testing was undertaken on an Instron 5544 using a 10 N load cell and an extension rate of 10 mm/min. The gauge length was set to 10 mm, and sand paper was placed on the clamps to prevent the samples slipping. The dry film strips were tested until failure.

A failure at the clamps rather than the middle of the film is generally considered a null result, however 100% of samples failed in this way during testing. It was chosen to continue with the results, although it must be noted that due to this failure mode, all suffer from a systematic underrepresentation of strength.

Once the data was acquired, the average thickness and individual strip widths were used to find the cross-sectional area (A) of each sample. Using the load data (F) presented by the Instron at each recorded timepoint, the stress (a) applied to the material at that point was calculated, where σ=F/A.

The extension was also recorded at each timepoint, taken as the change in length (ΔL) of the sample. When divided by the initial gauge length (Li), the percentage of strain (ε %) in the sample was calculated by

$$( ɛ \% ) = \frac{Δ L}{{L}_{i}} \times 100$$

The upper tensile strength (UTS) of the sample can be found by dividing the maximum load applied in newtons by the cross-sectional area.

$$UTS = \frac{{F}_{Max}}{A}$$

The Young's modulus of a material represents stiffness, and can be determined by finding the slope of the stress-strain relationship of a sample during the elastic phase.

### Results

38 mm film castings were made onto PDMS moulds using 3 mL of a solution and a total drying time of 72 hours. The resulting films were flexible, smooth to the touch, and transparent.

Prior to preparation of the films into 5 mm wide strips, the thickness of the films was determined using vernier calipers (Mitutoyo). Following processing the true widths of the processed samples measured to ensure accurate measures of the cross-sectional area were used in UTS calculations. There was variation in the thickness of the films, measured in the centre and 4 corners, likely resulting from inconsistences in the surface of the mould which did not sit perfectly flat.

TABLE 4
Thickness measurements of film used in mechanical testing.

| | Thickness Measurement (mm) | | | | | |
|---|---|---|---|---|---|---|
| Film formulation | 1 | 2 | 3 | 4 | 5 | Average |
| F2 | 0.2 | 0.21 | 0.3 | 0.21 | 0.34 | 0.252 |
| F3 | 0.21 | 0.24 | 0.3 | 0.2 | 0.2 | 0.23 |
| F4 | 0.2 | 0.24 | 0.21 | 0.26 | 0.22 | 0.226 |
| F5 | 0.2 | 0.3 | 0.3 | 0.3 | 0.31 | 0.282 |

During the testing of these compositions, all of the samples failed at the clamps. This is classically considered a failed result, as it is not the force being applied longitudinally that caused the material to fail, but a combination of tensile force and damage inflicted by the clamping process. Therefore, the upper tensile strength scores of the thin films reported here are a systematic underrepresentation of film strength.

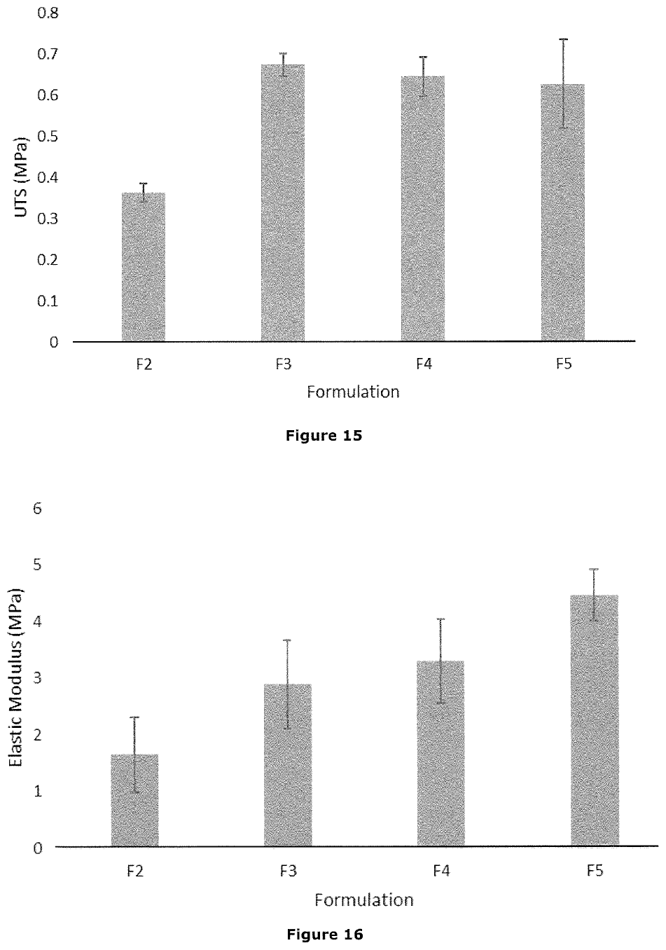

As can be seen in FIG. 15, F2 had the lowest UTS at 0.363±0.0213 MPa, and proved to be the most elastic with a Young's modulus score of 1.65±0.663 MPa (FIG. 16). F3 had an UTS of 0.673±0.0272 MPa and a Young's modulus of 2.89±0.780 MPa. F4 had a UTS and modulus of 0.644±0.04785 MPa and 3.30±0.735 MPa, respectively.

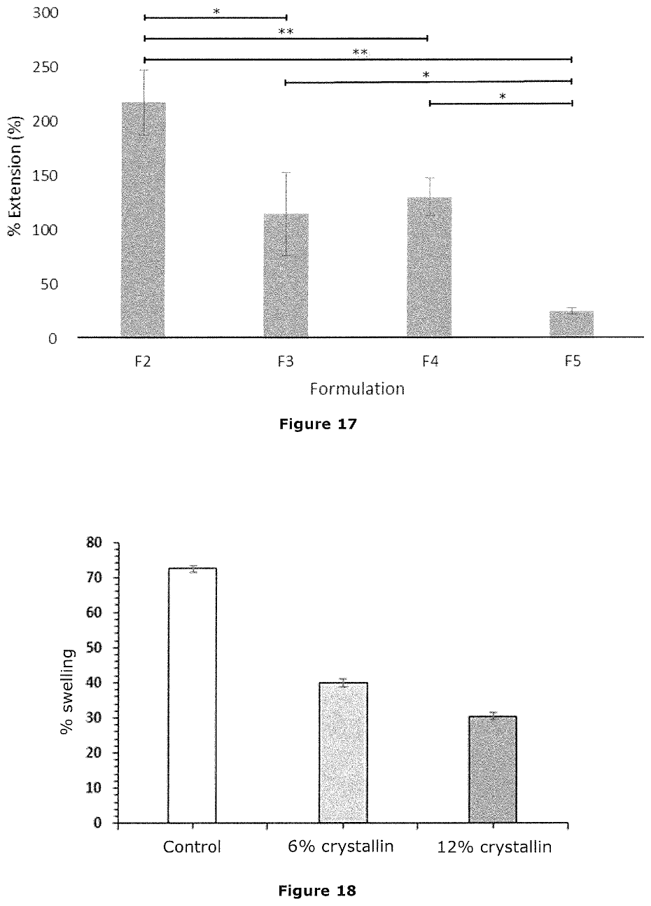

The least elastic of the formulations was F5 with a modulus score of 4.46±0.455 MPa. This score is within the standard deviation of the reported preterm amnion elasticity (3.60±1.4 MPa hydrated (Benson-Martin et al., 2006)), suggesting that these films are either equivalent or more elastic than the current gold standard carrier. F5 had a UTS of 0.626±0.108 MPa, comparable to that of F3 and F4. Extension values for the four compositions were calculated, with each of F2, F3 and F4 showing statistically significantly greater extension than F5, as shown in FIG. 17.

The results of additional testing of dry films prepared with F2, F3, and F4 formulations using Instron 5544, with a 10 N load and extension rate of 10 mm/min, are presented in Table 5 below.

TABLE 5
Dry testing of films.

| Formulation | UTS (MPa) | Std dev | YM (MPa) | Std dev |
|---|---|---|---|---|
| F2 | 0.304 | 0.061 | 1.695 | 0.504 |
| F3 | 0.508 | 0.192 | 2.306 | 0.900 |
| F4 | 0.509 | 0.136 | 2.565 | 0.918 |

As can be seen from Table 5, comparable results with earlier testing were observed, showing that films could be prepared with excellent reproducibility of mechanical characteristics.

### DISCUSSION

The mechanical properties of the thin films formed from the four formulations tested support their suitability as carrier materials. Even with systematic underrepresentation due to the premature failure of the samples at the Instron holding clamps, each of the films tested exceed by several orders of magnitude the reported upper tensile strength of amnion, which is 18.4±8.23 Pa when preserved by air drying and 9.9±4.14 Pa when preserved in glycerol (von Versen-Hoeynck et al., 2008). However, these values reported in the literature were hydrated tensile tests, whereas the samples tested in this example were dry tested.

F2 film had the lowest UTS strength score of 0.363±0.0213 MPa. Nevertheless, this represents a 45500-fold difference on the strength of glycerol preserved amnion. The Young's modulus of term amnion is 2.29±0.7 MPa, and preterm 3.60±1.4 MPa (Benson-Martin et al., 2006). F2 had a Young's modulus of 1.65±0.663 MPa.

The Young's modulus represents a measure of the stiffness of a material, with a greater score corresponding to a greater stiffness. Conversely, a lower score shows a material has a greater elasticity—the ability to undergo elastic deformation and return to its original shape after the deforming force has been removed. F2 is therefore both stronger and more elastic than human amniotic membrane.

The upper tensile strength of F3-F5 was greater than F2 at 0.673±0.0272 MPa, 0.644±0.490 MPa and 0.626±0.108 MPa, respectively. As discussed above, these results are an underrepresentation of the true strength of these materials due to the samples failing at the clamps, indicating that the clamping procedure had weakened the material during testing.

The least elastic of these formulations was F5, with a Young's modulus of 4.46±0.455 MPa. This modulus is within the standard deviation of the reported preterm amnion elasticity (3.60±1.4 MPa (Benson-Martin et al., 2006)), which suggests all of the crystallin-containing formulations are at least equivalently elastic or more elastic than the current gold standard carrier. It will be appreciated that the ability to conform to the shape of a patient's eye without applying physical stress to the diseased tissue is vitally important for a use as a limbal stem cell carrier.

It will further be appreciated that many applications, including surgical applications, for which a composition as herein described may desirably be used require a certain degree of dimensional stability. The swelling behaviour of crystallin hydrogels prepared via UV curing was assessed. FIG. 18 shows the percentage swelling of control (PEGDA only), and of 6% and 12% crystallin based hydrogels after 24 hours swelling in PBS, pH 7.4. As can clearly be seen, reduced swelling compared to PEGDA controls was observed for both the 6%, and the 12% crystallin hydrogels, indicating improved swelling characteristics.

### Example 6: Characterisation of Biocompatibility of Compositions Comprising Crystallin Proteins

This example describes an assessment of the biocompatibility of thin film compositions comprising crystallin proteins.

### Materials and Methods

Cell Culture

Culturing of samples and live cell imaging on the Nikon Biostation took place at 37° C., 5% CO₂ and ambient humidity. Epithelial Cell Media (MEM, 10 mL (10%) FSC, 1 mL (1%) Anti-Anti per 100 mL), and Explant Media (DMEM/F12, 10 mL (10%) FCS, 50 μL ITS, 100 μL EGF, 1 mL (1%) Anti-Anti per 100 mL) were used. Depending on the growth rate of the cells, times between media changes varied. Generally, 50% media changes were made every 3-4 days. When the cell flasks approached 80% confluency they were passaged, with excess cells used for film culture or discarded.

Gibco™ TrypLE™ Express Enzyme (1X) No Phenol Red (Gibco, 12604-013) and appropriate media were warmed in a water bath to 37° C. Media from the flasks was poured to waste and a small volume of warmed PBS added into flasks to dilute any remaining serum esterase activity. Following rinsing PBS was also poured to waste. Sufficient TrypLE™ was added to completely cover the bottom of the flask, which was then incubated while shaking for 10-15 minutes at 37° C. Once the incubation time had elapsed, cell adherence was observed. If large amounts of cells were still attached, the flask content was collected, and the TrypLE™ treatment repeated. Once all cells were detached and collected, the suspensions were gently pelleted for 7 minutes at 380 xg. The supernatant was then discarded, and the cells resuspended gently in a volume of media appropriate for the expected number of cells (1-3 mL for a confluent T75).

For cell counting, 10 μL of cell suspension was added to 10 μL of Trypan Blue (Sigma, T6146). 10 μL of the mixture was pipetted onto a counting grid and viewed under 10× magnification. After counting 3 grid areas diagonally, the following equation was performed to obtain the total number of cells:

(Number of cells counted/Number of areas counted)×2(dilution factor)×10⁴(cellular magnitude)=cells/mL

cells/mL×mL of cell suspension=Cell total

An appropriate volume of suspension was then seeded into the flask to provide a number of cells equal to the seeding density required for the flask size. 1×10⁴ cells per film were seeded for the epithelial cell film seedings.

Immunohistochemistry

Samples were cultured at 37° C. and 5% CO₂ for 7, 14 or 28 days before staining. The films were removed from their initial culture wells to new sterile plate wells for treatment. If the film was detached from the casting coverslip, it was moved separately. A new coverslip was used later when forming slides to reduce the incidence of visualising cells adhered to the glass and not to the film. The samples were washed for 5×5 min in PBS to remove the media, then fixed with 4% Paraformaldehyde (PFA) for 20 minutes. PFA was removed and samples washed for 3×10 mins in PBS.

Following fixation the samples were permeabilised in methanol for 10 minutes at −20° C., then again washed for 3×10 mins in PBS. Serum blocking was undertaken by incubating the samples for 2 hrs on a shaker in 100 mM Glycine, 0.1% Triton X-100 & 10% normal goat serum in PBS. Wash 3×10 mins in PBS.

Primary antibody was prepared in PBS-B (PBS+3% BSA)+0.5% Triton X-100, and samples were incubated with their appropriate antibody at 4° C. overnight. Unconjugated antibodies were removed by washing for 3×10 mins in PBS-B. Secondary antibody incubation was carried out in PBS-B at room temperature for 3 hrs. Washed for 3×10 mins in PBS.

Nuclear staining was achieved via 4,6-diamidino-2-phenylindole (DAPI) treatment in PBS for 60 minutes in the dark on a shaker. 5×PBS rinses were undertaken to remove any DAPI which remained, before the films were mounted onto slides in Citiflour (Electron Microscopy Sciences, 1797025) (new coverslips were placed for unattached films) and sealed with nail varnish.

Cell line cultures on film were made onto SuperFrost Plus Microscope Slides (LabServ, LBS4951+) and explant cultures on film or amnion made onto Single Concave Microscope Slides (Sail Brand, 7103).

As the experiments described in this example involved immunohistochemistry and would require the production of samples mounted onto microscope slides, trial thin-film castings of the thin film formulations were made directly onto round glass coverslips (Knittel Glass). The volumes tested were 10 μL, 50 μL, 100 μL and 150 μL. It was found that 100 μL and 150 μL volumes produced thicker films that cracked at the edges during the drying process, and 10 μL formed a film which was barely perceptible. 50 μL castings had limited deformation at the edges and visibly formed a complete film, and so was chosen for future castings.

Initial testing of the crystallin protein film's suitability as a cell carrier needed to investigate whether they could maintain and expand a cell population long term. In therapeutic treatments of limbal stem cell deficiency (LSCD), cells are cultured for 3 weeks on the amniotic membrane before surgical transplantation to the ocular surface. The minimum contact time in which successful cell transfer has been reported with clinical treatments was 3 days. However, a contact time of greater than 1 week is preferred for optimal cell transference to the patient's eye. Therefore, F2 films were seeded with 1×10⁴ cells in 50 μL of epithelial media from two in-house human corneal scrape cell lines (herein referred to as Cell Line 1 and Cell Line 2) and cultured for 28 days, and the survival and expansion of these cells assessed.

Cell adherence and outgrowth was observed by light microscopy at days 7, 14, and 28 days. Cells were fixed and stained with anti-alpha tubulin, anti-cytokeratin 3/12, or anti-smooth muscle actin, with an Alexa-Fluor 488 conjugated secondary.

Gene expression of reference genes in the thin film cultures and in control cultures was assessed by RNA expression.

### Results

The F2 thin film formulations prepared as described above were highly biocompatible. FIG. 6 shows the cellular adhesion and outgrowth on the F2 film formulation at 7, 14 and 28 days post cell seeding, visualised with anti-alpha tubulin and DAPI nuclear stain.

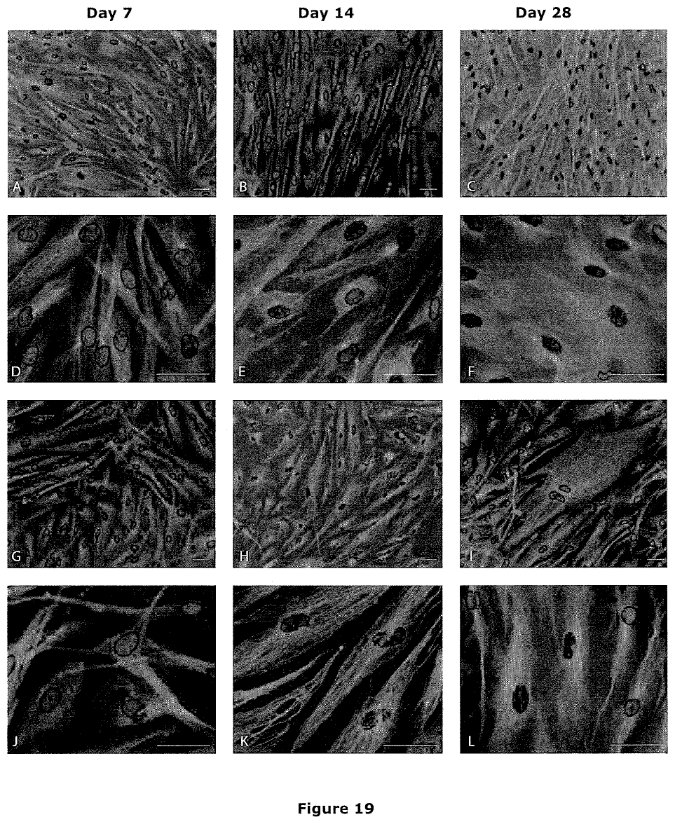

F2 thin films had excellent initial cell adhesion, as can be seen from the substantial cell density observed at 7 days (FIG. 19: A, D, G, J). Observation of cell outgrowth during culture (data not shown) found cell confluency of the entire film from the centre (seeding location) to edge within 14 days. As can be seen in FIG. 19C, by the 28th day of culture cell the cells were over-confluent and had begun to grow over each other. Comparable results were observed in replicate cultures stained with anti-Smooth muscle Actin and DAPI nuclear stain (data not shown). Notably, no overt changes in cellular morphology between the day 7 and day 28 of culture was observed, suggesting thin film formulations as described herein were not driving the cells down a different cell fate than the apparent stromal one they began culture with.

Indeed, the stromal phenotype was supported by RNA expression analysis of the cultured cells, in which no expression of corneal epithelial genes KRT12 and KRT3 was observed (data not shown). Expression of COL4A5 confirmed an ocular origin, and the lack of observed KRT13 expression indicated no conjunctival contamination (data not shown). ACTA2 and VIM expression remained nearly constant throughout the time course, and of similar fold difference to the control cells grown on tissue culture plastic (data not shown). Expression of PCNA was greater on films at all timepoints than the tissue culture plastic controls. Although there was no detection of ABCG2, TP63 and ΔNP63 on the film formulations for the initial day 7 controls, day 28 controls demonstrated weak expression.

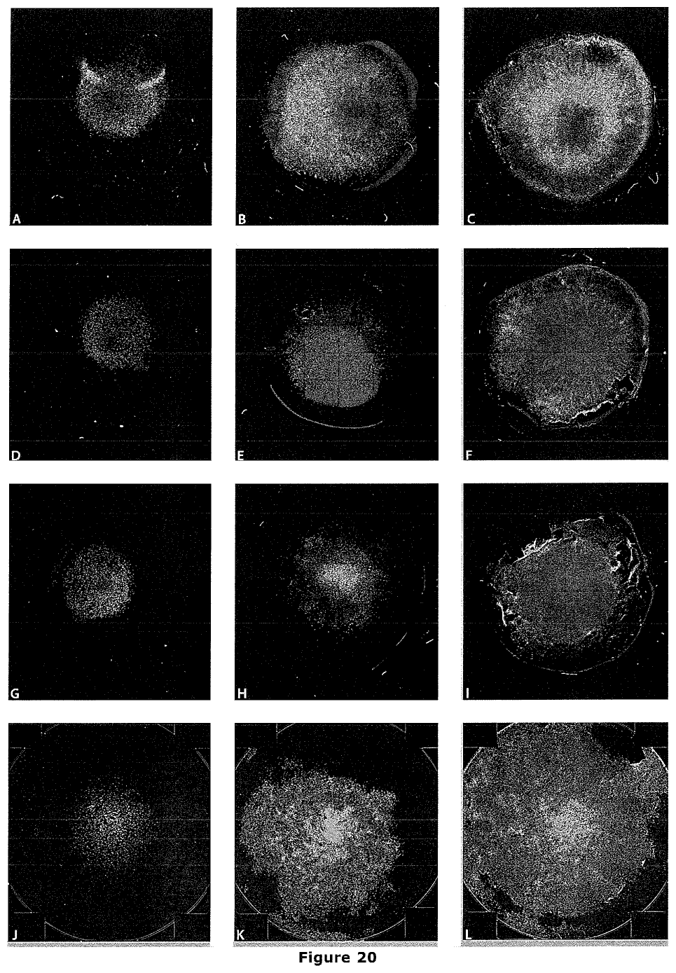

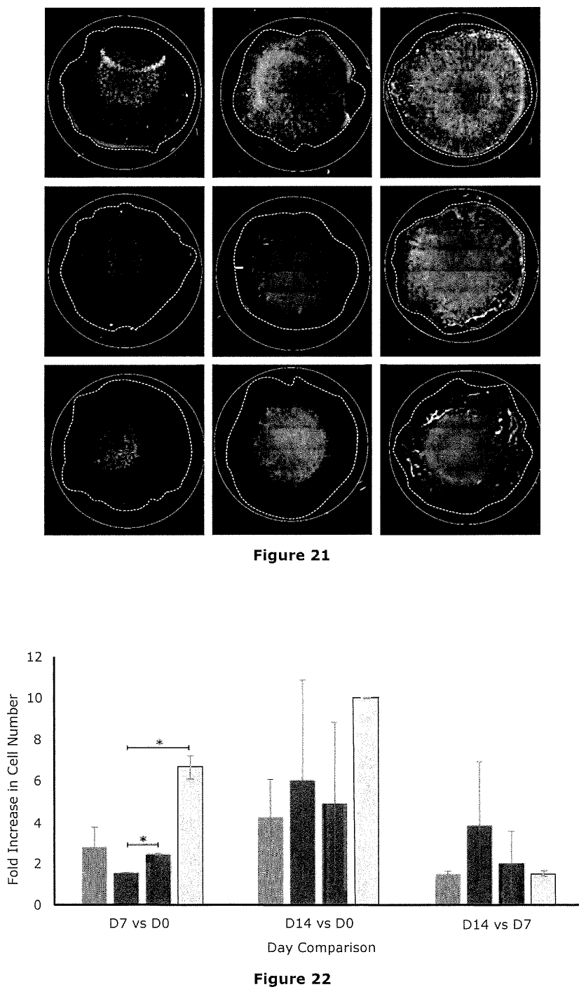

High biocompatibility was also observed with F3 and F4 thin film formulations, where DAPI staining of human corneal epithelial cells showed comparable growth to that observed for F2 formulations, as shown in FIG. 20 and FIG. 21.

Indeed, the biocompatibility of crystallin film compositions prepared as described herein was such that cell numbers in culture increased rapidly. As shown in FIG. 22, crystallin thin film formulations supported multiple fold increases in cell numbers in both the first week of culture (FIG. 22, left graph), and the second week of culture (FIG. 21, right graph), comparable overall to tissue culture plate only controls (FIG. 21, middle graph).

Overall, thin films comprising crystallin supported a proliferative cell population, and provided a growth surface which maintained equivalent fate to tissue culture plastic conditions.

### Example 7: Characterisation of Biocompatibility of Compositions Comprising Crystallin Proteins

This example describes an assessment of the biocompatibility of further thin film compositions comprising crystallin proteins.

### Materials and Methods

Thin films formed from compositions F1-F6 (see Table 3 above), each of which comprised 60 mg/mL crystallin proteins and 2% w/v glycerol, but differed in crosslinker, plasticizer, or co-initiator, as shown.

All trial films were seeded with 1×10⁴ cells in 50 μL of media, and given 10 minutes for the cells to settle and adhere before being overlaid with additional media. That same day, light microscopy was undertaken to observe the level of initial cell adherence to the films within that 10-minute time period.

### Results

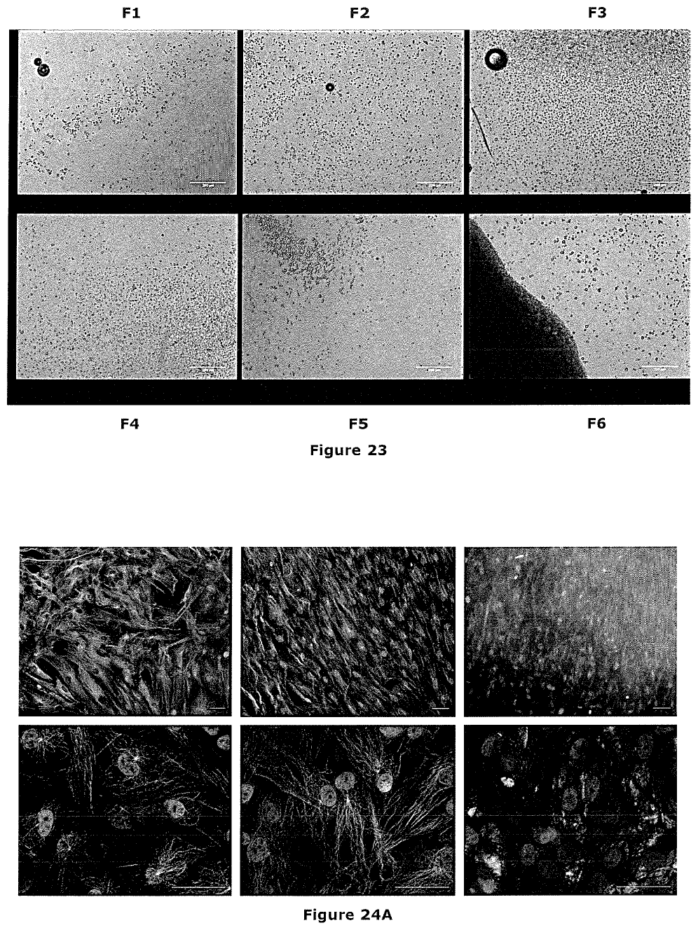

As can be seen in FIG. 23, the level of cell adhesion between the films varied significantly. F3, the R5P+L-arginine film composition (FIG. 23, top right) appeared most promising, followed by F4 (FIG. 23, bottom left) and F2 (FIG. 23, top middle). The PEGDE films, F1 (FIG. 23, top left) and F5 (FIG. 23, bottom middle), had greatly reduced adherence levels compared to F2-F4. The addition of the RGD motif in F5 did not significantly increase the level of adherence over the original F1 formulation. F6 was opaque, and discounted from further consideration (FIG. 23, bottom right).

4 days after seeding, the efficacy of the thin film formulations as a carrier was assessed using microscopy combined with a LIVE/DEAD stain (data not shown). From visual inspection, F2 and F3 had the largest and equivalent cell retention. F1, F4, and F5 had few cells. There were no dead cells present on any of the formulations. F6 remained opaque and as such no visualisation of cells was possible.

### Example 8: Characterisation of Long-Term Biocompatibility of Compositions Comprising Crystallin Proteins

This example describes an assessment of the biocompatibility of thin film compositions comprising crystallin proteins during prolonged cell culture.

### Materials and Methods

Thin films were formed from compositions F2, F3, and F4 (see Table 3 above). Human primary corneal epithelial cells were grown on these thin films for 7, 14 and 28 days and stained with DAPI nuclear stain (blue) and total alpha tubulin (red) to allow visualisation of the cytoskeleton for morphological analysis.

### Results

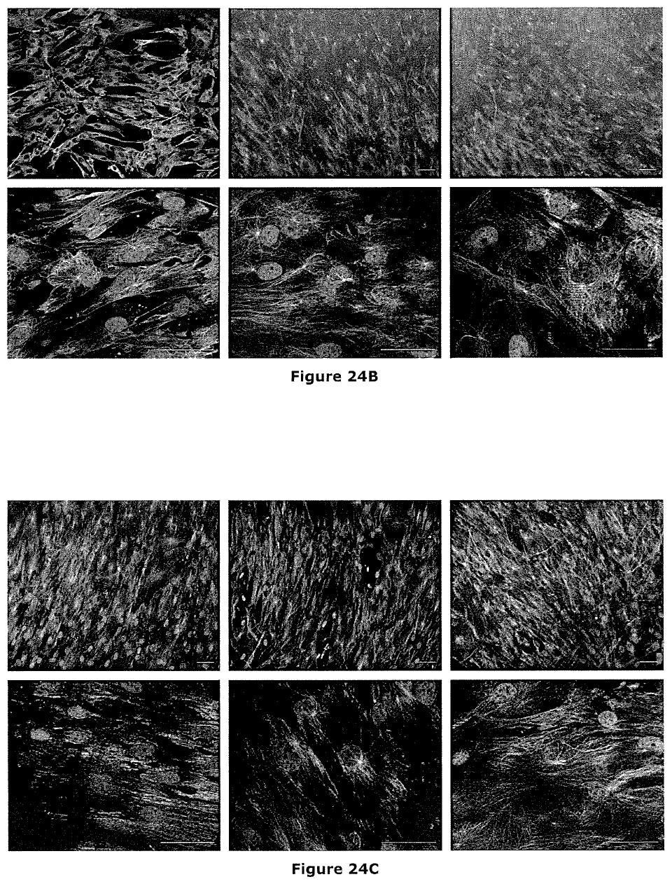

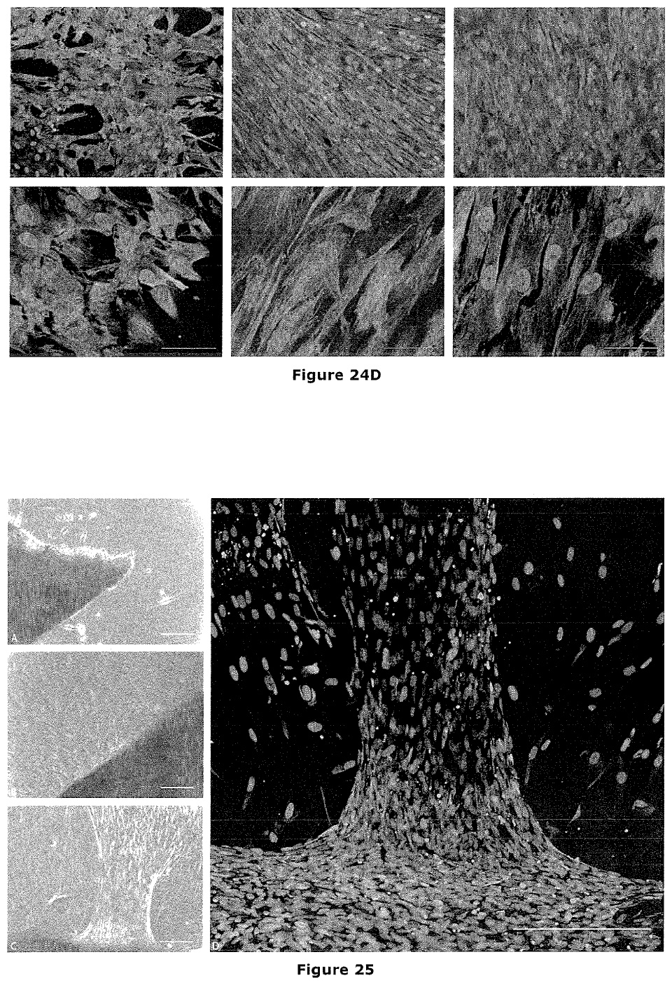

As can be seen in FIG. 24, cells grown on F2 (FIG. 24a), F3 (FIG. 24b), and F4 (FIG. 24c) showed increased alignment and density from 7 days (left), to 14 days (middle), and again at 28 days (right), over the entire period of prolonged cell culture, particularly when compared to the glass-only control (FIG. 24d). Increased alignment and density were readily apparent at both 20× (top) and 63× (bottom) magnifications.

### Example 9: Characterisation of Biocompatibility of Compositions Comprising Crystallin Proteins

This example describes an assessment of the biocompatibility of thin film compositions comprising crystallin proteins.

### Materials and Methods

Corneal Limbal Explants

Limbal explants were harvested within a UV treated Class 2 hood. Briefly, a piece of sterile gauze was pinned to a cork board that had been soaked in ethanol. Using sterilized forceps, the donor corneal rim was removed from the transport media and placed onto the sterile gauze. The rim was then pinned to the gauze covered board using ethanol sterilized pins, positioned in such a way as to apply tension to stretch and flatten the tissue. Using a scalpel, an incision ⅓ the depth of the anterior limbal surface was made, and excess cornea and sclera tissue was removed. The anterior limbal surface was then filleted away and placed into a small volume of transport media in a petri dish lid before being sectioned into 1 mm wide pieces. The explants were then placed onto their culture surfaces, allowed to settle and adhere for 10 minutes, before the careful addition of media from the sides.

Amnion Disks

Human amnion was acquired from the New Zealand National Eye Bank, and stored in glycerol on nitrocellulose filter paper at −20° C. For processing for tissue culture, the surface of the amnion was gently scraped with a cell scraper following incubation in TryPLE Express for 10 minutes, and the surface rinsed with sterile PBS. An 8 mm sterile biopsy punch (Miltex, REF33-37) was then used to obtain a section for culture.

Human corneal explant outgrowth experiments were performed on the four representative thin film formulations F2-F5. Explant outgrowth experiments with 5 individual donors, and RNA expression analysis with a further 2 donors, were performed.

For the explant experiments, 1 mm pieces of the anterior ⅓ of the limbus were dissected and placed upon the crystallin-containing thin film formulations, with amnion as a control, gold standard surface. Triplicate explants were conducted per film formulation, with duplicates for amnion.

Due to the vast number of explants outgrowths produced, a representative replicate of each biological replicate was stained and imaged, however live cell light microscopy of all replicates was taken at 4 time points.

### Results

There was a strong cellular outgrowth from all donor explants onto the F2 thin film formulations. The first cells were visible migrating from the tissue onto the surface at 4 days of culture. The explants had successful adhered to the crystallin films, and over the course of the 14 day incubation all three donor explants had fully populated the film surfaces (Average casting diameter ˜12 mm).

Representative light and fluorescent microscopy visualisations of limbal explants on F2 thin films are shown in FIG. 25. The composition of the outgrowth was variable with both time of incubation and donor. Outgrowths from donors 1 and 2 were stromal in appearance, being elongated and highly migratory (see for example FIG. 25A, 25B), and staining positive for vimentin (Red) (FIG. 25D). In comparison, donor 3 first expanded an epithelial population, demonstrated by the presence of distinct cobble stone morphology of cells and by the lack of vimentin staining (data not shown).

Associated with the stromal outgrowth in donors 1 and 2 were the formation of the distinct, elevated tissue bridges that grew out from the limbus. As shown clearly in FIG. 25C, these bridges are of a stromal composition and resemble anchor cables, firmly adhering the explant to the surface of the F2 films.

Explant outgrowth on F3 films were comparable to those on F2 films. The tissue adhered to the film surface, and the first cell outgrowths were again seen at 4 days (data not shown). Donor 1 exhibited the formation of the cellular outgrowth bridges, but also expanded a larger proportion of epithelial cells, having a less ubiquitous vimentin expression (data not shown). Donor 2 had a similar morphology, with the addition of raised ridges of cell separate from the explant. Similar to the outgrowth pattern on F2, donor 3 again had a preliminary outgrowth of epithelial cells in a tightly packed array (data not shown).

Following some physical disruption, donor 1 explant on F4 thin film needed to be repositioned, after which rapid migration of cells onto the film surface from the explant took place between days 4 and 7. The bridge formation characteristic of donor 1 was observed by 11 days of culture, and surrounded the explant by 14 days (data not shown).

Explants on F5 thin films supported the growth of several small, isolated cell populations, but limited cellular outgrowth. Notably, stem cell migration and expansion were observed with donor 2 explants, with multiple clusters of ABCB5 labelling visible on the film at 14 days and the establishment of a proliferative cell population was observed (data not shown). Stem cell spheres were seen forming at day 11 of culture, and a structure similar to the anchoring tissue bridges seen on all other donor 2 outgrowths was seen (data not shown). The explant for donor 3 successfully adhered to the F5 film, although cellular outgrowth was not seen until day 7 (data not shown), somewhat later than cell migration observed on formulations F2-F4, where cells were migrating onto the surface by 4 days.

Notably, successful visualisation of ABCB5 labelling was seen in at least one donor outgrowth for each film population, indicating the presence of viable stem cell populations.

Amnion Controls

In contrast to the thin film formulations comprising crystallin, the outgrowth of explants on amnion membrane was variable. Donor 1 explants produced a large and confluent cell outgrowth from the explant, staining positive for vimentin (data not shown). Donor 2 had a small, asymmetrical outgrowth, extending to the upper and left side of the tissue, but only a small edge present on the lower side. The explant from donor 3, although adhered to the membrane, did not produce any cellular outgrowth onto the amnion during the 14 day culture period.

Visualisation of the outgrowths on amnion was made difficult by the presence of the stabilising nitrocellulose filter, which was opaque and made high magnification imaging non-viable. At the 5× magnification, a small population of ABCB5 positive cells was observed just above the upper edge of the donor 2 explant.

RNA Expression

The gene expression data from the film explant outgrowths was compared to explant outgrowth expression on amnion controls. In all instances, bar PCNA expression on F2, the assessed film formulations had a several fold increase in expression of proliferation markers PCNA and MKI67 compared to amnion. Both epithelial and stromal markers were expressed on all films, indicating a successful outgrowth of all cell types present in the native limbal niche. There was a notable increase in expression of VIM and KERA compared to amnion, which suggests that crystallin-containing films promoted the expansion of corneal stromal cells. The observed decrease in the expression of TP63, ΔNp63 and Notch1 was expected in a rapidly expanding population.

Table 6 presents the number of copies per μl of the stem cell markers ABCB5 and ABCG2 detected in samples at 14 days averaged between explant donors 6 and 7 when grown on thin film formulations F2, F3, F4 and F5, and on amnion.

TABLE 6
Stem cell marker expression - copies per μL

| | Formulation | | | | |
|---|---|---|---|---|---|
| Marker | F2 | F3 | F4 | F5 | Amnion |
| ABCB5 | 0.22 (±0.15)* | 0.21 (±0.21)* | 0.00 (±0.00)* | 0.17 (±0.09)* | 0.00 (±0.00)* |
| ABCG2 | 1.21 (±0.02)* | 0.65 (±0.50)* | 1.59 (±0.87)* | 6.66 (±2.11)* | 0.37 (±0.37)* |

*S.d

As can be seen in Table 6, expression of ABCB5 and ABCG2 was greater on all crystallin-containing films in comparison to amnion. This suggests that in addition to limbal stem cell expansion, the daughter progenitor cells were also differentiating better compared to those on amnion.

### Example 10: Optical Characterisation of Thin Film Compositions Comprising Crystallin Proteins

This example describes an assessment of the optical transparency and transmissivity of thin film compositions comprising crystallin proteins.

Thin films were prepared from formulations F2, F3, and F4 as set out in Table 3 above.

Transmittance of light across the visible spectrum (400-700 nm) was assessed for all samples.

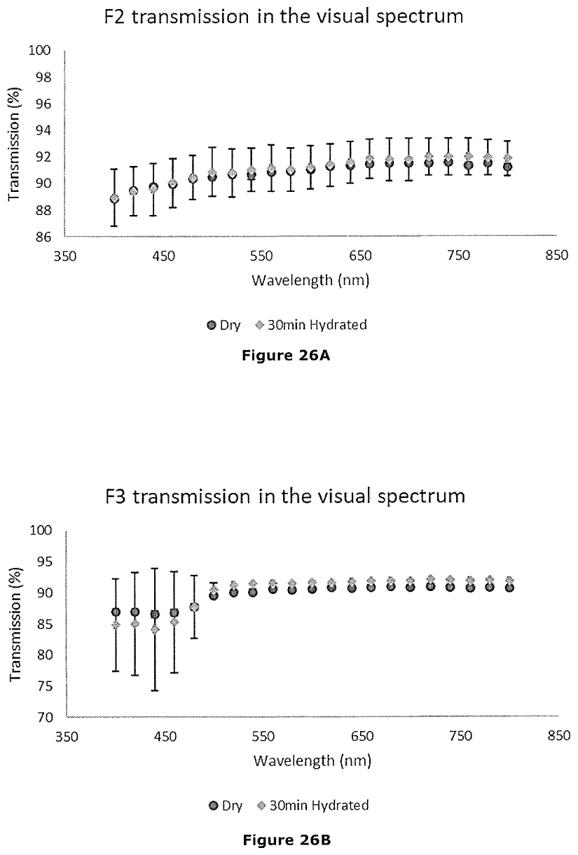

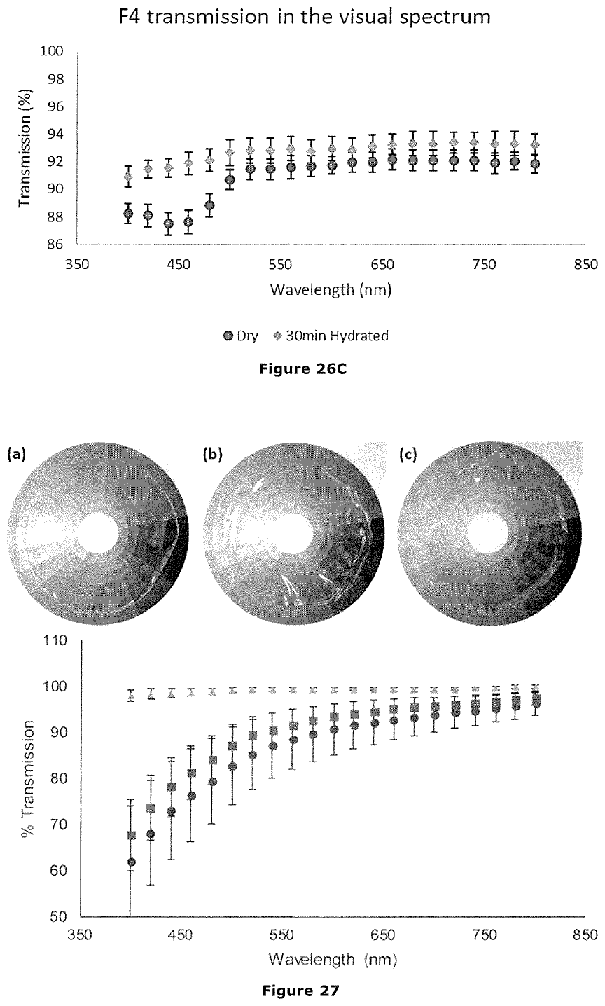

As can clearly be seen in FIGS. 26 and 27, thin films from F2, F3, and F4 have very high transmissivity over the visible spectrum in both wet and dry states. Indeed, transmissivity at or exceeding 90% across higher wavelengths was observed for each formulation, as shown in FIG. 26A for hydrated and dry F2 formulations, in FIG. 26B for hydrated and dry F3 formulations, and FIG. 26C for hydrated and dry F4 formulations. Hydrated F2, F3, and F4 formulations showed excellent colour uniformity (see FIGS. 27a, 27b, and 27c, respectively), and additional transmission assays again showed high transmittance, in which all films exhibited better that the 72% transparency threshold required for ocular applications (see FIG. 27, bottom).

### Example 11: Preparation of Compositions Comprising PEGylated Crystallin Proteins

This example describes the preparation of compositions comprising PEGylated crystallin proteins.

PEGylation strategies were explored to assess any impact on the stability of crystallin proteins, for example, for both improved shelf-life and efficacy, and to obtain crystallin hydrogel via cohesive cross-linking of crystallin proteins. Following extraction, the crude crystallin extract was reacted with different PEG derivatives to optimise access to functional groups for cross-linking to aid gel formation.

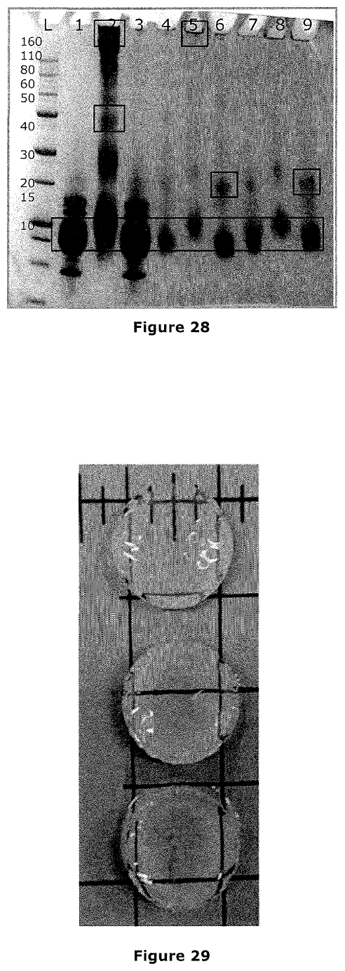

The PEGylation reaction was assessed by using SDS-PAGE to characterize PEG-crystallin conjugates. The presence of high molecular weight species on SDS-PAGE in the presence of PEG, as shown in FIG. 28, confirmed successful crystallin PEGylation.

### Example 12: Optical Characterisation of Adhesive Compositions Comprising Crystallin Proteins

This example describes an assessment of the optical transparency and transmissivity of adhesive compositions comprising crystallin proteins.

Different concentrations of PEGDA, crystallin and photoinitiator (both riboflavin and irgacure 2959) were initially screened to obtain a PEGDA based crystallin hydrogels.

Representative embodiments were prepared using a concentration of PEGDA and irgacure 2959 at 15 and 0.5 (w/v %), respectively, with a maximum concentration of crystallin proteins in the hydrogels of 120 mg/mL to obtain transparent hydrogels. Initial visual screening of PEGDA-only and PEGDA+crystallin protein hydrogels showed that the inclusion of crystallin at 60 mg/mL and at 120 mg/mL did not negatively affect hydrogel transparency, as seen in FIG. 29 middle and FIG. 29 bottom compared to FIG. 29 top.

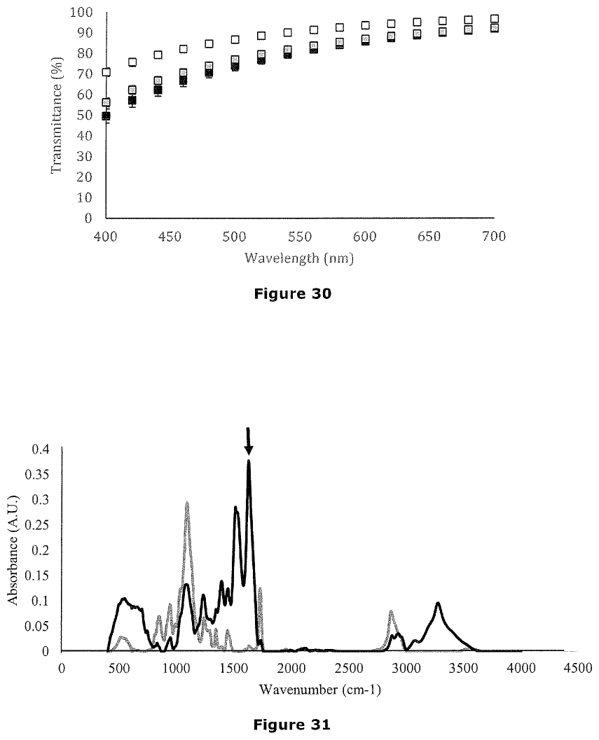

Transmittance of light across the visible spectrum (400-700 nm) was assessed for both the pre-cured samples, and cured hydrogels. Hydrogels were cast using different volumes of pre-cured premix samples (ranging from 50 to 600 uL). Both pre-cured samples and hydrogels showed a high transmittance (>80%). The current suggested threshold suitable for materials for human corneal transplantation is 72% (Gonzalez-Andrades et al. 2015). The optical transparency of the hydrogels (casted using 600 uL of the pre-polymer solution) is shown in FIG. 30.

As can clearly be seen in FIG. 30, hydrogels comprising crystallin protein-containing hydrogels with extremely high optical transparency and transmissivity over the visible spectrum can be obtained using the compositions described herein.

To confirm the incorporation of crystallin proteins in PEGDA hydrogels, ATR-FTIR was done. To remove any un-crosslinked crystallin, hydrogels were placed in water for 24 hrs, followed by overnight drying at 37° C., before FTIR analyses. A representative FTIR graph comparing PEGDA only, and PEGDA-crystallin hydrogels is shown in FIG. 31. The peaks at 1627, 1637, 3300, 3100, 619 nm correspond to beta-sheets, NH stretching, Ist amide, and OCN bending, respectively.

The peaks at 1627 nm in PEGDA-crystallin sample confirm the presence of crystallin proteins, as crystallin proteins are known to exhibit beta-sheet structure. Indeed, the FTIR spectrogram for PEGDA-crystallin is highly comparable to that observed with the crude crystallin extract as described in Example 2 above and shown in FIG. 10.

### Example 13: Physical Characterisation of Compositions Comprising Crystallin Proteins

This example describes the assessment of certain physical characteristics of representative compositions comprising crystallin proteins.

Crystallin compositions F2, F3, and F4 were prepared as described above.

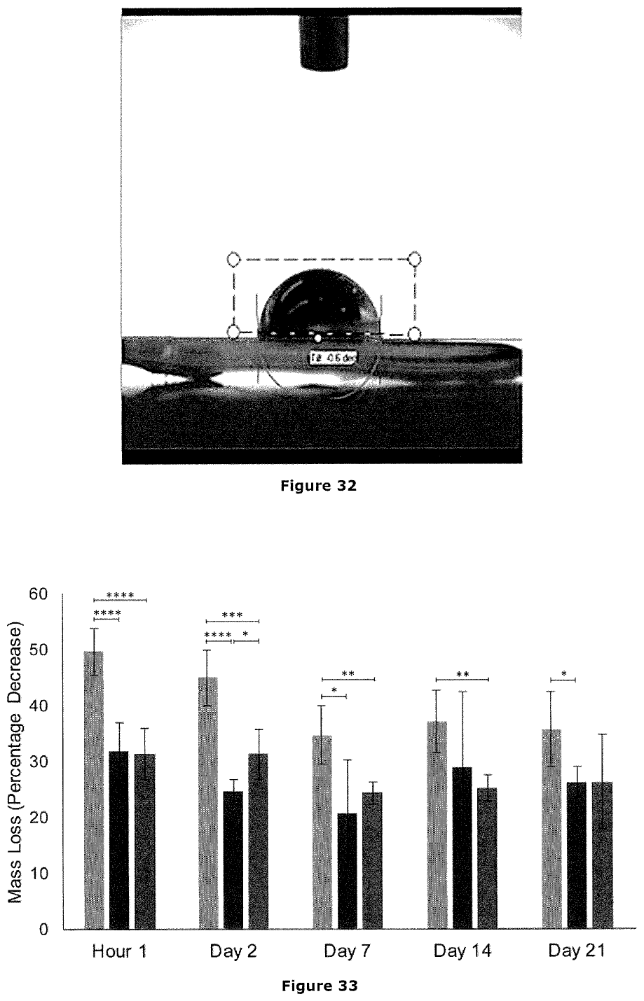

Contact angle values were measured as depicted in FIG. 32 to determine the wettability of these crystallin films. As can be seen in Table 7 below, the contact angle value of each crystallin protein formulation was below 90°, classifying these compositions as hydrophilic materials.

TABLE 7
Contact angle values for crystallin compositions

| Formulation | Average Angle |
|---|---|
| F2 | 64.8 ± 12.7 |
| F3 | 75.8 ± 7.4 |
| F4 | 73.8 ± 15.2 |

The stability of these crystallin films was then assessed by measuring mass change during hydration over a 21 day period. As can be seen in FIG. 33, the majority of mass lost (compared to starting mass) from films upon hydration occurred within the first hour following hydration. This rapid mass loss is the result of a rapid diffusion of unbound protein. After this initial loss, the mass of the hydrated films remained stable over the following 21 days.

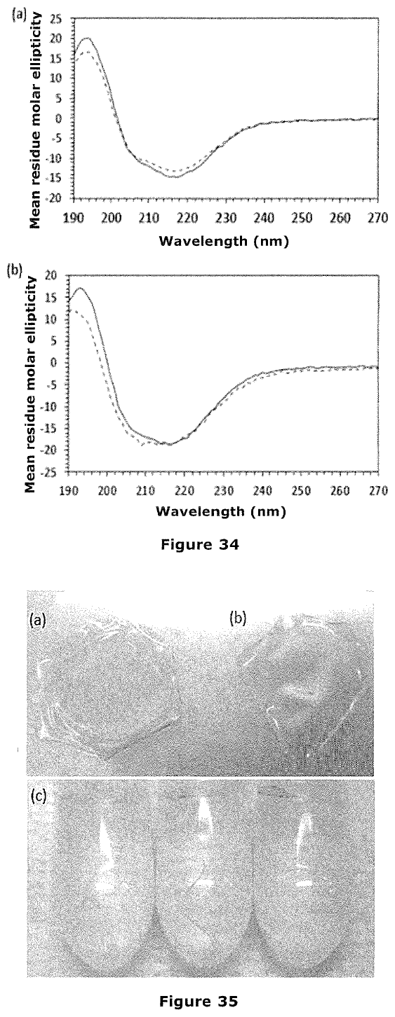

The ability of these crystallin films to undergo sterilization and retain useful structural integrity and function is of critical importance to their use in desirable applications, such as in surgical procedures. Crystallin compositions F2 and F3 were gamma sterilized at 25-32 KGy. FIG. 34 presents the results of circular dichroism spectroscopy of crystallin protein leached from F2 film (FIG. 34a) and from F3 film (FIG. 34b), where the solid line data represents films incubated in milliQ for 24 h, and the dotted line data is from gamma sterilized films incubated in milliQ for 24 h. The presence of a minimum negative peak at 217 nm, indicative of β-sheet structure in samples from gamma sterilized and from non-sterilized films confirmed that gamma sterilization had no adverse impact on the native structure of crystallin proteins in films.

FIG. 35 shows representative images of a crystallin film after storage at room temperature for 3 months (FIG. 35a), of a crystallin film after gamma sterilization as described above (FIG. 35b), and of hydrated film samples after gamma irradiation, showing that these films retained their structural integrity and remained insoluble.

### Example 14: Functional Characterisation of Adhesive Compositions Comprising Crystallin Proteins

This example describes an assessment of the adhesive efficacy of adhesive compositions comprising crystallin proteins.

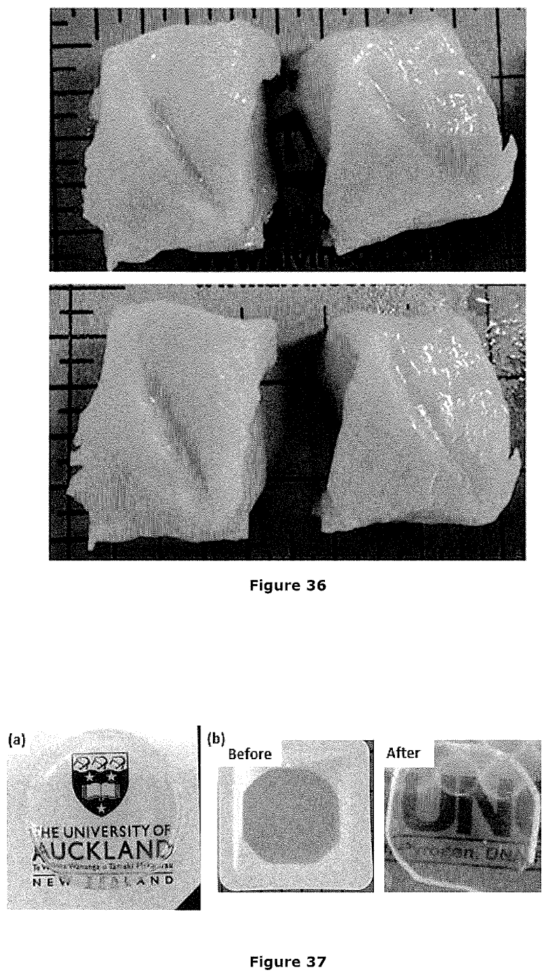

To demonstrate the adhesive properties of the compositions, chicken breast samples were used as a representative of soft wet tissue, as it is easily available and inexpensive. An incision was made on chicken breast sample using a scalpel blade and 200 μL of PEGDA only (FIG. 36, top left) and PEGDA based crystallin (FIG. 36, top right) pre-cured samples with 1% photoinitiator were applied, followed by UV exposure for 3 min.

The coated solution was immediately converted to a swollen gel and the gel tightly adhered to the tissue (FIG. 36).

As can readily be seen in FIG. 36, the crystallin-containing composition (FIG. 36, bottom right) effectively sealed the incision, remaining closed despite physical stretching sufficient to open the incision to which the PEGDA-only control (FIG. 36, bottom left) composition was applied.

These data support the adhesive efficacy of representative crystallin-containing compositions described herein. These examples clearly demonstrate that biopolymer compositions comprising one or more crystallin proteins as described herein are suitable for use as bioadhesives, and given their high transparency, are particularly suited to use in ocular surgery.

### Example 15: Functional Characterisation of Adhesive Compositions Comprising Crystallin Proteins

This example describes an assessment of the adhesive efficacy of adhesive compositions comprising crystallin proteins.

PEGDA crystallin adhesive formulations suitable for either visible light curing, or UV curing, were prepared according to Table 8 below.

TABLE 8
UV- and visible light adhesive compositions

| Method | Crosslinking agent | Polymer | Co-initiator | Crystallin |
|---|---|---|---|---|
| (a) UV curing@ | Irgacure 2959 - | PEGDA - | — | 10-120 mg/mL |
| 365 nm | 0.05-0.1% | 10, 15 and 20% | | |
| (b) Visible light @ | Eosin Y - | PEGDA - 10% | TEA* - 0.75-1.5% | 10-120 mg/mL |
| 365 nm | 0.05-0.1 mM | | NVP** - 37 nM | |

*Triethanolamine
**N-vinylpyrrolidinone

Representative images showing the visual characterisation of these compositions are presented in FIG. 37, in which the UV cured formulation is shown in FIG. 37a, and the visible light formulation before (FIG. 37b, left) and after (FIG. 37b, right) are show. The highly transparent nature of these cured formulations supports their applicability to use in ocular surgery.

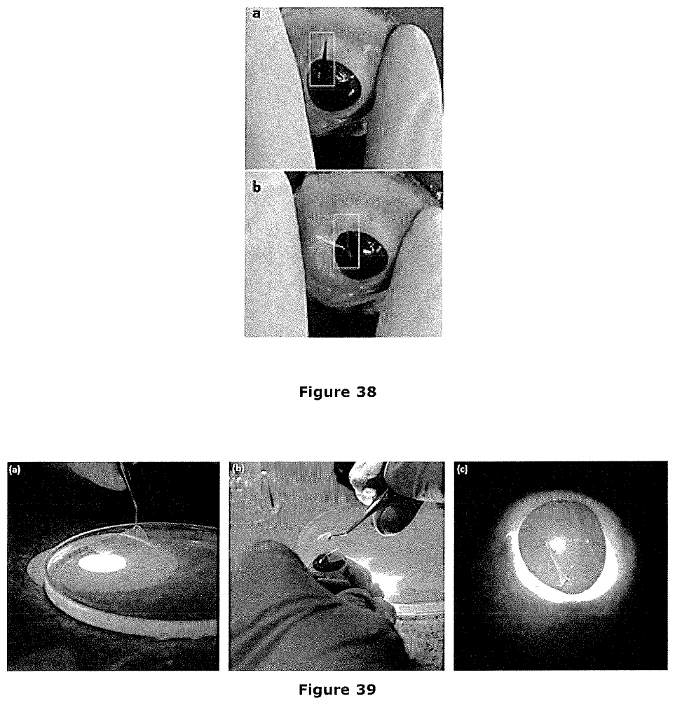

To investigate the adhesive properties of the compositions and their suitability for use in surgical applications, pig eye samples were used as a representative ocular tissue. An incision was made on the eye as shown in FIG. 38a using a scalpel blade.

Crystallin hydrogel composition was then applied to the incision, and cured for 3 minutes (see FIG. 38b, UV-cured crystallin composition).

As can readily be seen in FIG. 38(b), the crystallin-containing composition sealed the incision in wet conditions (37° C.) and remained sealed for up to 2 days. This is ample time in which to perform further surgical procedures should they be required.

Additionally, the surgical tractability of crystallin films was established in a suture test using a porcine eye model depicted in FIG. 39. As can readily be seen in FIG. 39a, crystallin film formulation F3 had good foldability when hydrated and didn't stick to itself, which allowed folds to be reversed and eased handling. FIG. 39b shows that F3 films can be easily cut, lifted, and placed onto the corneal surface, and as shown in FIG. 39c, crystallin films can be readily sutured.

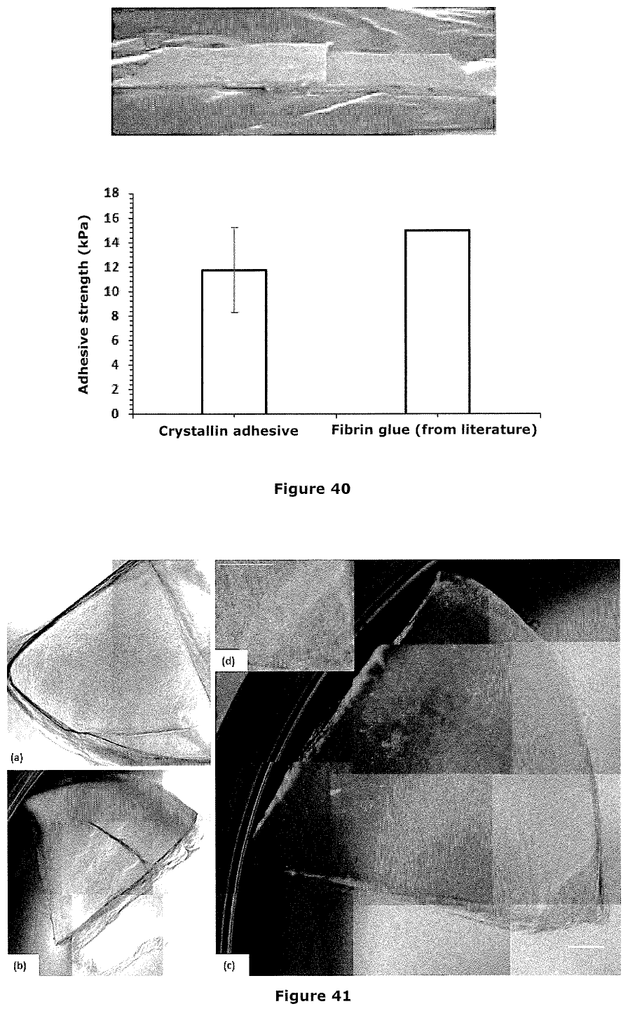

The adhesive strength of UV cured crystallin formulations was determined in a lap shear test, using porcine skin samples as shown in FIG. 40, top. FIG. 40 bottom presents data on the adhesive strength from this test, in which error bar represent the standard deviation of mean taken from six samples. The fibrin glue value is taken from literature (Nakayama & Matsuda, 1999). As can be seen, the adhesive strength of crystallin films is comparable to that reported for fibrin glue.

These data support the adhesive efficacy of representative crystallin-containing compositions described herein. These examples clearly demonstrate that biopolymer compositions comprising one or more crystallin proteins as described herein are suitable for use as bioadhesives, and given their high transparency, are particularly suited to use in ocular surgery.

### Example 16: Characterisation of Biocompatibility and Cellular Transfer Efficacy of Compositions Comprising Crystallin Proteins

This example describes an assessment of the biocompatibility of thin film compositions comprising crystallin proteins, and particularly their efficacy in transferring a cellular population.

### Materials and Methods

An 8 mm biopsy punch was used to remove the central cornea from donated human tissue. The cornea was decellularised by 3 freeze/thaw cycles in sterile MilliQ at −80° C. Following decellularisation the tissue was rinsed 3 times in sterile MilliQ to remove any loose cell debris, before being treated in 4 U/mL DNase I overnight at 37° C. DNase was inactivated using 5 mM EDTA. LIVE/DEAD staining displayed no live cells. Corneal tissue was washed 5× in sterile PBS before being sectioned into 5 equal segments and arranged as follows:

1. Above cell free control film

2. Above 18-138A culture film

3. Below 18-138A culture film

4. Above 18-147 culture film

5. Below 18-147 culture film

F2 film formulations were cast onto 13 mm glass coverslips and cured. 2×10⁴ cells from two primary human corneal epithelial cell lines at P3 and P4 were seeded onto 6 films (3 for each cell line). These were cultured for 7 days to allow the cells to proliferate across the film surface.

Samples were incubated at 37° C. for 7 days. At 7 days corneal tissue was removed from its treatment surface and LIVE/DEAD stained. Following imaging tissue was returned to culture separate from the treatment surfaces to assess further proliferation.

### Results

The transfer of human epithelial cells using F2 thin films onto decellularised human cornea was highly effective, as can readily be seen in FIG. 41, in which FIG. 41a shows a cell free control, FIG. 41b shows cornea placed on top of cultured cells, FIG. 41c shows cornea placed under cultured cells, and FIG. 41d shows cornea placed under cultured cells at ×10 magnification. Cell proliferation and attachment was readily apparent after transfer. The presence of live cells (green), the absence of dead cells (red), and the observed proliferation and attachment of cells to the cornea confirm the transfer of healthy cells to the corneal surface. These data confirm that crystallin films can be used as a successful cell carrier, for example to repopulate decellularized cornea and other target tissues.

These data established the surprising efficacy of compositions comprising crystallin proteins as herein described in cell transfer applications, including in the transfer of stem cell populations, such as corneal epithelial cell populations and/or stem cell populations for use in ocular therapies.

### Example 17: Functional Characterisation of Active Agent Delivery Compositions Comprising Crystallin Proteins

This example describes an assessment of the ability of hydrogel compositions comprising crystallin proteins to provide delivery of active agents, in this case ocular antibiotics.

### Materials and Methods

All thin films used in this example comprised crystallin—120 mg/mL, Glycerol—2%, glutaraldehyde 10 mM.

To measure the drug release rate, chloramphenicol was loaded into the crystallin films. Crystallin films were cast with the drug loaded into the solution on 10 mm PDMS sheets. These films were then placed in the oven overnight to dry. Once dry, films were placed in 1 mL Eppendorf tubes before being soaked in a 500 uL solution of either PBS or Simulated Tear Fluid (STF, comprising NaCl 0.68 g, NaHCO₃ 0.22 g, CaCl₂.2H₂O 0.008 g. KCl 0.14 g and distilled deionised water to 100 mL.). 100 uL samples were then taken at specific time slots: 5, 10, 15, 20, 25, 30, 60, 180, 300, 360 minutes and analysed on a plate reader at 271 nm and 230 nm to determine Chloramphenicol concentration. For multi-layered films (3 layers) only the middle film contained the drug (Chloramphenicol). Multi-layered films were made with 3 layers of the following composition: crystallin—120 mg/mL, glycerol—2%, GA—10 mM with the middle layer containing the chloramphenicol at the test concentration (3 mg/mL, 5 mg/mL, 10 mg/mL and 15 mg/mL). The first layer was cast on a 10 mm PDMS sheet and once dried, the second (middle) layer was cast on top, and when that was dry the final layer was cast on top.

Analysis Method

Standard curves of chloramphenicol were created using absorbance before testing with a range of concentration from Omg/mL to 3 mg/mL at 230 nm, 240 nm, 254 nm and 271 nm. These curves were then used to convert the absorbance obtained from the samples and convert to concentration.

Extended Time Drug Delivery

The films were left in the solution for up to a week to determine the concentration of Chloramphenicol after 7 days. Wavelengths 230 nm and 271 nm produced a control with very minimal noise and readings at these wavelengths were preferred for analysis.

### Results

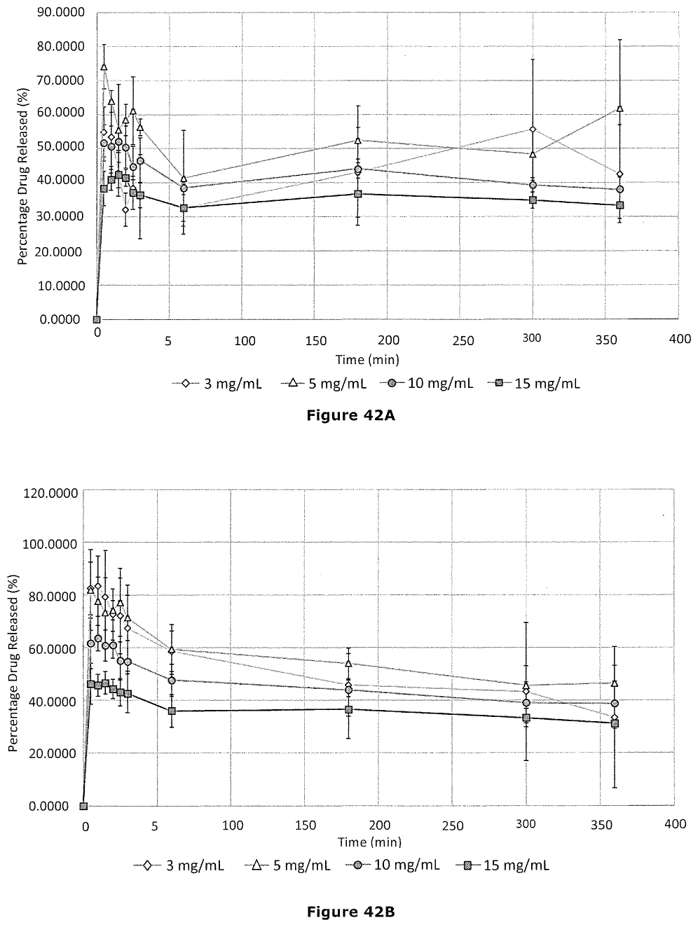

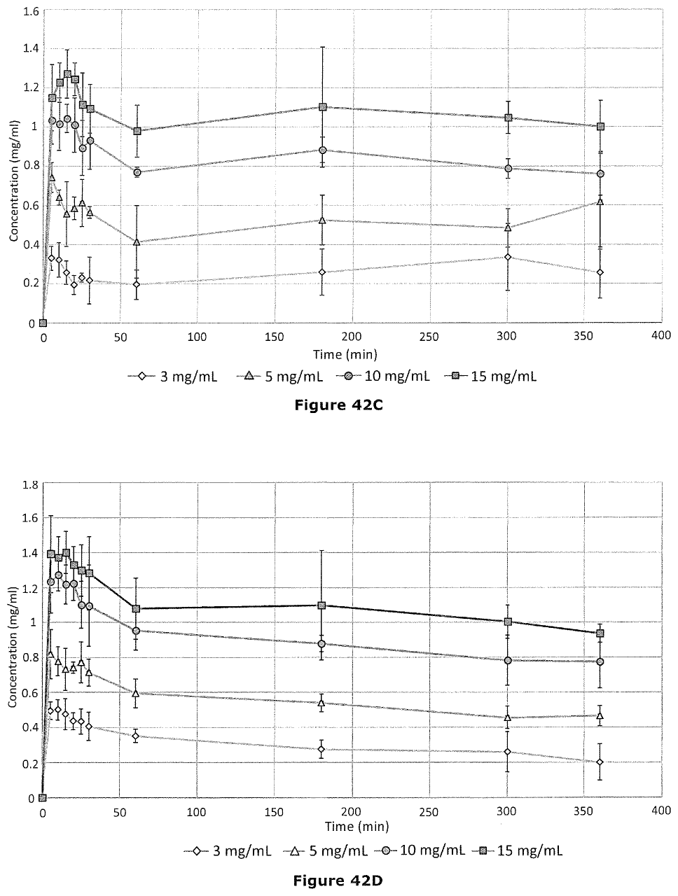

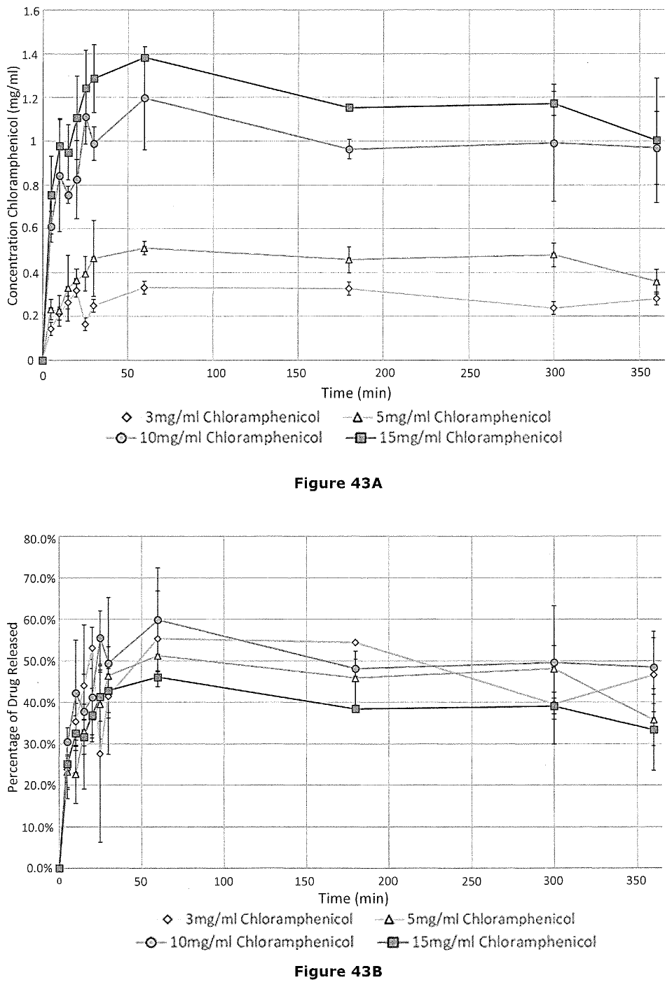

For single layer films, drug release peaked at 10-25 minutes for all drug loadings tested (see FIG. 42A, 42B). % drug released (amount of drug released into the solution as a percentage of amount of drug loaded) followed a similar profile as the concentration (see FIG. 42C, 42D). All films were able to reach at least 70% drug release—with the 10 mg/mL achieving 100%.

The readings at 230 nm and 271 nm for drug concentration were very similar with 271 nm reading slightly elevated levels of chloramphenicol for all samples at the initial peak (cf FIGS. 42C and 42D). Drug concentration in solution reflected initial drug loading, with very good agreement between both wavelengths.

Likewise, there was very good agreement between both assay wavelengths when assessing % drug delivery, with highly comparable results for each loaded concentration (cf FIGS. 42A and 42B).

The same trend was observed with all films, with a large initial increase in chloramphenicol concentration in solution reaching a plateau at around 15-25 minutes, then eventually decreasing and achieving a relatively steady state concentration past the 50 minute point.

Very similar results to the PBS trial were obtained with STF, where there is an increase in drug delivery percentage and concentration up to a peak at or before 30 minutes, with a plateau thereafter (data not shown).

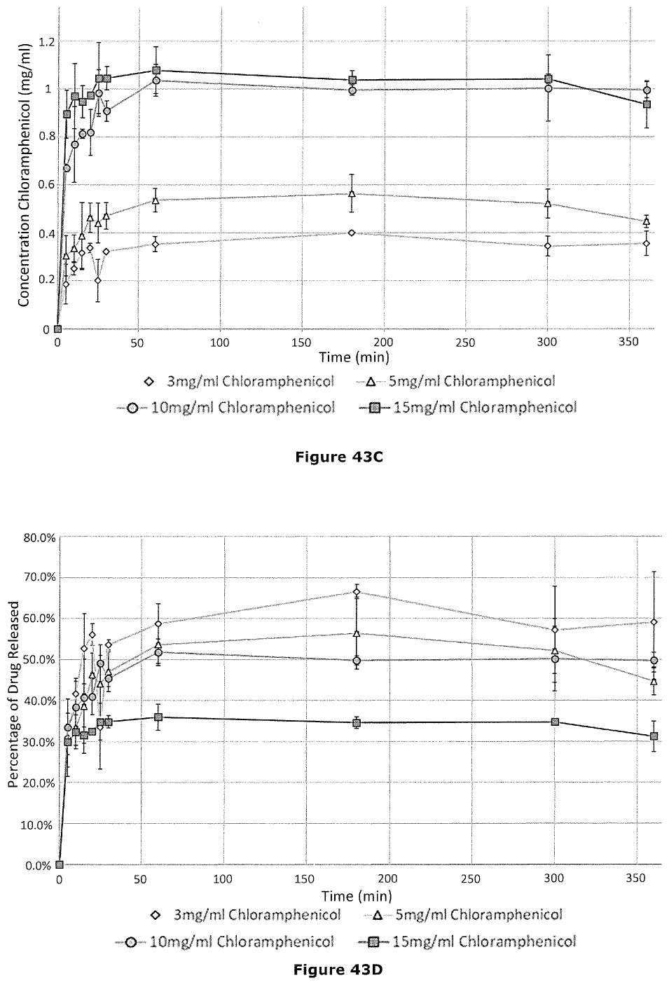

Both % drug released, and overall drug concentration in the solution, were reduced with multi-layer films compared to single layer films with the equivalent drug loading, as shown in FIG. 43B (230 nm) and FIG. 43D (271 nm), and FIG. 43A (230 nm) and FIG. 43C (271 nm), respectively. However, the release profile of these films was more stable and predictable, as shown in FIG. 43A-D. The overall trend is comparable to the single-layered films (with a plateau leading to an eventual decrease until it reaches a steady-state) but the decrease in concentration after peak is markedly reduced. The single layer films have a decrease in concentration (from maximum to the steady state region) of about 0.2-1.2 mg/ml (higher for the 10 mg/ml film) while the multi-layered films had a decrease of 0.1-0.2 mg/ml (for all films). Drug release from the multi-layer films is thus more constant, as indicated by the smoother curve.

The release plateau for the multi-layered films is seen between 60 and 180 minutes, with the steady region immediately following.

The % drug delivery is consistent with drug loading, in that the higher concentration films produced a higher concentration of drug in the solution.

### Example 18: Functional Characterisation of Active Agent Delivery Compositions Comprising Crystallin Proteins

This example describes an assessment of the ability of hydrogel compositions comprising crystallin proteins to provide delivery of an active agent, in this case the antibiotic tetracycline.

### Materials and Methods

In this experiment, tetracycline was added to UV cured crystallin hydrogel compositions (0.1% irgacure 2959, 10% PEGDA) during polymerisation (referred to here as ‘fresh gels’), and separately was absorbed into dried UV cured hydrogels of the same formulation (referred to here as ‘dried gels’). Drug release was assessed over 7 days as described above.

### Results

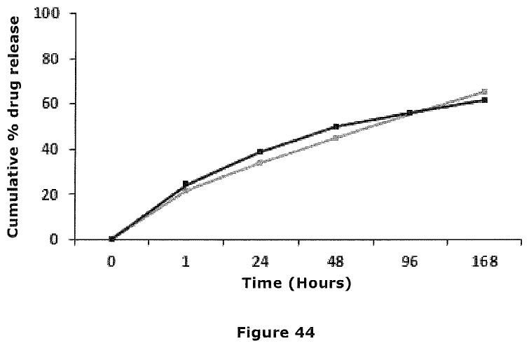

As can be seen in FIG. 44, which depicts cumulative drug release, the release profile from fresh gels (black line data) was comparable to that of dried gels (grey line data), as was total drug release overall. Hence, effective delivery of tetracycline antibiotic can be achieved irrespective of whether the tetracycline is introduced into the delivery composition during or after polymerisation. This affords desirable flexibility in preparing active agent delivery compositions for particular applications.

These examples clearly demonstrate that biopolymer compositions comprising one or more crystallin proteins as described herein are suitable for use as drug delivery materials, for example the delivery of ocularly-effective antibiotics, for example in ocular surgery or other ocular therapies.

### PUBLICATIONS

Benson-Martin, J., Zammaretti, P., Bilic, G., Schweizer, T., Portmann-Lanz, B., Burkhardt, T., . . . Ochsenbein-Kölble, N. (2006). The Young's Modulus of Fetal Preterm and Term Amniotic Membranes. European Journal of Obstetrics & Gynecology And Reproductive Biology, 128(1), 103-107.
   Doi.Org/10.1016/J.Ejogrb.2005.12.011
Gonzalez-Andrades M, Cardona JdIC, Ionescu A M, Mosse C A, Brown R A (2015) Photographic-Based Optical Evaluation of Tissues and Biomaterials Used for Corneal Surface Repair: A New Easy-Applied Method. PLoS ONE 10(11): e0142099.
   Doi.Org/10.1371/journal.pone.0142099
Horwitz, J., Huang, Q. L., Ding, L., & Bova, M. P. (1998). Lens α-crystallin: Chaperone-like properties. In Methods in enzymology (Vol. 290, pp. 365-383). Academic Press.
Mason Posner, A Comparative View of Alpha Crystallins: The contribution of comparative studies to understanding function, Integrative and Comparative Biology, Volume 43, Issue 4, August 2003, Pages 481-491, https://doi.org/10.1093/icb/43.4.481
Nakayama, Y., & Matsuda, T. (1999). Photocurable surgical tissue adhesive glues composed of photoreactive gelatin and poly (ethylene glycol) diacrylate. Journal of biomedical materials research, 48(4), 511-521.
Posner, M., Kantorow, M., & Horwitz, J. (1999). Cloning, sequencing and differential expression of αB-crystallin in the zebrafish, Danio rerio. Biochimica et Biophysica Acta (BBA)-Gene Structure and Expression, 1447(2-3), 271-277.
Runkle, S., Hill, J., Kantorow, M., Horwitz, J., & Posner, M. (2002). Sequence and spatial expression of zebrafish (Danio rerio) αA-crystallin. Molecular vision, 8, 45.
Von Versen-Hoeynck, F., Steinfeld, A. P., Becker, J., Hermel, M., Rath, W., & Hesselbarth, U. (2008). Sterilization and Preservation Influence the Biophysical Properties of Human Amnion Grafts. Biologicals, 36(4), 248-255.
   Doi.Org/10.1016/J.Biologicals.2008.02.001

The entire disclosures of all applications, patents and publications cited above and below, if any, are herein incorporated by reference.

Where in the foregoing description reference has been made to integers or components having known equivalents thereof, those integers are herein incorporated as if individually set forth.

It should be noted that various changes and modifications to the presently preferred embodiments described herein will be apparent to those skilled in the art. Such changes and modifications may be made without departing from the spirit and scope of the invention and without diminishing its attendant advantages. It is therefore intended that such changes and modifications be included within the present invention.

The invention may also be said broadly to consist in the parts, elements and features referred to or indicated in the specification of the application, individually or collectively, in any or all combinations of two or more of said parts, elements or features.

Aspects of the invention have been described by way of example only, and it should be appreciated that variations, modifications and additions may be made without departing from the scope of the invention, for example when present the invention as defined in the indicative claims. Furthermore, where known equivalents exist to specific features, such equivalents are incorporated as if specifically referred in this specification.

1. A biocompatible composition comprising:
one or more isolated, purified, recombinant, or synthesised proteins selected from the group comprising:
a. an α-crystallin;
b. a β-crystallin;
c. a γ-crystallin;
d. a protein from any one of a) to c) above from Hoki (Macruronus novaezelandiae);
e. a protein from any one of a) to c) above from Homo sapiens;
f. a protein comprising the amino acid sequence identified in Table 1 herein;
g. a polypeptide comprising or consisting of at least about 10 contiguous amino acids from any one of a) to f) above;
h. a protein having at least about 90% amino acid identity to any one of a) to g) above;
i. a protein according to any one of a) to h) above having the native structure of a crystallin protein in vivo;
j. any combination of two or more of a) to i) above;
optionally one or more plasticizers;
optionally one or more co-initiators; and
one or more crosslinkers.

2. The biocompatible composition according to claim 1, wherein said one or more proteins are crosslinkable to form a polymer.

3. The biocompatible composition according to claim 1 or claim 2, wherein the biocompatible composition is an in vivo gelling composition formulated to at least in part polymerise and/or gel at a target site in or on a subject's body, or wherein the biocompatible composition is an in vivo gelling composition formulated such that crosslinking of the in vivo gelling composition occurs or is initiated when present at a target site in or on a subject's body.

4. A method for producing a crosslinked biopolymer composition, the method comprising:
providing a composition comprising:
a. an α-crystallin;
b. a β-crystallin;
c. a γ-crystallin;
d. a protein from any one of a) to c) above from Hoki (Macruronus novaezelandiae);
e. a protein from any one of a) to c) above from Homo sapiens;
f. a protein comprising the amino acid sequence identified in Table 1 herein;
g. a polypeptide comprising or consisting of at least about 10 contiguous amino acids from any one of a) to f) above;
h. a protein having at least about 90% amino acid identity to any one of a) to g) above;
i. a protein according to any one of a) to h) above having the native structure of a crystallin protein in vivo;
j. any combination of two or more of a) to i) above;
optionally one or more plasticizers;
optionally one or more co-initiators; and
contacting said composition with one or more crosslinking molecules;
initiating crosslinking, thereby forming a crosslinked biopolymer composition.

5. A method for producing a composition comprising one or more purified crystallin proteins, the method comprising:
providing vertebrate eye tissue;
homogenising the tissue in the presence of an extraction buffer under conditions suitable for the maintenance of native crystallin protein structure;
separating the liquid homogenate from any residual solids, for example by centrifugation or filtration, to provide a crystallin protein-containing solution;
optionally at least partially further purifying the crystallin protein;
optionally dialysing the crystallin protein-containing solution to remove the extraction buffer;
optionally lyophilising the crystallin protein-containing solution to provide a lyophilised crystallin protein composition;
optionally storing the crystallin protein-containing solution or the lyophilised crystallin protein composition, for example at or below 0° C.;
wherein a substantial proportion of the purified crystallin proteins retain their native structure.

6. The method of claim 5 wherein the conditions suitable for the maintenance of native crystallin protein structure comprise
a. maintaining the homogenate in an extraction buffer at a pH of 7 or greater; or
b. maintaining the homogenate at a physiological pH; or
c. maintaining the homogenate at a temperature of below about 15° C.;
d. both a) and c) above; or
e. both b) and c) above;
and wherein the method comprises:
separating the liquid homogenate from any residual solids by centrifugation or filtration, to provide a crystallin protein-containing solution;
dialysing the crystallin protein-containing solution to remove the extraction buffer;
optionally lyophilising the crystallin protein-containing solution to provide a lyophilised crystallin protein composition;
maintaining the crystallin protein-containing solution or the lyophilised crystallin protein composition under conditions appropriate to maintenance of native crystallin protein structure, for example at or below about 4° C. until use;
wherein a substantial proportion of the purified crystallin proteins retain their native structure.

7. The method of claim 5 or claim 6, wherein the vertebrate eye tissue is lens tissue or phacoemulsification material.

8. The method of any one of claims 5 to 7, wherein the vertebrate eye tissue is eye tissue from fish or is eye tissue from a mammal.

9. The method of any one of claims 5 to 8, wherein homogenisation is performed under conditions to avoid or minimise disruption of one or more crystallin isoforms and/or to avoid or minimise protein aggregation.

10. The method of any one of claims 5 to 9, wherein homogenisation
a. is performed at a pH of greater than about 7; or
b. is performed at a physiological pH; or
c. is performed under low shear conditions;
d. is performed in the presence of one or more stabilising additives, such as, for example, arginine; or
e. is performed at low temperature;
f. occurs at a temperature of from about 0° C. to about 5° C.;
g. is interspersed with rest phases in which no homogenising is performed, for example, interspersed with a chilling phase where the homogenate is placed on ice for a period; or
h. any combination of two or more of the above.

11. The method of any one of claims 5 to 10, wherein the substantial proportion of the purified crystallin proteins that retain their native structure is greater than about 60%.

12. A method of tissue closure in a subject in need thereof, the method comprising
optionally applying force to close the laceration, lesion, incision or wound;
contacting a laceration, lesion, incision, or wound or the site of said laceration, lesion, incision, or wound with a crystallin protein containing composition as defined in any one of the preceding claims, optionally wherein the crystallin protein containing composition is at least partially crosslinked,
optionally applying force to close the laceration, lesion, incision or wound,
initiating and/or maintaining crosslinking;
maintaining the closure of the laceration, lesion, incision or wound for a time sufficient for crosslinking to occur;
wherein crosslinking of the crystallin proteins forms an adhesive composition.

13. The method of claim 12, wherein the method of tissue closure is a method of closing a surgical incision.

14. The method of claim 12, wherein the method of tissue closure is a method of sutureless closure, for example, the sutureless closure is sutureless skin closure, sutureless wound closure, or sutureless operative incision closure.

15. The method of any one of claims 12 to 14, wherein the surgery is ophthalmic surgery.

16. The method of any one of claims 12 to 15, wherein maintenance of the closure of the laceration, lesion, incision or wound is
a. by the application of one or more medical aids, such as bandages, sutures, meshes or the like, or by (usually temporary) physical force, such as clamping or holding the laceration, lesion, incision or wound closed.
b. for a time sufficient for greater than about 60% crosslinking to occur; or
c. both a) and b) above.

17. The method of any one of claims 12 to 16, wherein the crosslinker present in the composition is a photocrosslinker, wherein initiation of crosslinking is by exposure to light.

18. A method of tissue closure in a subject in need thereof, wherein the subject is undergoing or who has undergone ophthalmic surgery, the method comprising
contacting a surgical incision or the site of said surgical incision with a crystallin protein containing composition as defined in any one of the preceding claims, optionally wherein the crystallin protein containing composition is at least partially crosslinked;
optionally applying force to close the incision;
initiating and/or maintaining crosslinking;
maintaining the closure of the surgical incision for a time sufficient for crosslinking to occur;
wherein crosslinking of the crystallin proteins forms an adhesive composition capable of maintaining closure of the surgical incision.

19. A method of treating an ocular injury or ocular incision in a subject in need thereof, the method comprising the steps:
contacting the ocular injury or incision with a composition as described herein, optionally wherein the crystallin protein containing composition is at least partially crosslinked; and
initiating and/or maintaining crosslinking;
wherein the crosslinking forms a bioadhesive polymer composition.

20. A method of delivering one or more active agents to a subject in need thereof, the method comprising
providing a crystallin protein comprising composition as defined in any one of the preceding claims, optionally wherein the crystallin protein containing composition is at least partially crosslinked, wherein the composition additionally comprises one or more active agents,
contacting the subject with the composition,
optionally initiating and/or maintaining crosslinking of the composition,
thereby delivering the active agent to the subject in need thereof.

21. The method of claim 20, wherein contacting the subject with the composition comprises administering the composition to a target site on or in the subject's body, including, for example, surgical administration.

22. A method of culturing one or more cells or tissues, the method comprising
providing one or more cells to be cultured;
contacting the one or more cells with a substrate comprising a composition as defined in any one of the preceding claims;
maintaining the one or more cells in contact with the substrate and optionally in contact with additional growth media for a period under conditions suitable for continued viability, growth, replication, and/or differentiation.

23. The method of claim 22, wherein the composition as described herein comprises γ-crystallin.

24. The method of claim 22 or 23, wherein the one or more cells comprise one or more replicatively-competent cells, or one or more stem cells.

25. The method of any one of claims 22 to 24, wherein the substrate is a thin film formed from a composition as defined in any one of the preceding claims, for example a thin film of sufficient mechanical strength and/or elasticity to enable the transfer of cells in contact therewith to another location.

26. The method of any one of claims 22 to 25, wherein the location is a second culture vessel.

27. The method of any one of claims 22 to 25, wherein the location is on or in a subject's body.

28. The method of claim 27, wherein one or more cells are one or more ophthalmic cells or one or more stem cells derived from the eye, and the location is a surgical site in or on the eye.

29. The method of claim 27 or 28, wherein one or more cells are one or more limbal stem cells, or one or more stromal stem cells.

30. The method of any one of claims 22 to 29, wherein the substrate is a gel formed from a composition as defined in any one of the preceding claims having at least one region of sufficient thickness to allow for the formation of a 3D cell culture.

31. The method of any one of claims 22 to 30, wherein the method of culturing one or more cells or tissues is a method of culturing one or more cells from the vertebrate eye, the method comprising
providing one or more vertebrate eye cells to be cultured;
contacting the one or more cells with a substrate comprising a composition as defined in any one of the preceding claims, wherein the substrate is optically transparent;
maintaining the one or more cells in contact with the substrate and optionally in contact with additional growth media for a period under conditions suitable for continued viability, growth, replication, and/or differentiation;
wherein the substrate is of sufficient mechanical durability to support transfer to the eye of a subject and/or handling associated with surgical application.

32. A method of treating an ocular disorder associated with deficiency of stem cells in a subject in need thereof, the method comprising contacting the eye with a therapeutic composition comprising:
i. a stem cell, optionally cultured according to a method of culturing as defined in any one of the preceding claims;
and optionally
ii. a biocompatible composition or biopolymer composition as defined in any one of the preceding claims.

33. A method of treating an ocular disorder in a subject in need thereof, the method comprising
providing a biocompatible composition as defined in any one of the preceding claims,
wherein the biocompatible composition comprises one or more active agents, and
administering the biocompatible composition to the subject to allow transfer of the one or more active agents to the subject.

34. A method of treating an ocular disorder in a subject in need thereof, the method comprising
providing a biocompatible composition as defined in any one of the preceding claims,
wherein the biocompatible composition comprises one or more stem cells, and
administering the biocompatible composition to the subject to allow transfer of one or more of the stem cells to the subject

35. Use of a composition as defined in any one of the preceding claims in the preparation of a medicament for use in therapy.

36. Use of a composition as defined in any one of the preceding claims in the preparation of a medicament or composition for in vitro use, including a therapeutic or research method that employs an in vitro step.

37. A composition as defined in any one of the preceding claims for use in therapy, including use in any one of the therapeutic methods described herein.

38. A composition as defined in any one of the preceding claims for use in an in vitro therapeutic or research method, or a therapeutic or research method that employs an in vitro step.

39. The composition, method or use of any one of the preceeding claims, wherein
a. the native secondary structure of the one or more crystallin proteins is maintained; or
b. the native tertiary structure of the one or more crystallin proteins is maintained; or
c. the native quaternary structure of the one or more crystallin proteins is maintained; or
d. the one or more crystallin proteins are substantially free of nanofibrils or other disrupted structural forms; or
e. at least some of the crystallin protein present in the composition is natively glycosylated; or
f. any combination of two or more of a) to e) above.

40. The composition, method or use of any one of the preceeding claims, wherein the composition comprises
a. from about 0.1% w/w to about 1.5% w/w crosslinker; or
b. from about 0.5% w/w to about 3% w/w crosslinker; or
c. from about 3% w/w to about 30% w/w crosslinker; or
d. from about 10 mg/mL to about 200 mg/mL crystallin protein; or
e. from about 10 mg/mL to about 120 mg/mL crystallin protein; or
f. from about 0.5% w/w to about 3% w/w plasticizer; or
g. from about 0.5% w/w to about 5% w/w co-initiator; or
h. any combination of two or more of a) to g) above.

41. The composition, method or use of any one of the preceeding claims, wherein the composition comprises
a. from about 100 mg/mL to about 120 mg/mL crystallin protein, from about 5 mM to about 10 mM glutaraldehyde, and from about 1.5% w/w to about 2.5% w/w glycerol; or
b. from about 100 mg/mL to about 120 mg/mL crystallin protein, from about 10% w/w to about 20% w/w PEGDA, and from about 0.2% w/w to about 1.0% w/w photoinitiator; or
c. from about 50 mg/mL to about 80 mg/mL crystallin protein, from about 10% w/w to about 20% w/w PEGDA, and from about 0.2% w/w to about 1.0% w/w photoinitiator; or
d. from about 50 mg/mL to about 80 mg/mL crystallin protein, from about 25% w/w to about 50% w/w PEGDA, from about 0.2% to about 1.0% w/w photoinitiator, and from 10% w/w to 20% w/w of co-initiator.

42. The composition, method or use of any one of the preceeding claims, wherein the composition when crosslinked
a. is optically transparent over the visible spectrum; or
b. has a refractive index equivalent to that of the eye of a subject to whom it is or has been administered; or
c. has a transmittance of light across the visible spectrum (400 nm to 700 nm) of greater than about 75%; or
d. any combination of two or more of a) to c) above.

43. The composition, method or use of any one of the preceeding claims, wherein when present the one or more active agents present in the composition is an ophthalmically acceptable antibiotic.

44. The composition, method or use of any one of the preceeding claims, wherein one or more of the crystallin proteins is from Hoki (Macruronus novaezelandiae), or is from Homo sapiens.
